(19) United States
(12) Patent Application Publication
(10) Pub. No.: US 2010/0129817 A1
(43) Pub. Date: May 27, 2010

(54) IDENTIFYING GERMLINE COMPETENT EMBRYONIC STEM CELLS

(75) Inventors: Yi Wei, New York, NY (US); Lynn MacDonald, White Plains, NY (US); Hsin Chieh Lin, Yorktown Heights, NY (US)

Correspondence Address: REGENERON PHARMACEUTICALS, INC, 777 OLD SAW MILL RIVER ROAD, TARRYTOWN, NY, 10591, US

(73) Assignee: Regeneron Pharmaceuticals Inc., Tarrytown, NY (US)

(21) Appl. No.: 12/597,694

(22) PCT Filed: May 2, 2008
(86) PCT No.: PCT/US08/62340

§ 371 Date: Oct. 26, 2009

Related U.S. Application Data
(60) Provisional application No. 60/927,568, filed on May 3, 2007.

(51) Int. Cl. C12Q 1/68; C40B 40/08; C40B 30/00

(52) U.S. Cl. 435/6; 506/17; 506/7

(57) ABSTRACT

Methods and compositions for selecting ES cells that are germline competent are provided, including gene expression arrays of from one to about 300 or more genes. Selecting ES cells that are competent for germline transmission by comparing the expression of one or more genes between an ES cell that is competent at germline transmission with an ES cell of interest is described. Selecting ES cells likely to be competent at germline transmission, based on their level of expression of gtl2, is also described.

### FIELD OF INVENTION

The field of invention is related to methods for identifying germline competent embryonic stem cells, including mouse embryonic stems cells, wherein the stem cells can include a genetic alteration that is a deletion, a mutation, and/or a transgene.

### BACKGROUND

An important step in making transgenic animals is assessing the likelihood that a particular ES cell transplanted into an embryo will contribute to the germline of the animal that will result from the embryo. Unless the ES cell contributes to the animal's germline, it is generally not possible to breed animals containing a desired genetic modification reflected in the ES cell's genome. The most common method for assessing the likelihood that an ES cell will transmit to the germline is karyotyping, or another cytological method. An abnormal karyotype generally is accepted as an indication that the ES cell would not be germline competent. But karyotyping, as well as other commonly employed cytological methods, is not very predictive of germline competence. For example, some ES cell clones that display normal karyotypes do not give rise to germline transmitting chimeras, or any chimeras at all. Further, karyotyping is a relatively laborious and expensive process that is not amenable to high throughput processing, but instead represents a significant bottleneck in producing transgenic animals. Accordingly, there is a need for better methods for identifying ES cells that are likely to be germline competent, including methods that are amenable to high throughput processing.

### SUMMARY

The invention is based at least in part on the ability to predict germline competency of ES cells by analyzing RNA expression in the ES cells. ES cells that are totipotent, and thus germline-competent, display an expression profile (i.e., identity of genes expressed and/or level of expression) that can be differentiated from non-totipotent ES cells.

In one aspect, a method for selecting an ES cell that is germline competent is provided, comprising providing an expression profile of a subset of ES cell genes from an ES cell, and comparing the expression profile of the subset of ES cell genes from the ES cell with an expression profile of a subset of ES cell genes from a germline competent ES cell.

In one embodiment, the profile is a profile of RNA. In a specific embodiment, the profile is a profile of mRNA. In one embodiment, the profile is a profile of expressed proteins. In a specific embodiment, the expressed proteins are proteins that are secreted. In a specific embodiment, the proteins are cell surface proteins.

In one embodiment, the expression profile is generated on an expression array.

In another embodiment, the expression profile is generated by an ELISA method or a protein expression assay.

In one embodiment, the expression profile is for a single gene. In one embodiment, an ES cell is germline competent if at least one preselected RNA is expressed at a level comparable to the level at which the at least one preselected RNA is expressed in a germline competent ES cell.

In another embodiment, the expression profile is a profile of expressed and secreted genes. In one embodiment, the expression profile is generated using an ELISA assay. In another embodiment, the expression profile is generated using an affinity assay using microspheres such as, for example, a Luminex assay.

In another embodiment, the subset of genes is about 292 genes. In another embodiment, the subset of genes is about 200 genes; in another embodiment, about 100 genes; in another embodiment, about 50 genes; in another embodiment, about 25 genes; in another embodiment, about 10 genes; in another embodiment, about five genes; in another embodiment, two genes; in another embodiment, one gene.

In one embodiment, the subset of genes is one or more genes listed in Table 8. In another embodiment, the subset of genes is one or more genes listed in Table 7. In another embodiment, the subset of genes is or more genes listed in Table 6. In another embodiment, the subset of genes is one or more genes listed in Table 5. In another embodiment, the subset of genes is one or more genes listed in Table 4. In another embodiment, the subset of genes is one or more genes listed in Table 3. In another embodiment, the subset of genes is one or more genes listed in Table 2. In another embodiment, the subset of genes is one or more genes listed in Table 1.

In another embodiment, the subset of genes is one or more genes listed in Table 8, in conjunction with one or more genes selected from the lists in Table 1 through Table 7. In another embodiment, the subset of genes consists of a single gene. In a specific embodiment, the single gene is gtl2.

In another aspect, a method for predicting germline competency for an ES cell is provided, comprising providing a subset of genes that are differentially expressed between germline competent ES cells and non-germline competent ES cells, ascertaining the level of expression of the subset of genes in an ES cell, and comparing the expression of the subset of genes in the ES cell to the expression of the subset of genes in a germline competent ES cell, wherein the ES cell is predicted to be germline competent if the expression profile is substantially similar to the expression profile of the ES cell that is germline competent.

In one embodiment, the expression profiles are substantially similar if at least 80% of the genes correlate with respect to up-regulation and down-regulation. In another embodiment, the expression profiles are substantially similar if at least 90% of the genes correlate with respect to up-regulation and down-regulation. In another embodiment, the expression profiles are substantially similar if at least 95% or more of the genes correlate with respect to up-regulation and down-regulation. In another embodiment, genes correlate where they do not differ by more than 100% when comparing expression; in another embodiment, they correlate where they do not differ by more than 50% when comparing expression level; in another embodiment, they correlate where they do not differ by more than 20% or less when comparing expression level.

In one embodiment, the prediction is at least 50% accurate. In another embodiment, the prediction is at least 60%, at least 70%, at least 80%, at least about 84%, at least 90%, or at least 95% accurate. In a specific embodiment, the prediction is at least 99% accurate. In one embodiment, accuracy of prediction is determined by whether the ES cell contributes to the germline in a chimeric animal into whose embryo the ES cell has been injected.

In another aspect, an expression array is provided, comprising a subset of genes of an ES cell that is differentially expressed in ES cells that are germline competent as compared with ES cells that are not germline competent.

In one embodiment, the subset of genes are selected from an ES cell genome based on the criterion that the subset of genes exhibit an up-regulation or down-regulation of at least two-fold between germline competent and non-germline competent ES cells. In one embodiment, the subset of genes exhibit a two- to three-fold difference between germline competent and non-germline competent ES cells. In another embodiment, the subset of genes exhibit a three- to four-fold difference between germline competent and non-germline competent ES cells. In another embodiment, the subset of genes exhibit at least a five-fold difference between germline competent and non-germline competent ES cells. In one embodiment, all genes of the subset exhibit about the same, cited fold difference in expression. In another embodiment, a majority of the genes of the subset exhibit the same, cited fold difference in expression.

In another aspect, use of an expression array to predict germline competency of a test ES cell is provided, comprising comparing an expression profile of a subset of genes whose expression differs between totipotent ES cells and non-totipotent ES cells, wherein an expression profile of a test ES cell that is substantially similar to an expression profile of a totipotent ES cell indicates that the test ES cell is germline competent.

In another aspect, a method is provided for selecting a cell from an ES cell population, wherein the selected cell has an increased likelihood of transmitting to the germline when injected into an embryo, comprising selecting an ES cell from the ES cell population whose expression profile for a preselected subset of genes is substantially similar to an expression profile of an ES cell that is germline competent.

Unless otherwise specified or apparent from the context, any feature of the invention can be used in conjunction with any other feature of the invention. Other objects and advantages will become apparent from a review of the ensuing detailed description.

### BRIEF DESCRIPTION OF THE FIGURES

FIG. 1 shows microarray analysis for two training sets of ES cells, which all had normal karyotypes; the first test set (two ES cell clones) were germline competent, whereas the second test set (two ES cell clones) were not germline competent as determined, in this example, by two microinjection attempts without chimeras.

FIG. 2 shows expression profile of test sets of ES cell clones (six clones) that had been targeted, and that had not been karyotyped.

FIG. 3 shows expression profile of a clone that was germline competent (“Germline Competent”), and the same clone differentiated (“Germline Competent differentiated”); “Non Germline Competent” clones are the “Non Germine Competent” clones of FIG. 2, presented for comparison.

FIG. 4 shows the percent of correct predictions of germline competency as a function of the number of genes analyzed in an expression array.

FIG. 5 shows the expression profile of a single gene (gtl2) in two germline competent ES cells (“Germline Competent 1”) and (“Germline Competent 2”); and its profile in two non-germline competent ES cell lines (“Germline Competent 1” and “Germline Competent 2”).

### DETAILED DESCRIPTION

The present invention is not limited to particular methods, and experimental conditions described, as such methods and conditions may vary. It is also to be understood that the terminology used herein is for the purpose of describing particular embodiments only, and is not intended to be limiting unless indicated, since the scope of the present invention will be limited only by the appended claims.

As used in this specification and the appended claims, the singular forms “a”, “an”, and “the” include plural references unless the context clearly dictates otherwise. Thus for example, references to “a method” include one or more methods, and/or steps of the type described herein and/or which will become apparent to those persons skilled in the art upon reading this disclosure.

Unless stated otherwise, all technical and scientific terms and phrases used herein have the same meaning as commonly understood by one of ordinary skill in the art to which the invention belongs. Although any methods and materials similar or equivalent to those described herein can be used in the practice or testing of the present invention, the preferred methods and materials are now described. All publications mentioned herein are incorporated herein by reference.

The invention is based at least in part on the realization that it is possible to assess the suitability of an ES cell for implantation in an embryo, such that the embryo develops into an animal wherein the ES cell contributes to the animal's germline.

The ability of an implanted ES cell to contribute to the development of an embryo and give rise to a chimeric animal that comprises the genetic material of the implanted ES cell, commonly referred to as germline competence, depends to a great extent on the totipotency of the ES cell.

As ES cells differentiate, they lose totipotency. Although ES cells that have differentiated and lost totipotency may retain some level of pluripotency, ES cells that are not totipotent are generally not useful in producing transgenic animals. ES cells lacking totipotency do not contribute to the germline of the animal and thus do not generally give rise to chimeras capable of transmitting the genetic material of the ES cell in the animal's germline—i.e., they are not generally germline competent. The differentiation state of an ES cell, or lack of differentiation, is reflected in the gene expression profile of the ES cell. Thus, based on an ES cell's expression profile, it may be possible to predict the potential of the ES cell to contribute to the germline of an embryo into which the ES cell is implanted.

The ability to predict with relative accuracy which ES cells in a population of ES cells, or between populations of ES cells, have the highest likelihood for germline competence is important. As germline competence drops, the number of ES cell clones that must be injected rises dramatically, as does the number of resulting chimeras that must be screened for germline competence. Injection and screening steps are both costly and time consuming. Further, proceeding in the face of a relatively unpredictable frequency of germline requires making more targeted clones (since more injections are necessary). Thus, increasing the likelihood that the ES clones injected are germline competent can result in large savings of resources and time.

However, beyond economic costs and adaptability to a high throughput set up, an assay that can reliably predict the GLT potential of an ES cell clone or line would also be of paramount utility in the following situations:

The ability to retain germline competence is a property that could be subject to or affected by the genetic background of the ES cell. Generally, inbred ES cells lines frequently display low germline transmission rates, with some B/6 lines displaying less than 40% germline transmission. Since the use of ES cells derived from inbred cell lines is becoming increasingly desirable, the ability to predict which targeted clones have a relatively high germline transmission rate would be of very high utility. For example, the methods and compositions provided herein would be of very high utility to large scale, high throughput knockout projects such as, for example, NIH's Knockout Mouse Project (KOMP).

In another aspect, improved methods for identifying germline competent ES cells would facilitate selecting ES cell clones that should be deposited into targeted ES cell repositories. Only those clones that have a relatively higher probability of germline competency will be stored, thus saving storage space and reducing the probability of spreading non germline competent clones throughout the scientific community.

In another aspect, improved methods for identifying germline competent ES cells would serve as a quality control step following thawing and expansion of clones for distribution purposes. Germline competency could be assessed prior to distribution, and clonal lines that have lost germline competency due to storage, handling, thawing, or expansion conditions can be eliminated prior to distribution.

In another aspect, improved methods for identifying germline competent ES cells would minimize the risk of using non-germline competent ES cell clones in projects that require multiple targeting steps, such as, for example, projects wherein large loci are sequentially modified, e.g., projects in which distal regions of the same chromosome must be modified, or projects that require repeated targeting in order to modify gene clusters or families of closely related genes.

In order to prepare a training set for comparing expression profiles and developing a method for predicting germline competency, two sets of ES cell clones were selected as training sets. The first set of ES cell clones (two clones in a set) had been sequentially targeted seven times using homologous recombination using standard homologous recombination methods. The second set (two clones in a set) had been sequentially targeted eight times. Both sets were karyotyped. All sets had normal karyotypes.

Each set of ES cells were grown cells were thawed, passed on feeder cells, and then grown on gelatin under standard culture conditions to prevent cell differentiation. The ES cells were F1H4 ES cells. Cells were grown in triplicate and expanded to six-well format. RNA was extracted from each well using Qiagen's RNAEasy kit. Microarray analysis was done on the RNA prepared from each clone.

For each data set, targeted ES cells were injected into blastocysts or 8-cell stage mouse embryos, and a total of 50 or more blastocysts were transformed into pseudo-pregnant female mice. If chimeras are born, they are tested for germline competency by determining whether they produced F1 heterozygotes. Germline transmission is defined as chimeras that are born that will produce an F1 heterozygote. Non-germline transmission is defined as the lack of production of chimera, or the production of a chimera that does not contribute to the germline.

Briefly, ES cell clones were thawed and plated the presence of Lif and grown on feeders (standard culture media for maintaining the undifferentiated phenotype). RNA was extracted using Qiagen's RNAEasy kit, purified RNA was labeled using Agilent's low-input RNA amplification kit, and hybridized to an Agilent mouse whole genome array (Cat. No. 4122A). Arrays were scanned using Agilent's scanner and data was extracted using Agilent's feature extraction software.

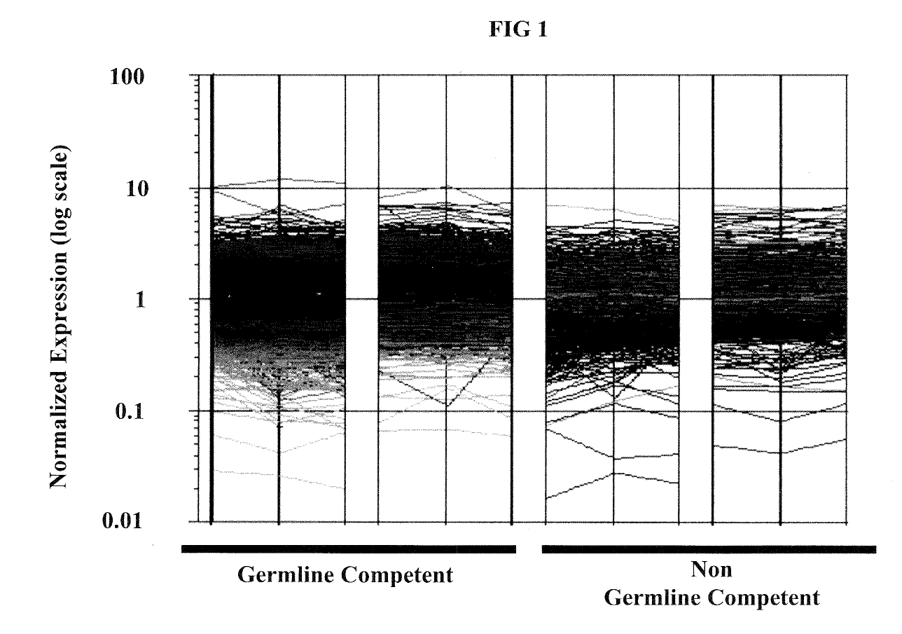

As shown in FIG. 1, microarray analysis each of the ES cell training sets revealed that the ES cells that went germline had similar expression profile s (see the two training sets, shown in triplicate, under “Germline Competent”). Genes above “1” represent up-regulated genes; genes below “1” represent down-regulated. As can be seen from the training set data of FIG. 1, germline competent ES cells have characteristic expression profile s of up-regulated and down-regulated genes. The second half of FIG. 1 (“Non Germline Competent”) shows two sets of non-germine transmitting ES cells. As can be seen by comparison with the expression profile for germline transmitting ES cells, many genes that are up-regulated in germline competent ES cells are down-regulated in non-germline competent ES cells, and vice versa. In short, FIG. 1 illustrates that a loss of totipotency correlates with an observable expression profile in ES cells. This expression profile can be predictive of loss of totipotency and, thus, of germline competency in ES cells.

In FIG. 1, “Germline Competent” is defined as germline transmission; “non germline competent” is defined in this experiment as failure of germline transmission after two microinjection attempts. The microarray used was an Agilent array (Cat. No. G4122A). The Agilent array comprises about 40,000 genes. The microarray profile shows expression of a subset of about 362 genes that represents a set of genes that are differentially expressed between germline competent and non-germline competent ES cells. The 362 genes (Table 1) were selected by using a t-test analysis on germline and non-germline competent array profiles, selecting genes that exhibit at least a 2-fold up-regulation or down-regulation between germline competent and non-germline competent ES cells. In each of the figures, each vertical line represents a single sample (results shown in triplicate; three samples per experiment).

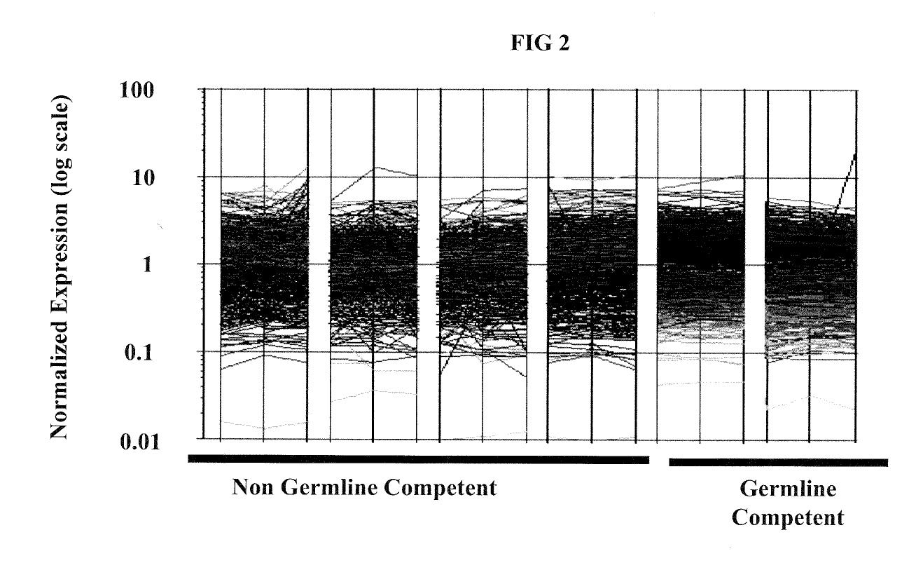

FIG. 2 shows expression profile of a test set of ES cell clones (six clones) that had been targeted, and that had not been karyotyped. Based on the expression profiling of the test set of FIG. 1, it is apparent from FIG. 2 that the final two clones (at right) would be expected to be germline competent based solely on the expression data shown, whereas the first four clones (at right) would be expected not to be germline competent.

Each clone of FIG. 2 was microinjected into a mouse blastocyst or a mouse embryo at the 8-cell stage. One of the “Germline Competent” clones at the right of FIG. 2 was determined to be germline competent (expression array profiling predicts that the other clone would be germline competent). The “Non Germline Competent” clones at the left of FIG. 2 were not germline competent.

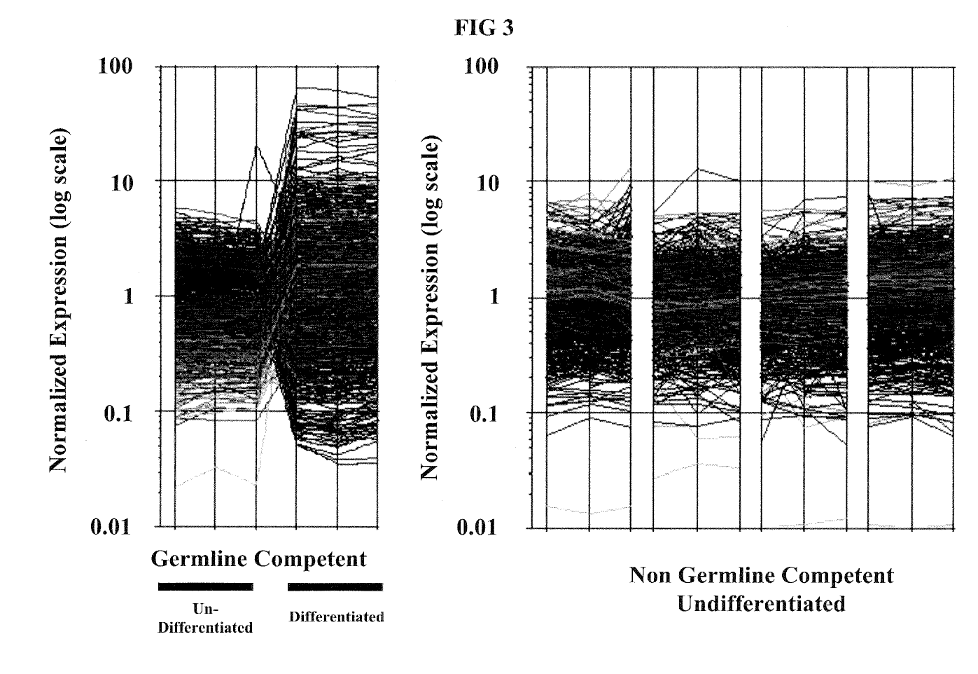

FIG. 3 shows expression profile of a clone that produced germline (“Germline Competent”), and the same clone differentiated (“Germline Competent differentiated”); “Non Germline Competent” clones are the “Non Germline Competent” clones of FIG. 2, presented for comparison. FIG. 3 illustrates that when a germline competent ES cell clone is differentiated (i.e., when it loses totipotency), its expression profile resembles that of a non-germline competent ES cell. For differentiation, ES cells were cultured in the absence of Lif, feeders, and gelatin, and grown in suspension for 14 days to develop EB bodies (embryonic bodies). EB bodies were trypsinized and plated out for a further seven days in the absence of Lif and in the absence of feeders and in the absence of gelatin, and RNA for expression profiling was prepared as described previously. No specific differentiation factors were administered.

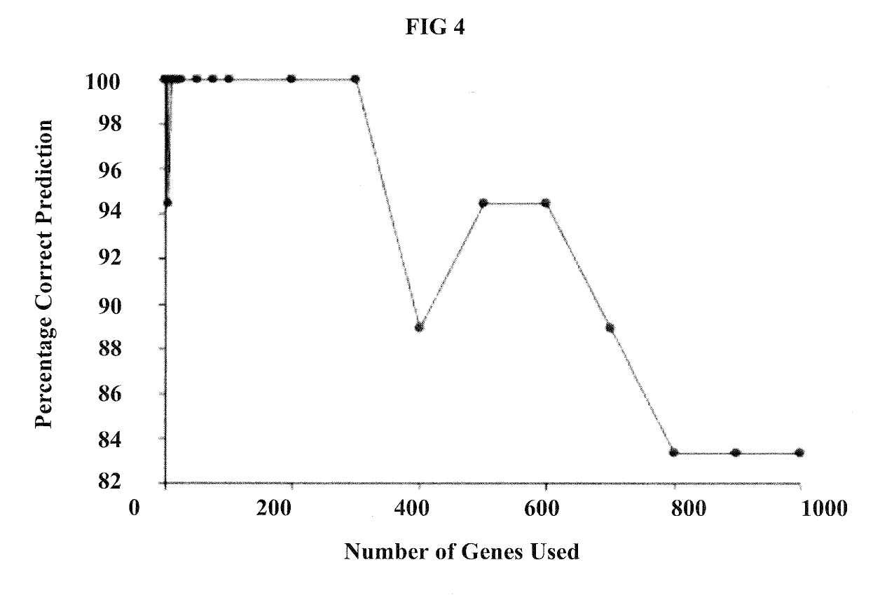

FIG. 4 shows the percent of correct predictions of germline competency of ES cells as a function of the number of genes analyzed in an expression array. The support vector machine class prediction method as implemented in GeneSpring (Agilent) was used. One hundred percent accuracy in predictive ability is achievable from about ten to about 292 genes. At least 93% accuracy is achievable from about five genes to about 25 genes. At ten genes or more, predictability is up to about 100%. The identities of the 362 genes used in the training set are shown in 6. The identities of the subsets of genes used in FIG. 4 are shown in Tables 1 through 8.

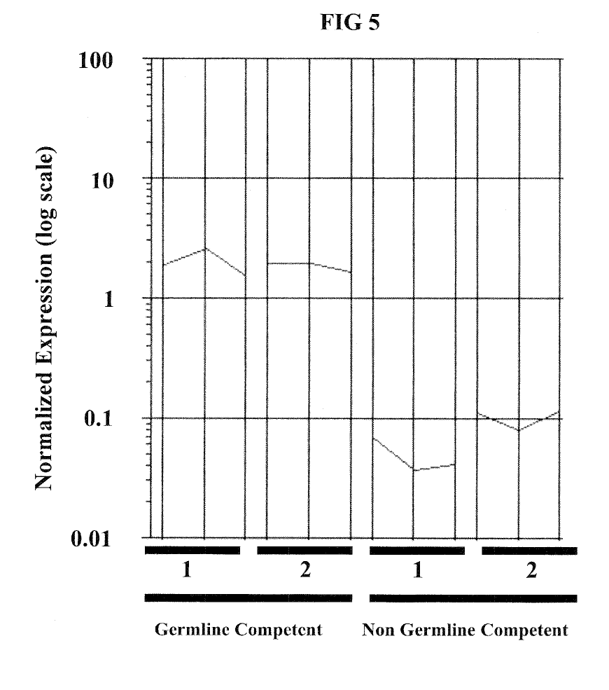

FIG. 5 shows the expression profile of a single gene (gtl2) in two germline competent ES cells (“Germline Competent 1”) and (“Germline Competent 2”); and its profile in two non-germline competent ES cell lines (“Non Germline Competent 1” and “Non Germline Competent 2”). As is apparent from the results shown in FIG. 5, it is possible to predict germline transmission competency using an expression profile for a single gene.

Table 1 shows a subset of 362 genes that were used as a predictor of germline competence. This subset of 362 genes was further reduced to 292 genes (Table 2), 200 genes (Table 3), 100 genes (Table 4), 50 genes (Table 5), 25 genes (Table 6), 10 genes (Table 7), and 5 genes (Table 8), and each subset displays an expression profile difference analogous to the difference shown in FIG. 1 for the set of 362 genes. Accordingly, relatively accurate predictions of germline competence can be made on subsets of genes as small as, for example, 5 genes. Further, accurate predictions can be made for subsets as small as 3 genes (FIG. 4), or one gene (FIG. 5).

TABLE 1A
Training Set Genes (Gemline Competent)

| | | | | Clone 1 | | | Clone 2 | | |
|---|---|---|---|---|---|---|---|---|---|
| Agilent Probe ID | Genbank | Entrez ID | Gene Symbol | rep. 1 | rep. 2 | rep. 3 | rep. 1 | rep. 2 | rep. 3 |
| A_51_P101975 | NM_010205 | 14179 | Fgf8 | 0.412 | 0.39 | 0.413 | 0.415 | 0.454 | 0.385 |
| A_51_P102081 | NM_007847 | 13222 | Defcr-rs2 | 0.061 | 0.0426 | 0.0644 | 0.0791 | 0.158 | 0.0864 |
| A_51_P102257 | AK053112; XM_619640 | 320477 | E030037J05Rik | 1.895 | 1.871 | 1.913 | 2.03 | 2.46 | 2.096 |
| A_51_P103397 | AK078521; | 54561 | Nap1l3 | 2.701 | 2.645 | 2.885 | 3.303 | 3.612 | 3.031 |
| A_51_P103406 | NM_011708 | 22371 | Vwf | 3.089 | 2.827 | 2.64 | 4.179 | 4.502 | 3.84 |
| A_51_P104162 | AK053379; | 328232 | | 2.537 | 3.096 | 3.391 | 3.126 | 2.998 | 3.127 |
| A_51_P105068 | NM_027990 | 71897 | 2310010M24Rik | 0.459 | 0.371 | 0.462 | 0.48 | 0.484 | 0.498 |
| A_51_P106918 | NM_009051 | 19715 | Rex2 | 2.006 | 2.032 | 2.425 | 6.886 | 2.95 | 2.717 |
| A_51_P109649 | AK018142; | 17263 | Gtl2 | 1.183 | 1.072 | 1.216 | 1.06 | 1.024 | 0.976 |
| A_51_P113162 | AK086843; | | | 1.844 | 2.453 | 2.29 | 2.743 | 2.858 | 1.748 |
| A_51_P114606 | NM_016965 | 50884 | Nckap1 | 0.446 | 0.473 | 0.337 | 0.707 | 0.582 | 0.773 |
| A_51_P115005 | NM_010104 | 13614 | Edn1 | 0.183 | 0.137 | 0.0678 | 0.336 | 0.288 | 0.302 |
| A_51_P121635 | NM_030700 | 80884 | Maged2 | 0.378 | 0.371 | 0.409 | 0.492 | 0.475 | 0.481 |
| A_51_P124315 | NM_177649 | 227712 | BC034076 | 1.796 | 1.742 | 1.797 | 2.396 | 2.68 | 2.14 |
| A_51_P125679 | AK010725; XM_134948 | 73673 | 2410076I21Rik | 2.867 | 2.332 | 2.464 | 3.518 | 3.661 | 3.727 |
| A_51_P127560 | AK051724; | 76577 | | 1.567 | 2.006 | 1.603 | 1.713 | 1.753 | 1.298 |
| A_51_P130095 | NM_010187 | 14130 | Fcgr2b | 1.115 | 0.945 | 1.055 | 1.587 | 1.618 | 1.515 |
| A_51_P140751 | NM_011397 | 20522 | Slc23a1 | 1.819 | 1.664 | 1.85 | 2.855 | 2.607 | 2.546 |
| A_51_P141660 | AK053665; XM_358382 | 67776; 235380 | Loh11cr2a | 2.996 | 3.339 | 3.08 | 2.433 | 2.601 | 2.365 |
| A_51_P144417 | NM_181750 | 226412 | R3hdm | 2.79 | 2.726 | 2.677 | 2.283 | 2.582 | 2.164 |
| A_51_P144712 | NM_178930 | 107338 | Gbf1 | 1.805 | 1.74 | 1.841 | 1.291 | 1.456 | 1.202 |
| A_51_P147651 | AK041797; XM_146705 | 245902 | | 2.797 | 2.965 | 2.694 | 2.18 | 2.325 | 1.652 |
| A_51_P149469 | NM_009608 | 11464 | Actc1 | 0.324 | 0.387 | 0.384 | 0.496 | 0.551 | 0.471 |
| A_51_P153079 | NM_133923 | 101100 | 4833441J24Rik | 1.312 | 1.174 | 1.271 | 1.222 | 1.343 | 1.299 |
| A_51_P157986 | NM_029930 | 77574 | 3321401G04Rik | 0.426 | 0.339 | 0.345 | 0.368 | 0.318 | 0.37 |
| A_51_P158037 | NM_013505 | 13506 | Dsc2 | 0.271 | 0.249 | 0.258 | 0.437 | 0.415 | 0.453 |
| A_51_P160160 | AK017624; | 70548 | 5730437C12Rik | 2.323 | 2.175 | 2.466 | 2.08 | 1.666 | 3.278 |
| A_51_P161115 | AK050756; | | | 2.088 | 3.879 | 2.31 | 2.955 | 2.678 | 1.887 |
| A_51_P162474 | AF285577; | 107815 | Scml2 | 0.539 | 0.413 | 0.405 | 0.643 | 0.645 | 0.71 |
| A_51_P162481 | NM_133194 | 107815 | Scml2 | 0.432 | 0.357 | 0.385 | 0.594 | 0.55 | 0.618 |
| A_51_P164219 | NM_011909 | 24110 | Usp18 | 0.435 | 0.307 | 0.303 | 0.435 | 0.448 | 0.457 |
| A_51_P164504 | NM_007469 | 11812 | Apoc1 | 3.245 | 2.908 | 3.052 | 2.863 | 3.059 | 2.591 |
| A_51_P165082 | AB041601; XM_489523 | 224019 | D16Bwg1494e | 3.114 | 3.318 | 3.453 | 3.352 | 3.568 | 3.01 |
| A_51_P165244 | NM_018734 | 55932 | Gbp4 | 0.585 | 0.512 | 0.474 | 1.008 | 0.981 | 1.006 |
| A_51_P168646 | AK039580; | | | 0.442 | 0.434 | 0.509 | 0.537 | 0.548 | 0.598 |
| A_51_P171728 | NM_011926 | 26365 | Ceacam1 | 2.703 | 1.992 | 2.013 | 3.11 | 3.457 | 2.977 |
| A_51_P174943 | NM_008485 | 16782 | Lamc2 | 0.234 | 0.255 | 0.286 | 0.433 | 0.411 | 0.451 |
| A_51_P176474 | AK010466; | 76484 | 2410012C07Rik | 2.797 | 2.691 | 2.733 | 3.87 | 4.072 | 3.726 |
| A_51_P180413 | NM_176930 | 319504 | C130076O07Rik | 0.359 | 0.393 | 0.415 | 0.471 | 0.514 | 0.494 |
| A_51_P187645 | BC004786; XM_354700 | 16061 | Igh-VJ558 | 0.714 | 0.737 | 0.706 | 0.927 | 0.803 | 0.637 |
| A_51_P189631 | NM_172427 | 76455 | 2310067E19Rik | 1.139 | 0.941 | 0.985 | 1.62 | 1.653 | 1.604 |
| A_51_P191990 | AK087548; | 26563 | Ror1 | 1.628 | 1.623 | 1.626 | 1.509 | 1.588 | 1.479 |
| A_51_P193019 | NM_011157 | 19073 | Prg1 | 1.283 | 1.107 | 1.495 | 0.759 | 3.853 | 1.325 |
| A_51_P199888 | NM_026082 | 67299 | Dock7 | 0.407 | 0.403 | 0.245 | 0.558 | 0.428 | 0.546 |
| A_51_P200223 | XM_619497 | | | 2.332 | 2.259 | 2.383 | 1.575 | 1.797 | 1.553 |
| A_51_P202911 | NM_144882 | 67198 | 2810022L02Rik | 0.297 | 0.283 | 0.328 | 0.346 | 0.333 | 0.365 |
| A_51_P203955 | NM_010260 | 14469 | Gbp2 | 0.252 | 0.267 | 0.292 | 0.582 | 0.578 | 0.664 |
| A_51_P207934 | NM_199016 | 224794 | Enpp4 | 2.477 | 2.57 | 2.229 | 2.606 | 2.707 | 2.427 |
| A_51_P219166 | NM_027756 | 71306 | Mfap3l | 0.459 | 0.46 | 0.497 | 0.549 | 0.502 | 0.507 |
| A_51_P221291 | NM_009229 | 20650 | Sntb2 | 1.876 | 1.793 | 1.783 | 1.622 | 1.693 | 1.524 |
| A_51_P222773 | NM_010446 | 15376 | Foxa2 | 0.423 | 0.314 | 0.4 | 0.447 | 0.809 | 0.489 |
| A_51_P223132 | NM_031183; XM_486227 | 83395; 70549 | Sp6 | 2.329 | 2.088 | 2.196 | 1.457 | 1.639 | 1.36 |
| A_51_P223656 | NM_008484 | 16780 | Lamb3 | 0.447 | 0.289 | 0.361 | 0.538 | 0.544 | 0.402 |
| A_51_P228472 | NM_008343 | 16009 | Igfbp3 | 0.288 | 0.22 | 0.276 | 0.283 | 0.322 | 0.364 |
| A_51_P229613 | NM_008792 | 18549 | Pcsk2 | 0.512 | 0.483 | 0.515 | 0.58 | 0.58 | 0.504 |
| A_51_P231341 | AK087764; | 108160 | D0HXS9928E | 2.885 | 3.795 | 2.39 | 3.305 | 3.578 | 2.451 |
| A_51_P236267 | NM_009183 | 20452 | St8sia4 | 0.265 | 0.182 | 0.148 | 0.233 | 0.291 | 0.32 |
| A_51_P237040 | NM_008711 | 18121 | Nog | 0.266 | 0.299 | 0.281 | 0.396 | 0.368 | 0.407 |
| A_51_P240253 | NM_019662 | 56437 | Rrad | 0.316 | 0.322 | 0.314 | 0.392 | 0.337 | 0.342 |
| A_51_P240811 | NM_009290 | 20890 | Wnt8a | 0.709 | 0.589 | 0.527 | 1.532 | 0.979 | 1.578 |
| A_51_P242399 | NM_031170 | 16691 | Krt2-8 | 0.193 | 0.233 | 0.208 | 0.403 | 0.4 | 0.39 |
| A_51_P252565 | NM_009660 | 11687 | Alox15 | 0.236 | 0.24 | 0.156 | 0.217 | 0.231 | 0.212 |
| A_51_P253053 | XM_618867 | | | 2.544 | 3.669 | 3.053 | 2.284 | 2.658 | 3.007 |
| A_51_P262171 | NM_008326 | 15944 | Ifi1 | 0.29 | 0.264 | 0.261 | 0.474 | 0.475 | 0.507 |
| A_51_P266403 | AK009163; | 76730 | 2310005C01Rik | 3.079 | 3.053 | 3.092 | 2.733 | 2.967 | 2.734 |
| A_51_P266644 | BC055753; XM_125538 | 140742 | | 0.424 | 0.369 | 0.474 | 0.821 | 0.739 | 0.949 |
| A_51_P268167 | BC037232; XM_621365 | 229363; 218850 | Gmps | 2.665 | 2.898 | 2.35 | 2.187 | 2.366 | 2.198 |
| A_51_P270725 | AK089493; | 102093 | Phkb | 2.083 | 3.565 | 2.709 | 2.953 | 2.589 | 2.102 |
| A_51_P272363 | NM_134160 | 171166 | Mcoln3 | 0.559 | 0.82 | 0.792 | 0.686 | 0.762 | 0.915 |
| A_51_P275435 | NM_172411 | 71874 | 2310007B03Rik | 0.368 | 0.492 | 0.206 | 0.875 | 0.83 | 0.857 |
| A_51_P276305 | NM_009292 | 20899 | Stra8 | 3.137 | 3.069 | 3.174 | 2.824 | 2.972 | 2.777 |
| A_51_P282227 | NM_020577 | 57344 | As3mt | 2.651 | 2.607 | 2.876 | 3.224 | 3.327 | 3.147 |
| A_51_P283300 | NM_023116 | 12296 | Cacnb2 | 0.373 | 0.27 | 0.335 | 0.375 | 0.321 | 0.398 |
| A_51_P285725 | NM_009309 | 20997 | T | 0.141 | 0.102 | 0.113 | 0.134 | 0.13 | 0.141 |
| A_51_P289341 | AK030946; XM_141372 | 241639 | 5830467J12Rik | 0.436 | 0.286 | 0.249 | 0.623 | 0.601 | 0.689 |
| A_51_P291227 | NM_001005508 | 226652 | 6030405P05Rik | 1.876 | 1.826 | 1.86 | 2.054 | 2.185 | 2.004 |
| A_51_P294505 | NM_025658 | 66607 | Ms4a4d | 0.63 | 0.414 | 0.365 | 0.532 | 0.521 | 0.629 |
| A_51_P294566 | AK020687; | 78083 | 9930116N10Rik | 2.623 | 2.571 | 2.706 | 3.706 | 4.077 | 3.155 |
| A_51_P297131 | NM_009502 | 22330 | Vcl | 0.402 | 0.372 | 0.378 | 0.411 | 0.377 | 0.401 |
| A_51_P298266 | NM_025681 | 66643 | Lix1 | 0.507 | 0.414 | 0.416 | 0.554 | 0.584 | 0.54 |
| A_51_P302377 | NM_008009 | 14181 | Fgfbp1 | 0.258 | 0.246 | 0.273 | 0.413 | 0.381 | 0.451 |
| A_51_P305019 | AK020601; | 78423 | 9530049O05Rik | 1.297 | 1.416 | 1.484 | 1.43 | 1.551 | 1.387 |
| A_51_P311038 | BC009666; XM_134720 | 57267 | Apba3 | 0.334 | 0.341 | 0.339 | 0.446 | 0.472 | 0.482 |
| A_51_P316733 | AK173008; | 330914 | Grit | 1.501 | 1.439 | 1.367 | 1.586 | 1.703 | 1.516 |
| A_51_P318577 | AK010729; | 73671 | 2410078J06Rik | 3.283 | 4.227 | 3.815 | 6.599 | 6.454 | 5.366 |
| A_51_P321579 | XM_131865; XM_196334 | 433809 | LOC433809 | 2.839 | 2.589 | 2.323 | 1.805 | 1.942 | 1.521 |
| A_51_P327011 | NM_016744 | 18573 | Pde1a | 0.252 | 0.126 | 0.236 | 0.377 | 0.349 | 0.295 |
| A_51_P330380 | XM_130138; XM_145466 | 243881 | 4921517N04Rik | 0.448 | 0.326 | 0.174 | 0.467 | 0.44 | 0.467 |
| A_51_P333253 | NM_178440 | 246177 | Myo1g | 1.419 | 1.413 | 1.621 | 1.544 | 1.504 | 1.394 |
| A_51_P334093 | NM_026228 | 67547 | Slc39a8 | 0.208 | 0.0771 | 0.105 | 0.19 | 0.198 | 0.231 |
| A_51_P335460 | NM_009132 | 20259 | Scin | 0.718 | 0.631 | 0.719 | 0.678 | 0.674 | 0.666 |
| A_51_P337125 | NM_010566 | 16331 | Inpp5d | 3.162 | 2.936 | 3.033 | 4.399 | 4.495 | 4.161 |
| A_51_P341137 | AK035176; | 224829 | Trerf1 | 1.935 | 1.833 | 2.021 | 2.024 | 2.334 | 1.984 |
| A_51_P343129 | AK012079; | 70472 | Atad2 | 3.752 | 4.107 | 4.116 | 2.307 | 2.496 | 1.87 |
| A_51_P345422 | NM_010747 | 17096 | Lyn | 0.244 | 0.247 | 0.256 | 0.373 | 0.392 | 0.424 |
| A_51_P352572 | NM_019701 | 56365 | Clcnkb | 5.193 | 4.267 | 4.344 | 4.514 | 5.226 | 4.635 |
| A_51_P353221 | NM_011582 | 21828 | Thbs4 | 0.342 | 0.418 | 0.408 | 0.589 | 0.504 | 0.543 |
| A_51_P354265 | NM_009256 | 20723 | Serpinb9 | 0.132 | 0.108 | 0.0848 | 0.29 | 0.281 | 0.346 |
| A_51_P354307 | NM_172469 | 209195 | Clic6 | 0.43 | 0.319 | 0.329 | 0.398 | 0.398 | 0.387 |
| A_51_P356003 | NM_001007580 | 333564 | Gm784 | 0.494 | 0.445 | 0.513 | 0.509 | 0.483 | 0.534 |
| A_51_P356883 | NM_008728 | 18162 | Npr3 | 0.397 | 0.367 | 0.304 | 0.418 | 0.383 | 0.399 |
| A_51_P358344 | NM_007732 | 12821 | Col17a1 | 3.116 | 2.917 | 3.011 | 3.334 | 3.243 | 2.913 |
| A_51_P358714 | AK031433; | | | 2.043 | 2.448 | 1.68 | 2.647 | 2.627 | 1.899 |
| A_51_P378051 | NM_009675 | 11754 | Aoc3 | 2.988 | 3.162 | 3.341 | 3.251 | 3.583 | 3.381 |
| A_51_P378371 | | | | 0.454 | 0.542 | 0.39 | 0.468 | 0.484 | 0.468 |
| A_51_P381069 | XM_132045 | 231252 | Chrna9 | 1.424 | 2.361 | 3.023 | 3.047 | 2.764 | 2.469 |
| A_51_P387528 | AK019664; | 78801 | Ak7 | 2.693 | 2.814 | 2.57 | 3.786 | 3.834 | 3.71 |
| A_51_P393518 | BC016539; | 240793 | | 0.393 | 0.238 | 0.292 | 0.344 | 0.296 | 0.462 |
| A_51_P397003 | XM_133990 | 68904 | 1110065L07Rik | 0.45 | 0.438 | 0.438 | 0.542 | 0.479 | 0.613 |
| A_51_P398766 | NM_010259 | 14468 | Gbp1 | 0.374 | 0.372 | 0.361 | 0.894 | 0.876 | 0.949 |
| A_51_P404193 | NM_022435 | 64406 | Sp5 | 0.194 | 0.182 | 0.19 | 0.214 | 0.202 | 0.207 |
| A_51_P405375 | NM_175358 | 108672 | Zdhhc15 | 0.458 | 0.438 | 0.301 | 0.668 | 0.553 | 0.495 |
| A_51_P407323 | NM_007976 | 14067 | F5 | 0.17 | 0.072 | NO DATA | 0.352 | 0.305 | 0.242 |
| A_51_P410813 | AK079732; | | | 2.732 | 2.716 | 2.559 | 2.898 | 2.605 | 2.3 |
| A_51_P412817 | NM_177617 | 216851 | D330014H01Rik | 3.151 | 3.039 | 3.326 | 2.136 | 2.194 | 1.865 |
| A_51_P418029 | NM_175541 | 245631 | Mum1l1 | 0.319 | 0.265 | 0.278 | 0.419 | 0.397 | 0.534 |
| A_51_P418375 | NM_023844 | 67374 | Jam2 | 1.691 | 1.467 | 1.625 | 2.142 | 2.132 | 2.288 |
| A_51_P418884 | NM_001013789 | 432823 | | 1.678 | 2.207 | 2.11 | 2.759 | 2.736 | 2.272 |
| A_51_P423859 | NM_007601 | 12335 | Capn3 | 2.32 | 2.027 | 1.637 | 2.955 | 2.666 | 3.034 |
| A_51_P437050 | BC072664; | 233328 | Lrrk1 | 0.339 | 0.28 | 0.261 | 0.525 | 0.503 | 0.498 |
| A_51_P442894 | AK048310; | 621603 | C130048D07Rik | 2.632 | 2.364 | 2.281 | 3.657 | 3.714 | 3.102 |
| A_51_P447189 | AK041326; XM_358090 | 385154 | | 2.336 | 2.35 | 2.641 | 3.32 | 2.976 | 3.108 |
| A_51_P455647 | NM_009801 | 12349 | Car2 | 0.335 | 0.288 | 0.301 | 0.568 | 0.516 | 0.638 |
| A_51_P455997 | Y13832; | 17263 | Gtl2 | 1.505 | 1.455 | 1.44 | 0.998 | 1.082 | 0.998 |
| A_51_P457187 | NM_028075 | 72049 | Tnfrsf13c | 2.82 | 3.275 | 3.026 | 2.654 | 3.432 | 2.546 |
| A_51_P463789 | BC051947; | 58212 | | 3.253 | 2.961 | 3.321 | 2.093 | 2.306 | 1.934 |
| A_51_P463791 | BC051947; | 58212 | | 3.074 | 2.866 | 2.865 | 2.058 | 2.064 | 1.757 |
| A_51_P463846 | NM_145545 | 229900 | 9830147J24Rik | 0.694 | 0.519 | 0.59 | 0.86 | 0.816 | 0.817 |
| A_51_P467505 | NM_013689 | 21682 | Tec | 0.28 | 0.242 | 0.227 | 0.502 | 0.464 | 0.499 |
| A_51_P473953 | XM_358600; XM_355454 | 381498 | 4930592C13Rik | 0.42 | 0.306 | 0.298 | 0.365 | 0.365 | 0.38 |
| A_51_P478930 | AK080016; | | | 2.436 | 2.701 | 2.337 | 2.779 | 2.919 | 2.466 |
| A_51_P480311 | NM_010168 | 14061 | F2 | 1.991 | 1.809 | 1.969 | 2.465 | 2.4 | 1.874 |
| A_51_P481777 | NM_018756 | 54382 | Tcstv1 | 1.532 | 1.822 | 1.792 | 2.619 | 3.155 | 2.956 |
| A_51_P481958 | NM_016674 | 12737 | Cldn1 | 0.867 | 0.572 | 0.569 | 0.535 | 0.328 | 0.499 |
| A_51_P485985 | NM_134163 | 171170 | Mbnl3 | 0.223 | 0.181 | 0.161 | 0.625 | 0.543 | 0.697 |
| A_51_P487004 | NM_027732 | 71241 | Dmrtc2 | 10.3 | 11.69 | 10.98 | 5.024 | 6.192 | 5.685 |
| A_51_P493682 | XM_134870; XM_140758 | 72672 | Rasl12 | 2.563 | 2.641 | 2.403 | 3.095 | 3.051 | 2.581 |
| A_51_P497100 | NM_010706 | 16855 | Lgals4 | 2.115 | 2.049 | 2.368 | 1.716 | 1.861 | 1.527 |
| A_51_P499854 | NM_010284 | 14600 | Ghr | 0.407 | 0.375 | 0.412 | 0.413 | 0.429 | 0.489 |
| A_51_P507880 | NM_028980 | 74521 | 8430415E04Rik | 0.223 | 0.221 | 0.235 | 0.35 | 0.326 | 0.332 |
| A_51_P512085 | NM_130449 | 140792 | Colec12 | 0.335 | 0.364 | 0.296 | 0.382 | 0.368 | 0.385 |
| A_51_P512172 | AK010953; XM_125673 | 52463 | Cxxc6 | 2.864 | 2.384 | 2.216 | 2.184 | 2.11 | 2.068 |
| A_51_P514177 | NM_028838 | 74249 | Lrrc2 | 3.542 | 3.2 | 3.896 | 3.884 | 4.114 | 3.603 |
| A_51_P520175 | AK076272; | 12223 | Btc | 0.505 | 0.139 | 0.208 | 0.462 | 0.57 | 0.379 |
| A_51_P521084 | NM_025626 | 66540 | 3110001A13Rik | 0.408 | 0.406 | 0.391 | 0.474 | 0.456 | 0.494 |
| A_52_P100042 | AK045143; | 320938 | Tnpo3 | 1.761 | 1.695 | 1.74 | 1.697 | 1.665 | 1.526 |
| A_52_P1011737 | AK039096; | 106258 | AI790442 | 1.846 | 1.977 | 1.978 | 2.087 | 2.67 | 2.065 |
| A_52_P1013441 | | | | 3.248 | 3.164 | 3.191 | 4.558 | 5.61 | 4.683 |
| A_52_P103929 | NM_009306 | 20979 | Syt1 | 0.382 | 0.356 | 0.39 | 0.419 | 0.498 | 0.423 |
| A_52_P1044262 | AK051949; | | | 1.478 | 1.257 | 1.19 | 1.611 | 1.769 | 1.41 |
| A_52_P1044696 | AK087281; | | | 1.535 | 1.733 | 1.77 | 1.994 | 2.551 | 1.772 |
| A_52_P1075773 | AK036921; | 399602 | 9930024M15Rik | 1.958 | 2.689 | 1.619 | 2.065 | 2.285 | 1.59 |
| A_52_P1084149 | AK051413; | 553138 | 9930021J03Rik | 1.669 | 1.913 | 1.945 | 1.806 | 2.202 | 1.595 |
| A_52_P1108139 | AK053079; | | | 1.801 | 2.214 | 1.538 | 1.924 | 2.168 | 1.724 |
| A_52_P1124598 | AK081090; | | | 1.689 | 1.334 | 1.065 | 1.621 | 1.94 | 1.111 |
| A_52_P113190 | NM_053214 | 17916 | Myo1f | 1.428 | 1.513 | 1.396 | 1.988 | 1.959 | 2.083 |
| A_52_P1172548 | AK084146; | 13204 | Dhx15 | 2.924 | 3.082 | 2.339 | 3.164 | 3.492 | 2.732 |
| A_52_P1180315 | AK049088; | 74022 | 3930401K13Rik | 1.789 | 1.737 | 1.505 | 1.313 | 1.364 | 1.207 |
| A_52_P1188270 | AK042362; | | | 3.076 | 1.828 | 1.917 | 2.932 | 3.046 | 2.87 |
| A_52_P1196751 | AK086488; | | | 2.217 | 2.193 | 2.198 | 2.38 | 2.677 | 2.156 |
| A_52_P126158 | NM_008326 | 15944 | Ifi1 | 0.237 | 0.208 | 0.22 | 0.415 | 0.412 | 0.425 |
| A_52_P129428 | BC049774; | 57265 | Fzd2 | 0.482 | 0.364 | 0.221 | 0.539 | 0.518 | 0.547 |
| A_52_P132608 | NM_172685 | 229731 | Slc25a24 | 0.358 | 0.291 | 0.25 | 0.504 | 0.401 | 0.432 |
| A_52_P135873 | XM_132015; XM_488584 | 231128 | BC037112 | 1.243 | 1.332 | 1.392 | 1.507 | 1.53 | 1.464 |
| A_52_P13815 | NM_010686 | 16792 | Laptm5 | 0.991 | 1.234 | 0.785 | 1.685 | 1.566 | 1.469 |
| A_52_P147870 | NM_029963 | 77721 | Mrps5 | 2.14 | 2.028 | 2.027 | 1.789 | 1.827 | 1.856 |
| A_52_P148222 | BC055037; | 224139 | 4930428L02Rik | 0.273 | 0.151 | 0.238 | 0.426 | 0.424 | 0.55 |
| A_52_P152983 | AK011739; | 26886 | Cenph | 2.134 | 3.107 | 2.171 | 2.114 | 2.408 | 2.327 |
| A_52_P153685 | AK129323; | 70422 | 2810417D08Rik | 0.452 | 0.397 | 0.407 | 0.587 | 0.526 | 0.735 |
| A_52_P159095 | AK051960; | 17685 | | 2.867 | 2.413 | 1.869 | 3.388 | 3.17 | 2.951 |
| A_52_P163148 | AB010370; | 544945 | | 2.883 | 2.86 | 2.64 | 3.58 | 3.156 | 3.085 |
| A_52_P164524 | NM_013628 | 18548 | Pcsk1 | 0.728 | 0.727 | 0.407 | 0.826 | 0.801 | 0.71 |
| A_52_P165497 | NM_011385 | 20481 | Ski | 2.312 | 2.37 | 2.474 | 1.296 | 1.485 | 1.406 |
| A_52_P165610 | NM_153534 | 210044 | Adcy2 | 0.452 | 0.354 | 0.394 | 0.474 | 0.421 | 0.374 |
| A_52_P16973 | AK081934; | 320391 | D930017J03Rik | 2.586 | 2.277 | 2.281 | 2.137 | 2.272 | 1.872 |
| A_52_P175685 | NM_008390 | 16362 | Irf1 | 0.401 | 0.37 | 0.3 | 0.663 | 0.637 | 0.749 |
| A_52_P176333 | NM_001005787 | 12695 | Cipp | 0.372 | 0.357 | 0.365 | 0.452 | 0.392 | 0.42 |
| A_52_P179250 | AK129127; | 230249 | AI314180 | 5.166 | 6.292 | 4.565 | 8.052 | 10.48 | 5.993 |
| A_52_P183368 | NM_011535 | 21386 | Tbx3 | 1.964 | 1.81 | 1.931 | 2.023 | 2.272 | 2.026 |
| A_52_P18887 | NM_011468 | 20755 | Sprr2a | 0.21 | 0.171 | 0.214 | 0.282 | 0.277 | 0.25 |
| A_52_P196568 | NM_144513 | 17263 | Gtl2 | 1.892 | 2.538 | 1.516 | 1.938 | 1.959 | 1.652 |
| A_52_P197826 | NM_010397 | 15039 | H2-T22 | 0.541 | 0.505 | 0.41 | 0.653 | 0.634 | 0.622 |
| A_52_P199633 | NM_199146 | 209387 | LOC209387 | 0.492 | 0.426 | 0.236 | 0.608 | 0.439 | 0.597 |
| A_52_P199922 | | | | 3.785 | 1.908 | 2.417 | 2.334 | 2.502 | 2.545 |
| A_52_P200458 | NM_010242 | 14345 | Fut4 | 0.429 | 0.343 | 0.355 | 0.453 | 0.391 | 0.512 |
| A_52_P206573 | NM_027427 | 70439 | Taf15 | 2.14 | 3.003 | 2.282 | 1.904 | 2.123 | 1.956 |
| A_52_P211956 | NM_025658 | 66607 | Ms4a4d | 0.61 | 0.455 | 0.457 | 0.534 | 0.519 | 0.685 |
| A_52_P214042 | NM_028125 | 72147 | Btbd4 | 0.512 | 0.42 | 0.228 | 0.756 | 0.669 | 0.611 |
| A_52_P21595 | NM_028238 | 72433 | Rab38 | 0.234 | 0.188 | 0.168 | 0.439 | 0.467 | 0.472 |
| A_52_P21628 | NM_019978 | 13175 | Dcamkl1 | 0.543 | 0.459 | 0.338 | 0.471 | 0.5 | 0.397 |
| A_52_P223083 | BC038323; | 72096 | 2010208K18Rik | 4.486 | 6.15 | 3.745 | 3.784 | 3.918 | 3.378 |
| A_52_P224125 | XM_622260 | | | 1.795 | 1.653 | 1.727 | 1.625 | 1.654 | 1.663 |
| A_52_P233673 | NM_020594 | 57432 | Zc3hdc8 | 3.001 | 3.71 | 2.727 | 1.913 | 2.05 | 1.886 |
| A_52_P237357 | AF095690; | | | 1.813 | 1.675 | 1.846 | 1.603 | 1.706 | 1.432 |
| A_52_P238468 | XM_133238 | 381853 | Gm160 | 2.307 | 2.173 | 2.136 | 1.929 | 1.906 | 1.683 |
| A_52_P240706 | NM_007543 | 26367 | Ceacam2 | 2.285 | 1.866 | 1.892 | 2.654 | 2.556 | 2.628 |
| A_52_P253179 | AK077477; | 16009 | Igfbp3 | 0.397 | 0.511 | 0.257 | 0.517 | 0.468 | 0.482 |
| A_52_P255224 | AK032711; XM_486482 | 234371 | BC021367 | 2.31 | 3.108 | 2.526 | 2.817 | 2.716 | 2.5 |
| A_52_P255250 | NM_009543 | 22644 | Rnf103 | 0.532 | 0.371 | 0.413 | 0.471 | 0.455 | 0.491 |
| A_52_P258116 | NM_009521 | 22415 | Wnt3 | 0.156 | 0.164 | 0.16 | 0.312 | 0.309 | 0.296 |
| A_52_P26416 | AF106279; | 16782 | | 0.254 | 0.252 | 0.241 | 0.358 | 0.391 | 0.378 |
| A_52_P269158 | NM_001003948 | 98496 | AL024069 | 0.316 | 0.33 | 0.33 | 0.388 | 0.393 | 0.443 |
| A_52_P274960 | NM_001005508 | 226652 | 6030405P05Rik | 1.83 | 1.82 | 1.948 | 2.07 | 2.181 | 2.076 |
| A_52_P277415 | AK077313; | 76252 | 0610006O14Rik | 1.282 | 1.291 | 1.224 | 0.99 | 1.044 | 0.981 |
| A_52_P282000 | AK020248; | 13800 | Enah | 2.7 | 2.435 | 2.683 | 2.632 | 2.812 | 2.59 |
| A_52_P284243 | XM_359390; XM_358896 | 384373 | LOC386546 | 0.671 | 0.371 | 0.191 | 0.435 | 0.369 | 0.372 |
| A_52_P284981 | NM_007462 | 11789 | Apc | 1.566 | 2.024 | 1.253 | 1.307 | 1.36 | 1.114 |
| A_52_P286166 | NM_001002927 | 18619 | Penk1 | 0.497 | 0.324 | 0.339 | 0.671 | 0.658 | 0.647 |
| A_52_P286528 | BC055771; | 102954 | Nudt11 | 0.411 | 0.273 | 0.263 | 0.433 | 0.371 | 0.517 |
| A_52_P286970 | XM_131162; XM_127565 | 76561 | Snx7 | 0.44 | 0.492 | 0.435 | 0.608 | 0.611 | 0.895 |
| A_52_P287576 | AK036308; | | | 1.703 | 2.271 | 1.347 | 2.425 | 2.794 | 1.7 |
| A_52_P289559 | NM_177784 | 277396 | C130068N17Rik | 0.448 | 0.222 | 0.378 | 0.393 | 0.428 | 0.506 |
| A_52_P293443 | AK087208; | 13819 | | 1.278 | 1.075 | 1.13 | 1.496 | 1.604 | 1.349 |
| A_52_P295933 | AK045060; | 14051 | B130023L16Rik | 0.336 | 0.293 | 0.594 | 0.466 | 0.5 | 0.466 |
| A_52_P296244 | BC048734; | | | 0.369 | 0.373 | 0.292 | 0.457 | 0.42 | 0.426 |
| A_52_P301821 | NM_053078 | 27528 | D0H4S114 | 0.442 | 0.427 | 0.469 | 0.472 | 0.525 | 0.486 |
| A_52_P30386 | AK084172; | | | 1.86 | 1.591 | 1.593 | 1.74 | 1.966 | 1.403 |
| A_52_P306387 | NM_178709 | 235315 | D130054N24Rik | 2.399 | 3.176 | 2.415 | 1.899 | 2.109 | 1.481 |
| A_52_P307801 | NM_007847 | 13222 | Defcr-rs2 | 0.119 | 0.091 | 0.0705 | 0.133 | 0.175 | 0.108 |
| A_52_P309718 | AK173099; | 218850 | D14Abb1e | 2.324 | 2.579 | 2.18 | 2.614 | 2.72 | 2.711 |
| A_52_P317273 | NM_172846 | 240960 | A230079K17Rik | 0.567 | 0.535 | 0.323 | 0.739 | 0.524 | 0.595 |
| A_52_P320170 | NM_144787 | 76804 | Jmjd2c | 1.626 | 2.821 | 1.963 | 2.515 | 2.362 | 2.293 |
| A_52_P322927 | XM_487182 | 435271 | LOC435271 | 2.109 | 2.45 | 2.167 | 3.906 | 4.277 | 3.453 |
| A_52_P324934 | AK045391; | 16403 | | 1.983 | 2.443 | 1.872 | 2.691 | 2.881 | 2.226 |
| A_52_P32502 | AK020822; | 77944 | A930007B11Rik | 2.593 | 4.558 | 2.534 | 2.973 | 3.231 | 2.237 |
| A_52_P3304 | NM_080446 | 117599 | Helb | 2.823 | 2.774 | 3.121 | 2.206 | 2.73 | 2.462 |
| A_52_P335089 | BE198552; | | | 1.331 | 1.348 | 1.455 | 1.151 | 1.229 | 0.977 |
| A_52_P336259 | AK087367; XM_485576 | 319467 | A930002C04Rik | 1.736 | 2.368 | 2.023 | 1.677 | 1.679 | 1.401 |
| A_52_P344424 | NM_010720 | 16891 | Lipg | 0.568 | 0.423 | 0.304 | 0.628 | 0.636 | 0.828 |
| A_52_P345626 | AY149175; | 386626 | Mzf6d | 2.677 | 3.059 | 3.002 | 3.203 | 2.748 | 3.757 |
| A_52_P34823 | NM_010747 | 17096 | Lyn | 0.232 | 0.214 | 0.173 | 0.248 | 0.256 | 0.228 |
| A_52_P350477 | NM_139295 | 193813 | Mcfd2 | 0.43 | 0.349 | 0.245 | 0.661 | 0.554 | 0.759 |
| A_52_P354363 | NM_026574 | 68142 | | 1.74 | 1.737 | 1.937 | 1.525 | 1.61 | 1.378 |
| A_52_P354785 | AK037929; | 14852 | Gspt1 | 2.114 | 1.965 | 1.835 | 1.904 | 1.907 | 2.055 |
| A_52_P355276 | NM_001002764 | 103677 | AI317223 | 2.549 | 2.993 | 2.548 | 1.703 | 1.935 | 1.511 |
| A_52_P358951 | AK081999; XM_134736 | 271457; 22688 | Rab5a | 0.417 | 0.378 | 0.448 | 0.464 | 0.449 | 0.46 |
| A_52_P359010 | AK129478; | 72193 | Sfrs2ip | 2.152 | 2.149 | 2.122 | 2.349 | 2.38 | 2.318 |
| A_52_P361534 | NM_009521 | 22415 | Wnt3 | 0.0992 | 0.0782 | 0.0774 | 0.211 | 0.198 | 0.243 |
| A_52_P362805 | AK044730; | 269254 | Als4 | 2.262 | 2.248 | 2.26 | 1.818 | 1.967 | 1.718 |
| A_52_P364021 | NM_026713 | 68393 | Mogat1 | 0.585 | 0.322 | 0.317 | 0.491 | 0.352 | 0.331 |
| A_52_P367294 | NM_176966 | 319636 | Ccdc10 | 2.167 | 2.857 | 1.874 | 1.884 | 2.063 | 1.699 |
| A_52_P3698 | NM_001015099 | 217558 | | 1.885 | 1.84 | 1.762 | 2.392 | 2.296 | 2.486 |
| A_52_P370162 | NM_175271 | 78134 | Gpr23 | 0.303 | 0.145 | 0.219 | 0.384 | 0.365 | 0.464 |
| A_52_P376169 | NM_177139 | 320343 | E130115E03Rik | 0.314 | 0.285 | 0.295 | 0.539 | 0.508 | 0.528 |
| A_52_P376502 | BC025011; | 227541 | Camk1d | 1.575 | 1.641 | 1.563 | 1.989 | 2.543 | 1.959 |
| A_52_P377299 | AK048360; | 271377 | Zbtb11 | 1.927 | 1.892 | 1.923 | 1.678 | 1.79 | 1.546 |
| A_52_P380263 | NM_013723 | 27205 | Podxl | 0.395 | 0.422 | 0.286 | 0.532 | 0.511 | 0.504 |
| A_52_P382565 | XM_483943 | 432493 | LOC432493 | 0.976 | 1.222 | 1.221 | 1.492 | 1.439 | 1.198 |
| A_52_P384392 | AK080827; | 328087 | D930001B02 | 2.365 | 2.375 | 2.393 | 1.135 | 1.269 | 1.098 |
| A_52_P384718 | BC052056; XM_283264 | 320873 | | 0.508 | 0.441 | 0.458 | 0.541 | 0.512 | 0.578 |
| A_52_P386544 | NM_026594 | 68172 | 4930517K11Rik | 2.843 | 2.618 | 2.802 | 2.673 | 2.913 | 2.894 |
| A_52_P386682 | AK033609; | 69587 | Rnf3 | 0.518 | 0.47 | 0.415 | 0.761 | 0.727 | 0.798 |
| A_52_P389874 | NM_011918 | 24131 | Ldb3 | 2.421 | 2.244 | 2.421 | 2.216 | 2.284 | 2.066 |
| A_52_P391505 | NM_144513 | 17263 | Gtl2 | 1.36 | 1.291 | 1.327 | 1.053 | 1.04 | 0.852 |
| A_52_P399095 | NM_177793 | 327747 | 9030224M15Rik | 0.366 | 0.325 | 0.163 | 0.433 | 0.401 | 0.466 |
| A_52_P400425 | NM_178656 | 193003 | A530088H08Rik | 2.498 | 2.036 | 2.006 | 2.674 | 2.834 | 2.576 |
| A_52_P402786 | NM_008935 | 19126 | Prom1 | 0.37 | 0.396 | 0.335 | 0.551 | 0.506 | 0.594 |
| A_52_P405340 | U76762; | 14158 | Fert2 | 2.538 | 2.399 | 2.414 | 2.011 | 2.124 | 1.824 |
| A_52_P410609 | NM_133685 | 106572 | 1700093E07Rik | 0.339 | 0.207 | 0.31 | 0.346 | 0.326 | 0.413 |
| A_52_P412167 | NM_080467 | 140494 | Atp6v0a4 | 2.324 | 2.449 | 2.406 | 4.276 | 4.719 | 3.334 |
| A_52_P413348 | XM_127674 | 75698 | 3110001K24Rik | 2.072 | 2.063 | 2.27 | 1.908 | 2.161 | 1.733 |
| A_52_P413661 | BC079861; XM_484162 | 217716 | Mlh3 | 2.28 | 2.065 | 2.239 | 2.466 | 2.362 | 2.456 |
| A_52_P416123 | AK020480; | 77358 | 9430069I07Rik | 2.511 | 2.833 | 2.298 | 1.785 | 1.993 | 1.656 |
| A_52_P420369 | NM_029444 | 75801 | 4930447C04Rik | 3.256 | 2.433 | 2.971 | 2.042 | 2.189 | 2.206 |
| A_52_P420608 | NM_201366 | 381371 | Gm1631 | 2.287 | 3.006 | 2.818 | 2.552 | 2.723 | 3.242 |
| A_52_P421234 | NM_133832 | 98711 | Rdh10 | 0.356 | 0.309 | 0.338 | 0.406 | 0.4 | 0.427 |
| A_52_P423384 | BC057551; XM_354546 | 104248 | Cabin1 | 1.58 | 1.963 | 1.524 | 1.442 | 1.596 | 1.426 |
| A_52_P425734 | NM_145146 | 280662 | Afm | 3.913 | 3.546 | 3.74 | 3.764 | 4.135 | 3.335 |
| A_52_P430927 | AK042385; | 11908 | Atf1 | 1.603 | 1.64 | 1.71 | 1.368 | 1.604 | 1.26 |
| A_52_P432464 | AK053863; | 192196 | Luc7l2 | 2.643 | 3.226 | 2.225 | 1.973 | 2.004 | 1.643 |
| A_52_P434549 | NM_023190 | 56215 | Acin1 | 1.868 | 1.876 | 1.953 | 1.241 | 1.454 | 1.123 |
| A_52_P438036 | NM_175271 | 78134 | Gpr23 | 0.376 | 0.334 | 0.36 | 0.455 | 0.441 | 0.489 |
| A_52_P442567 | NM_009099 | 20128 | Trim30 | 0.444 | 0.22 | 0.332 | 0.345 | 0.439 | 0.265 |
| A_52_P445799 | AK046777; | 52490 | D18Ertd289e | 0.676 | 0.602 | 0.389 | 0.613 | 0.532 | 0.592 |
| A_52_P452689 | NM_007498 | 11910 | Atf3 | 0.291 | 0.326 | 0.293 | 0.307 | 0.321 | 0.307 |
| A_52_P458916 | AK016751; | 218850 | D14Abb1e | 2.421 | 2.013 | 2.081 | 2.688 | 2.359 | 2.992 |
| A_52_P460882 | XM_130319; XM_130308 | 73668; 14725 | Ttc21b | 0.412 | 0.637 | 0.533 | 0.396 | 0.633 | 0.502 |
| A_52_P469789 | NM_007588 | 12311 | Calcr | 0.48 | 0.383 | 0.445 | 0.625 | 0.707 | 0.696 |
| A_52_P470663 | | | | 0.377 | 0.334 | 0.403 | 0.467 | 0.439 | 0.566 |
| A_52_P474242 | BE457768; | | | 0.499 | 0.53 | 0.46 | 0.69 | 0.658 | 0.726 |
| A_52_P483885 | AK078465; | 76916 | 4930455C21Rik | 2.455 | 2.834 | 1.947 | 2.313 | 2.262 | 1.788 |
| A_52_P485939 | NM_026228 | 67547 | Slc39a8 | 0.407 | 0.418 | 0.26 | 0.31 | 0.338 | 0.278 |
| A_52_P486234 | AK033497; | 22680 | | 3.81 | 2.878 | 2.669 | 3.19 | 3.59 | 3.068 |
| A_52_P487362 | NM_028980 | 74521 | 8430415E04Rik | 0.317 | 0.316 | 0.334 | 0.477 | 0.454 | 0.469 |
| A_52_P490263 | NM_013819 | 14991 | H2-M3 | 0.59 | 0.414 | 0.28 | 0.748 | 0.649 | 0.724 |
| A_52_P492189 | | | | 0.591 | 0.43 | 0.274 | 0.52 | 0.592 | 0.557 |
| A_52_P500979 | NM_013628 | 18548 | Pcsk1 | 0.75 | 0.686 | 0.598 | 1.08 | 1.031 | 0.991 |
| A_52_P501767 | AK012272; | 20637 | Snrp70 | 1.314 | 1.333 | 1.34 | 0.962 | 1 | 0.898 |
| A_52_P50496 | AY048587; XM_619453 | 114640 | Tifp39 | 0.454 | 0.501 | 0.422 | 0.661 | 0.638 | 0.694 |
| A_52_P505907 | NM_146112 | 227331 | Tnrc15 | 1.163 | 2.733 | 2.089 | 1.999 | 2.239 | 1.448 |
| A_52_P518352 | AK046930; | | | 1.712 | 1.676 | 1.27 | 1.313 | 1.441 | 1.085 |
| A_52_P519870 | AB010340; | | | 2.391 | 3.34 | 2.621 | 3.76 | 3.72 | 1.955 |
| A_52_P523399 | NM_029444 | 75801 | 4930447C04Rik | 3.312 | 7.019 | 4.341 | 3.597 | 4.009 | 2.819 |
| A_52_P532323 | NM_178410 | 218518 | Marveld2 | 0.399 | 0.326 | 0.264 | 0.434 | 0.363 | 0.431 |
| A_52_P532910 | NM_024427 | 22003 | Tpm1 | 0.358 | 0.392 | 0.35 | 0.386 | 0.39 | 0.433 |
| A_52_P537140 | AK047235; | | | 1.74 | 2.015 | 2.044 | 1.613 | 1.708 | 1.261 |
| A_52_P538515 | BC049181; | 226412 | R3hdm | 2.219 | 2.049 | 2.456 | 1.981 | 2.364 | 1.119 |
| A_52_P549815 | XM_621314 | 109620 | | 0.27 | 0.112 | 0.135 | 0.367 | 0.406 | 0.421 |
| A_52_P553100 | AK040413; | | | 2.967 | 3.025 | 3.491 | 3.19 | 3.168 | 2.79 |
| A_52_P553386 | AK041018; | | | 2.234 | 2.327 | 1.991 | 2.369 | 2.909 | 2.525 |
| A_52_P555603 | AK032770; | 11787 | Apbb2 | 1.409 | 1.222 | 1.29 | 1.326 | 1.427 | 1.18 |
| A_52_P556014 | AK087367; XM_620168 | 545738 | A930002C04Rik | 1.947 | 2.241 | 1.96 | 1.785 | 1.768 | 1.418 |
| A_52_P557293 | NM_015775 | 50528 | Tmprss2 | 0.277 | 0.205 | 0.179 | 0.45 | 0.457 | 0.355 |
| A_52_P557940 | AK004302; | 66704 | 4921506I22Rik | 2.114 | 1.863 | 2.221 | 2.17 | 2.013 | 2.051 |
| A_52_P560006 | NM_008426 | 16519 | Kcnj3 | 0.763 | 0.536 | 0.497 | 0.479 | 0.449 | 0.493 |
| A_52_P561046 | AK044509; | | | 1.501 | 1.345 | 1.04 | 1.708 | 2.08 | 1.486 |
| A_52_P565940 | AF419220; | 18193 | Nsd1 | 1.84 | 1.873 | 1.944 | 1.445 | 1.546 | 1.33 |
| A_52_P566681 | NM_153581 | 234267 | Gpm6a | 0.396 | 0.243 | 0.333 | 0.326 | 0.468 | 0.481 |
| A_52_P569375 | NM_010203 | 14176 | Fgf5 | 0.776 | 0.678 | 0.554 | 0.45 | 0.467 | 0.574 |
| A_52_P57078 | AK052455; | | | 2.172 | 1.912 | 1.757 | 1.727 | 1.952 | 1.877 |
| A_52_P571746 | XM_355533; XM_357399 | 230863 | LOC381571 | 5.455 | 5.043 | 4.955 | 4.478 | 4.627 | 3.701 |
| A_52_P571987 | AK053601; | 15525 | | 1.669 | 2.992 | 2.149 | 2.356 | 2.814 | 1.19 |
| A_52_P574668 | NM_011851 | 23959 | Nt5e | 0.15 | 0.159 | 0.148 | 0.316 | 0.285 | 0.335 |
| A_52_P575505 | NM_008994 | 19302 | Pxmp3 | 0.305 | 0.306 | 0.317 | 0.463 | 0.445 | 0.525 |
| A_52_P577072 | NM_011901 | 24074 | Taf7 | 2.198 | 2.692 | 2.144 | 1.999 | 2.035 | 1.851 |
| A_52_P581100 | NM_027285 | 70005 | 1700029I01Rik | 2.295 | 3.266 | 2.708 | 3.168 | 3.146 | 2.879 |
| A_52_P581138 | NM_212450 | 329506 | D2Ertd485e | 1.847 | 1.821 | 1.932 | 1.418 | 1.573 | 1.284 |
| A_52_P584874 | NM_152895 | 75605 | Jarid1b | 2.228 | 2.064 | 2.215 | 1.844 | 1.912 | 1.769 |
| A_52_P586157 | NM_022309 | 12400 | Cbfb | 0.481 | 0.354 | 0.384 | 0.606 | 0.523 | 0.75 |
| A_52_P59318 | NM_133832 | 98711 | Rdh10 | 0.306 | 0.273 | 0.275 | 0.373 | 0.395 | 0.445 |
| A_52_P601333 | AK078961; | 67786 | 6230424C14Rik | 0.466 | 0.125 | 0.157 | 0.36 | 0.335 | 0.333 |
| A_52_P620345 | NM_133979 | 102566 | AI604832 | 0.368 | 0.402 | 0.382 | 0.441 | 0.438 | 0.493 |
| A_52_P626069 | AK040994; XM_284439 | 109151 | Chd9 | 1.61 | 3.967 | 1.955 | 2.209 | 2.26 | 1.715 |
| A_52_P628212 | AK044339; | 26380 | | 9.353 | 5.604 | 7.112 | 6.273 | 6.769 | 7.237 |
| A_52_P630964 | NM_027533 | 70747 | Tspan2 | 0.361 | 0.283 | 0.324 | 0.499 | 0.473 | 0.525 |
| A_52_P634829 | NM_023835 | 76681 | Trim12 | 0.59 | 0.561 | 0.672 | 1.012 | 0.996 | 0.866 |
| A_52_P636199 | NM_008017 | 14211 | Smc2l1 | 1.61 | 2.123 | 1.631 | 1.531 | 1.607 | 1.38 |
| A_52_P636559 | AK053008; | 268469 | 9530033F24Rik | 2.63 | 2.537 | 2.752 | 2.132 | 2.334 | 1.998 |
| A_52_P650215 | NM_029278 | 75416 | 2610033H07Rik | 1.439 | 4.127 | 1.948 | 2.359 | 2.484 | 1.577 |
| A_52_P651469 | NM_001012726 | 503692 | | 5.11 | 4.303 | 5.085 | 5.606 | 6.363 | 4.012 |
| A_52_P654624 | AK005333; | 236604 | 4933439C20Rik | 2.049 | 1.763 | 2.06 | 1.639 | 1.865 | 1.518 |
| A_52_P655008 | AK044382; | 320678 | A930037G23Rik | 1.653 | 1.779 | 1.572 | 1.52 | 1.586 | 1.325 |
| A_52_P656336 | NM_023733 | 74114 | Crot | 0.407 | 0.351 | 0.381 | 0.755 | 0.647 | 0.8 |
| A_52_P661144 | XM_485365 | | | 2.066 | 2.37 | 2.399 | 2.3 | 2.365 | 2.13 |
| A_52_P6621 | | | | 2.585 | 4.954 | 2.769 | 4.277 | 4.565 | 2.653 |
| A_52_P672761 | NM_013689 | 21682 | Tec | 0.215 | 0.109 | NO DATA | 0.476 | 0.33 | 0.379 |
| A_52_P676403 | NM_019494 | 56066 | Cxcl11 | 0.525 | 0.596 | 0.597 | 0.761 | 0.809 | 0.704 |
| A_52_P676510 | NM_011579 | 21822 | Tgtp | 0.225 | 0.162 | 0.155 | 0.382 | 0.378 | 0.362 |
| A_52_P680436 | NM_025793 | 66840 | Wdr45l | 0.344 | 0.369 | 0.331 | 0.651 | 0.557 | 0.675 |
| A_52_P680941 | NM_010284 | 14600 | Ghr | 0.389 | 0.396 | 0.371 | 0.448 | 0.445 | 0.488 |
| A_52_P68702 | NM_145148 | 232288 | Frmd4b | 0.545 | 0.476 | 0.538 | 0.501 | 0.43 | 0.424 |
| A_52_P68942 | NM_026952 | 69131 | Crk7 | 1.741 | 2.133 | 1.422 | 1.895 | 2.138 | 1.574 |
| A_52_P731549 | AK085965; | 67939 | 2010316F05Rik | 1.602 | 1.397 | 1.252 | 1.252 | 1.393 | 1.078 |
| A_52_P74441 | NM_011570 | 21753 | Tes | 0.423 | 0.418 | 0.328 | 0.54 | 0.558 | 0.659 |
| A_52_P75971 | NM_026599 | 68178 | Cgnl1 | 1.56 | 1.535 | 1.702 | 1.265 | 1.357 | 1.222 |
| A_52_P78168 | AK031518; | | | 2.108 | 1.677 | 1.617 | 1.783 | 1.791 | 1.711 |
| A_52_P795628 | AK086352; | | | 4.861 | 5.344 | 4.508 | 6.792 | 7.236 | 5.913 |
| A_52_P804224 | | | | 1.522 | 1.688 | 1.828 | 2.31 | 2.487 | 2.241 |
| A_52_P812362 | BC016205; | 408064 | BC064078 | 0.25 | 0.167 | 0.217 | 0.302 | 0.27 | 0.27 |
| A_52_P81252 | NM_172599 | 218978 | D14Ertd436e | 2.478 | 2.667 | 2.607 | 1.82 | 1.981 | 1.764 |
| A_52_P819356 | AK041684; | | | 3.003 | 2.848 | 2.7 | 3.026 | 2.915 | 3.006 |
| A_52_P82488 | NM_021476 | 58861 | Cysltr1 | 0.319 | 0.379 | 0.306 | 0.5 | 0.438 | 0.534 |
| A_52_P826666 | AK007019; | 51799 | Rap2ip | 4.063 | 3.142 | 3.547 | 3.365 | 3.764 | 3.267 |
| A_52_P82722 | NM_009988 | 13052 | Cxadr | 0.311 | 0.136 | 0.21 | 0.361 | 0.254 | 0.355 |
| A_52_P827911 | AK081941; | | | 2.387 | 2.657 | 2.47 | 2.075 | 2.44 | 2.116 |
| A_52_P84015 | AK037762; | 11863 | | 3.675 | 3.344 | 3.705 | 4.008 | 3.981 | 3.736 |
| A_52_P85805 | NM_009525 | 22419 | Wnt5b | 0.548 | 0.377 | 0.209 | 0.495 | 0.38 | 0.469 |
| A_52_P87839 | NM_013657 | 20348 | Sema3c | 0.32 | 0.145 | 0.267 | 0.494 | 0.379 | 0.47 |
| A_52_P93837 | NM_008604 | 17380 | Mme | 0.559 | 0.439 | 0.513 | 0.653 | 0.573 | 0.747 |
| A_52_P947879 | AK048751; | | | 1.687 | 1.729 | 1.647 | 2.149 | 2.232 | 2.009 |
| A_52_P957500 | XM_622354 | | | 2.438 | 1.963 | 2.267 | 2.142 | 2.231 | 1.855 |
| A_52_P995717 | AK045222; | | | 3.292 | 3.495 | 3.303 | 2.04 | 2.108 | 1.729 |

TABLE 1B
Training Set Genes (Non Gemline Competent)

| | | | | Clone 1 | | | Clone 2 | | |
|---|---|---|---|---|---|---|---|---|---|
| Agilent Probe ID | Genbank | Entrez ID | Gene Symbol | rep. 1 | rep. 2 | rep. 3 | rep. 1 | rep. 2 | rep. 3 |
| A_51_P101975 | NM_010205 | 14179 | Fgf8 | 2.683 | 2.96 | 2.139 | 3.25 | 3.463 | 3.854 |
| A_51_P102081 | NM_007847 | 13222 | Defcr-rs2 | 2.408 | 2.238 | 1.498 | 3.38 | 3.255 | 2.763 |
| A_51_P102257 | AK053112; XM_619640 | 320477 | E030037J05Rik | 0.455 | 0.507 | 0.624 | 1.76 | 1.58 | 1.703 |
| A_51_P103397 | AK078521; | 54561 | Nap1l3 | 0.942 | 0.996 | 1.004 | 1.047 | 0.978 | 1.076 |
| A_51_P103406 | NM_011708 | 22371 | Vwf | 0.798 | 1.058 | 1.093 | 1.007 | 0.932 | 1.019 |
| A_51_P104162 | AK053379; | 328232 | | 0.351 | 0.468 | 0.481 | 1.289 | 1.159 | 1.082 |
| A_51_P105068 | NM_027990 | 71897 | 2310010M24Rik | 1.902 | 1.982 | 1.72 | 2.012 | 2.165 | 2.002 |
| A_51_P106918 | NM_009051 | 19715 | Rex2 | 1.032 | 1.019 | 0.654 | 1.31 | 1.285 | 1.541 |
| A_51_P109649 | AK018142; | 17263 | Gtl2 | 0.11 | 0.17 | 0.136 | 0.167 | 0.167 | 0.195 |
| A_51_P113162 | AK086843; | | | 0.616 | 0.947 | 0.722 | 1.731 | 1.215 | 1.78 |
| A_51_P114606 | NM_016965 | 50884 | Nckap1 | 1.512 | 1.408 | 1.677 | 0.736 | 0.694 | 0.805 |
| A_51_P115005 | NM_010104 | 13614 | Edn1 | 0.521 | 0.469 | 0.61 | 0.968 | 0.997 | 1.054 |
| A_51_P121635 | NM_030700 | 80884 | Maged2 | 1.225 | 1.426 | 1.335 | 0.918 | 0.99 | 0.88 |
| A_51_P124315 | NM_177649 | 227712 | BC034076 | 0.654 | 0.611 | 0.726 | 1.267 | 1.174 | 1.278 |
| A_51_P125679 | AK010725; XM_134948 | 73673 | 2410076I21Rik | 0.588 | 0.959 | 0.916 | 0.84 | 0.843 | 0.849 |
| A_51_P127560 | AK051724; | 76577 | | 0.24 | 0.497 | 0.587 | 1.321 | 1.349 | 1.505 |
| A_51_P130095 | NM_010187 | 14130 | Fcgr2b | 0.4 | 0.349 | 0.423 | 0.624 | 0.547 | 0.453 |
| A_51_P140751 | NM_011397 | 20522 | Slc23a1 | 0.522 | 0.618 | 0.556 | 1.152 | 1.255 | 1.186 |
| A_51_P141660 | AK053665; XM_358382 | 67776; 235380 | Loh11cr2a | 0.43 | 0.62 | 0.908 | 1.844 | 1.422 | 1.976 |
| A_51_P144417 | NM_181750 | 226412 | R3hdm | 0.745 | 0.757 | 0.789 | 2.029 | 1.993 | 2.191 |
| A_51_P144712 | NM_178930 | 107338 | Gbf1 | 0.424 | 0.476 | 0.425 | 1.192 | 1.19 | 1.292 |
| A_51_P147651 | AK041797; XM_146705 | 245902 | | 0.511 | 0.729 | 0.963 | 1.47 | 1.214 | 1.502 |
| A_51_P149469 | NM_009608 | 11464 | Actc1 | 1.012 | 1.006 | 0.935 | 0.971 | 0.997 | 1.12 |
| A_51_P153079 | NM_133923 | 101100 | 4833441J24Rik | 0.42 | 0.232 | 0.278 | 1.153 | 1.158 | 1.047 |
| A_51_P157986 | NM_029930 | 77574 | 3321401G04Rik | 0.909 | 1.196 | 1.088 | 0.563 | 0.621 | 0.598 |
| A_51_P158037 | NM_013505 | 13506 | Dsc2 | 1.238 | 1.562 | 1.304 | 0.743 | 0.819 | 0.875 |
| A_51_P160160 | AK017624; | 70548 | 5730437C12Rik | 1.025 | 1.558 | 1.042 | 0.701 | 0.738 | 0.923 |
| A_51_P161115 | AK050756; | | | NO DATA | 0.189 | 0.363 | 1.733 | 0.972 | 1.474 |
| A_51_P162474 | AF285577; | 107815 | Scml2 | 2.885 | 2.549 | 2.371 | 3.021 | 3.116 | 3.1 |
| A_51_P162481 | NM_133194 | 107815 | Scml2 | 2.434 | 2.616 | 2.232 | 2.312 | 2.394 | 2.596 |
| A_51_P164219 | NM_011909 | 24110 | Usp18 | 1.876 | 1.698 | 1.64 | 1.445 | 1.463 | 1.349 |
| A_51_P164504 | NM_007469 | 11812 | Apoc1 | 1.307 | 1.245 | 1.314 | 1.412 | 1.375 | 1.457 |
| A_51_P165082 | AB041601; XM_489523 | 224019 | D16Bwg1494e | 1.656 | 1.924 | 1.93 | 1.376 | 1.394 | 1.295 |
| A_51_P165244 | NM_018734 | 55932 | Gbp4 | 2.388 | 1.319 | 2.543 | 2.404 | 2.051 | 2.534 |
| A_51_P168646 | AK039580; | | | 1.655 | 1.373 | 1.436 | 2.357 | 2.478 | 2.267 |
| A_51_P171728 | NM_011926 | 26365 | Ceacam1 | 0.474 | 0.703 | 0.686 | 0.748 | 0.952 | 0.943 |
| A_51_P174943 | NM_008485 | 16782 | Lamc2 | 1.282 | 1.255 | 1.249 | 1.068 | 1.04 | 1.004 |
| A_51_P176474 | AK010466; | 76484 | 2410012C07Rik | 0.842 | 0.903 | 1.004 | 0.996 | 1.036 | 1.127 |
| A_51_P180413 | NM_176930 | 319504 | C130076O07Rik | 1.603 | 1.54 | 1.395 | 1.297 | 1.25 | 1.155 |
| A_51_P187645 | BC004786; XM_354700 | 16061 | Igh-VJ558 | 2.489 | 2.63 | 2.11 | 2.267 | 1.973 | 2.108 |
| A_51_P189631 | NM_172427 | 76455 | 2310067E19Rik | 0.318 | 0.341 | 0.373 | 1.015 | 0.905 | 0.892 |
| A_51_P191990 | AK087548; | 26563 | Ror1 | 0.378 | 0.368 | 0.389 | 1.197 | 1.149 | 1.176 |
| A_51_P193019 | NM_011157 | 19073 | Prg1 | 0.263 | 0.419 | 0.589 | 1.602 | 0.893 | 0.295 |
| A_51_P199888 | NM_026082 | 67299 | Dock7 | 1.182 | 1.246 | 1.295 | 0.669 | 0.678 | 1 |
| A_51_P200223 | XM_619497 | | | 0.65 | 0.646 | 0.76 | 1.392 | 1.367 | 1.332 |
| A_51_P202911 | NM_144882 | 67198 | 2810022L02Rik | 1.005 | 0.995 | 0.908 | 0.913 | 0.931 | 0.902 |
| A_51_P203955 | NM_010260 | 14469 | Gbp2 | 2.194 | 2.061 | 2.214 | 2.45 | 2.681 | 2.345 |
| A_51_P207934 | NM_199016 | 224794 | Enpp4 | 0.71 | 0.576 | 0.811 | 1.068 | 0.833 | 1.143 |
| A_51_P219166 | NM_027756 | 71306 | Mfap3l | 1.187 | 1.279 | 1.37 | 1.013 | 0.915 | 1.095 |
| A_51_P221291 | NM_009229 | 20650 | Sntb2 | 0.614 | 0.612 | 0.631 | 1.259 | 1.1 | 1.157 |
| A_51_P222773 | NM_010446 | 15376 | Foxa2 | 2.333 | 1.693 | 1.64 | 6.867 | 6.289 | 6.137 |
| A_51_P223132 | NM_031183; XM_486227 | 83395; 70549 | Sp6 | 0.649 | 0.542 | 0.552 | 1.095 | 0.998 | 1.125 |
| A_51_P223656 | NM_008484 | 16780 | Lamb3 | 0.994 | 1.434 | 1.253 | 1.02 | 1.017 | 1.132 |
| A_51_P228472 | NM_008343 | 16009 | Igfbp3 | 1.472 | 2.26 | 1.637 | 1.02 | 1.11 | 1.239 |
| A_51_P229613 | NM_008792 | 18549 | Pcsk2 | 1.463 | 1.53 | 1.37 | 1.333 | 1.422 | 1.554 |
| A_51_P231341 | AK087764; | 108160 | D0HXS9928E | 0.131 | 0.284 | 0.334 | 1.584 | 1 | 1.654 |
| A_51_P236267 | NM_009183 | 20452 | St8sia4 | 1.305 | 1.382 | 1.289 | 1.168 | 1.234 | 1.322 |
| A_51_P237040 | NM_008711 | 18121 | Nog | 1.152 | 1.407 | 1.101 | 0.859 | 0.96 | 0.924 |
| A_51_P240253 | NM_019662 | 56437 | Rrad | 1.392 | 1.648 | 1.29 | 0.851 | 0.9 | 0.913 |
| A_51_P240811 | NM_009290 | 20890 | Wnt8a | 4.437 | 4.432 | 3.479 | 4.373 | 5.655 | 4.468 |
| A_51_P242399 | NM_031170 | 16691 | Krt2-8 | 1.013 | 1.199 | 0.939 | 0.962 | 0.977 | 0.946 |
| A_51_P252565 | NM_009660 | 11687 | Alox15 | 1.618 | 1.425 | 1.194 | 1.366 | 1.436 | 1.457 |
| A_51_P253053 | XM_618867 | | | 0.601 | 1.748 | 0.724 | 1.594 | 1.644 | 2.037 |
| A_51_P262171 | NM_008326 | 15944 | Ifi1 | 1.271 | 1.011 | 1.135 | 1.457 | 1.571 | 1.485 |
| A_51_P266403 | AK009163; | 76730 | 2310005C01Rik | 0.984 | 0.901 | 1.009 | 1.273 | 1.313 | 1.391 |
| A_51_P266644 | BC055753; XM_125538 | 140742 | | 1.51 | 1.548 | 1.621 | 1.064 | 0.96 | 1.069 |
| A_51_P268167 | BC037232; XM_621365 | 229363; 218850 | Gmps | 0.465 | 0.448 | 0.523 | 1.267 | 1.053 | 1.205 |
| A_51_P270725 | AK089493; | 102093 | Phkb | 0.756 | 0.923 | 0.755 | 2.239 | 1.725 | 2.287 |
| A_51_P272363 | NM_134160 | 171166 | Mcoln3 | 2.423 | 3.074 | 2.25 | 1.242 | 1.432 | 0.843 |
| A_51_P275435 | NM_172411 | 71874 | 2310007B03Rik | 3.355 | 3.105 | 3.53 | 5.636 | 6.155 | 6.288 |
| A_51_P276305 | NM_009292 | 20899 | Stra8 | 0.873 | 0.934 | 0.982 | 0.431 | 0.426 | 0.471 |
| A_51_P282227 | NM_020577 | 57344 | As3mt | 0.79 | 0.761 | 0.777 | 1.02 | 0.91 | 0.98 |
| A_51_P283300 | NM_023116 | 12296 | Cacnb2 | 1.159 | 1.029 | 1.146 | 0.648 | 0.72 | 0.636 |
| A_51_P285725 | NM_009309 | 20997 | T | 2.672 | 1.6 | 1.512 | 3.129 | 3.214 | 3.267 |
| A_51_P289341 | AK030946; XM_141372 | 241639 | 5830467J12Rik | 0.978 | 1.482 | 1.063 | 0.881 | 0.972 | 1.046 |
| A_51_P291227 | NM_001005508 | 226652 | 6030405P05Rik | 0.705 | 0.681 | 0.731 | 0.814 | 0.735 | 0.798 |
| A_51_P294505 | NM_025658 | 66607 | Ms4a4d | 1.546 | 1.551 | 1.883 | 2.127 | 2.066 | 2.183 |
| A_51_P294566 | AK020687; | 78083 | 9930116N10Rik | 0.689 | 1.237 | 0.848 | 1.59 | 1.475 | 1.545 |
| A_51_P297131 | NM_009502 | 22330 | Vcl | 1.015 | 1.436 | 0.955 | 0.556 | 0.672 | 0.587 |
| A_51_P298266 | NM_025681 | 66643 | Lix1 | 1.123 | 1.145 | 1.109 | 1.143 | 1.15 | 1.141 |
| A_51_P302377 | NM_008009 | 14181 | Fgfbp1 | 1.542 | 1.782 | 1.567 | 1.002 | 1.052 | 1.132 |
| A_51_P305019 | AK020601; | 78423 | 9530049O05Rik | 0.201 | 0.4 | 0.429 | 1.187 | 1.059 | 1.249 |
| A_51_P311038 | BC009666; XM_134720 | 57267 | Apba3 | 1.847 | 1.642 | 1.538 | 2.55 | 2.405 | 2.808 |
| A_51_P316733 | AK173008; | 330914 | Grit | 0.195 | 0.471 | 0.456 | 1.1 | 1.238 | 1.129 |
| A_51_P318577 | AK010729; | 73671 | 2410078J06Rik | 0.605 | 0.863 | 0.641 | 0.897 | 1.028 | 1.15 |
| A_51_P321579 | XM_131865; XM_196334 | 433809 | LOC433809 | 0.495 | 0.534 | 0.598 | 1.155 | 1.087 | 1.237 |
| A_51_P327011 | NM_016744 | 18573 | Pde1a | 1.629 | 1.061 | 1.167 | 1.387 | 1.253 | 1.051 |
| A_51_P330380 | XM_130138; XM_145466 | 243881 | 4921517N04Rik | 0.714 | 1.211 | 0.583 | 1.659 | 2.149 | 0.895 |
| A_51_P333253 | NM_178440 | 246177 | Myo1g | 0.535 | 0.491 | 0.538 | 0.474 | 0.481 | 0.37 |
| A_51_P334093 | NM_026228 | 67547 | Slc39a8 | 2.173 | 2.638 | 2.466 | 1.07 | 1.006 | 1.061 |
| A_51_P335460 | NM_009132 | 20259 | Scin | 1.658 | 1.913 | 1.519 | 1.101 | 1.225 | 1.107 |
| A_51_P337125 | NM_010566 | 16331 | Inpp5d | 0.938 | 0.873 | 1.036 | 1.261 | 1.272 | 1.297 |
| A_51_P341137 | AK035176; | 224829 | Trerf1 | 0.563 | 0.731 | 0.649 | 1.51 | 1.436 | 1.382 |
| A_51_P343129 | AK012079; | 70472 | Atad2 | 0.633 | 0.896 | 0.848 | 1.965 | 1.637 | 1.677 |
| A_51_P345422 | NM_010747 | 17096 | Lyn | 0.888 | 0.803 | 0.881 | 1.006 | 0.995 | 0.89 |
| A_51_P352572 | NM_019701 | 56365 | Clcnkb | 1.441 | 1.565 | 1.422 | 1.707 | 1.362 | 1.314 |
| A_51_P353221 | NM_011582 | 21828 | Thbs4 | 1.037 | 0.982 | 1.145 | 0.826 | 0.851 | 0.782 |
| A_51_P354265 | NM_009256 | 20723 | Serpinb9 | 1.028 | 1.006 | 1.064 | 0.948 | 0.995 | 0.916 |
| A_51_P354307 | NM_172469 | 209195 | Clic6 | 1.31 | 1.578 | 1.57 | 0.719 | 0.748 | 0.659 |
| A_51_P356003 | NM_001007580 | 333564 | Gm784 | 1.223 | 1.221 | 1.186 | 1.543 | 1.618 | 1.409 |
| A_51_P356883 | NM_008728 | 18162 | Npr3 | 0.813 | 0.973 | 1.027 | 0.692 | 0.599 | 0.672 |
| A_51_P358344 | NM_007732 | 12821 | Col17a1 | 0.283 | 0.661 | 0.876 | 1.118 | 0.984 | 1.246 |
| A_51_P358714 | AK031433; | | | 0.23 | 0.294 | 0.418 | 1.005 | 0.775 | 1.214 |
| A_51_P378051 | NM_009675 | 11754 | Aoc3 | 1.41 | 1.106 | 1.668 | 0.9 | 0.88 | 1.25 |
| A_51_P378371 | | | | 0.986 | 1.006 | 1.208 | 1.217 | 1.156 | 1.234 |
| A_51_P381069 | XM_132045 | 231252 | Chrna9 | 0.803 | 0.789 | 0.615 | 1.017 | 1.052 | 0.983 |
| A_51_P387528 | AK019664; | 78801 | Ak7 | 0.65 | 0.537 | 0.813 | 0.767 | 0.778 | 1.11 |
| A_51_P393518 | BC016539; | 240793 | | 1.037 | 1.122 | 1.147 | 0.727 | 0.85 | 0.8 |
| A_51_P397003 | XM_133990 | 68904 | 1110065L07Rik | 1.235 | 1.343 | 1.238 | 0.778 | 0.763 | 0.855 |
| A_51_P398766 | NM_010259 | 14468 | Gbp1 | 3.558 | 2.476 | 3.155 | 4.099 | 3.953 | 3.971 |
| A_51_P404193 | NM_022435 | 64406 | Sp5 | 1.798 | 1.335 | 1.388 | 3.365 | 3.261 | 3.306 |
| A_51_P405375 | NM_175358 | 108672 | Zdhhc15 | 1.724 | 1.355 | 1.63 | 0.826 | 1.108 | 1.317 |
| A_51_P407323 | NM_007976 | 14067 | F5 | 0.837 | 0.684 | 0.686 | 2.154 | 2.023 | 1.925 |
| A_51_P410813 | AK079732; | | | 0.717 | 0.877 | 0.904 | 1.593 | 1.473 | 1.607 |
| A_51_P412817 | NM_177617 | 216851 | D330014H01Rik | 0.725 | 0.834 | 1.04 | 1.083 | 0.986 | 0.997 |
| A_51_P418029 | NM_175541 | 245631 | Mum1l1 | 2.026 | 1.803 | 2.159 | 1.492 | 1.654 | 1.714 |
| A_51_P418375 | NM_023844 | 67374 | Jam2 | 1.044 | 0.943 | 0.99 | 0.826 | 0.786 | 0.84 |
| A_51_P418884 | NM_001013789 | 432823 | | 0.496 | 0.536 | 0.695 | 1.716 | 1.402 | 1.593 |
| A_51_P423859 | NM_007601 | 12335 | Capn3 | 0.976 | 0.768 | 0.966 | 0.825 | 0.789 | 1.053 |
| A_51_P437050 | BC072664; | 233328 | Lrrk1 | 0.941 | 1.002 | 0.944 | 1.031 | 1.066 | 0.969 |
| A_51_P442894 | AK048310; | 621603 | C130048D07Rik | 1.023 | 0.841 | 1.218 | 0.788 | 0.867 | 0.977 |
| A_51_P447189 | AK041326; XM_358090 | 385154 | | 0.755 | 0.808 | 0.675 | 2.409 | 2.277 | 2.233 |
| A_51_P455647 | NM_009801 | 12349 | Car2 | 1.592 | 1.463 | 1.337 | 2.139 | 2.347 | 2.17 |
| A_51_P455997 | Y13832; | 17263 | Gtl2 | 0.123 | 0.184 | 0.114 | 0.213 | 0.197 | 0.258 |
| A_51_P457187 | NM_028075 | 72049 | Tnfrsf13c | 1.083 | 1.58 | 1.373 | 0.916 | 1.099 | 1.091 |
| A_51_P463789 | BC051947; | 58212 | | 0.924 | 0.962 | 0.995 | 1.518 | 1.581 | 1.46 |
| A_51_P463791 | BC051947; | 58212 | | 0.648 | 0.879 | 1.013 | 1.447 | 1.472 | 1.498 |
| A_51_P463846 | NM_145545 | 229900 | 9830147J24Rik | 2.28 | 2.199 | 2.49 | 2.068 | 1.958 | 1.72 |
| A_51_P467505 | NM_013689 | 21682 | Tec | 1.077 | 1.062 | 1.153 | 0.842 | 0.861 | 0.881 |
| A_51_P473953 | XM_358600; XM_355454 | 381498 | 4930592C13Rik | 2.138 | 1.593 | 1.947 | 2.323 | 2.226 | 2.26 |
| A_51_P478930 | AK080016; | | | 0.64 | 0.623 | 0.816 | 1.097 | 1.039 | 1.17 |
| A_51_P480311 | NM_010168 | 14061 | F2 | 0.348 | 0.476 | 0.622 | 0.996 | 1.004 | 1.186 |
| A_51_P481777 | NM_018756 | 54382 | Tcstv1 | 0.208 | 0.696 | 0.654 | 0.592 | 0.503 | 0.72 |
| A_51_P481958 | NM_016674 | 12737 | Cldn1 | 2.017 | 2.213 | 2.426 | 0.904 | 0.646 | 0.974 |
| A_51_P485985 | NM_134163 | 171170 | Mbnl3 | 1.781 | 1.802 | 1.648 | 0.929 | 1.067 | 1.035 |
| A_51_P487004 | NM_027732 | 71241 | Dmrtc2 | 1.918 | 2.991 | 1.94 | 1.77 | 1.537 | 1.901 |
| A_51_P493682 | XM_134870; XM_140758 | 72672 | Rasl12 | 0.352 | 0.801 | 0.945 | 1.611 | 1.596 | 1.849 |
| A_51_P497100 | NM_010706 | 16855 | Lgals4 | 0.692 | 0.771 | 0.849 | 1.002 | 0.908 | 0.828 |
| A_51_P499854 | NM_010284 | 14600 | Ghr | 1.55 | 1.19 | 1.486 | 2.502 | 2.537 | 2.196 |
| A_51_P507880 | NM_028980 | 74521 | 8430415E04Rik | 2.104 | 2.169 | 2.081 | 2.44 | 2.499 | 2.391 |
| A_51_P512085 | NM_130449 | 140792 | Colec12 | 1.574 | 1.25 | 1.471 | 1.463 | 1.651 | 1.693 |
| A_51_P512172 | AK010953; XM_125673 | 52463 | Cxxc6 | 0.8 | 0.986 | 0.748 | 1.033 | 1.014 | 1.046 |
| A_51_P514177 | NM_028838 | 74249 | Lrrc2 | 0.911 | 0.956 | 0.975 | 1.199 | 1.209 | 1.304 |
| A_51_P520175 | AK076272; | 12223 | Btc | 0.978 | 0.996 | 1.252 | 1.035 | 0.896 | 0.744 |
| A_51_P521084 | NM_025626 | 66540 | 3110001A13Rik | 1.423 | 1.425 | 1.474 | 0.832 | 0.865 | 0.776 |
| A_52_P100042 | AK045143; | 320938 | Tnpo3 | 0.301 | 0.507 | 0.406 | 1.444 | 1.417 | 1.439 |
| A_52_P1011737 | AK039096; | 106258 | AI790442 | 0.224 | 0.44 | 0.438 | 1.367 | 1.308 | 1.503 |
| A_52_P1013441 | | | | 1.255 | 1.032 | 2.041 | 1.173 | 1.203 | 1.341 |
| A_52_P103929 | NM_009306 | 20979 | Syt1 | 0.898 | 0.938 | 0.967 | 1.626 | 1.464 | 1.422 |
| A_52_P1044262 | AK051949; | | | 0.374 | 0.535 | 0.443 | 1.042 | 1.044 | 1.091 |
| A_52_P1044696 | AK087281; | | | 0.323 | 0.722 | 0.629 | 1.236 | 1.397 | 1.547 |
| A_52_P1075773 | AK036921; | 399602 | 9930024M15Rik | 0.274 | 0.379 | 0.59 | 1.459 | 1.225 | 1.486 |
| A_52_P1084149 | AK051413; | 553138 | 9930021J03Rik | 0.447 | 0.508 | 0.452 | 1.499 | 1.58 | 1.485 |
| A_52_P1108139 | AK053079; | | | 0.169 | 0.348 | 0.199 | 1.068 | 1.372 | 1.161 |
| A_52_P1124598 | AK081090; | | | 0.304 | 0.598 | 0.609 | 1.045 | 0.474 | 1.511 |
| A_52_P113190 | NM_053214 | 17916 | Myo1f | 0.386 | 0.513 | 0.397 | 0.243 | 0.232 | 0.264 |
| A_52_P1172548 | AK084146; | 13204 | Dhx15 | 0.655 | 0.839 | 0.889 | 2.469 | 2.249 | 2.786 |
| A_52_P1180315 | AK049088; | 74022 | 3930401K13Rik | 0.255 | 0.505 | 0.516 | 1.337 | 1.179 | 0.966 |
| A_52_P1188270 | AK042362; | | | 0.341 | 0.594 | 0.564 | 1.758 | 1.462 | 1.658 |
| A_52_P1196751 | AK086488; | | | 0.314 | 0.63 | 0.521 | 1.285 | 1.098 | 1.263 |
| A_52_P126158 | NM_008326 | 15944 | Ifi1 | 1.157 | 1.034 | 1.006 | 1.254 | 1.3 | 1.232 |
| A_52_P129428 | BC049774; | 57265 | Fzd2 | 1.029 | 0.862 | 1.274 | 1.343 | 1.356 | 1.851 |
| A_52_P132608 | NM_172685 | 229731 | Slc25a24 | 0.809 | 0.906 | 0.807 | 0.708 | 0.722 | 0.693 |
| A_52_P135873 | XM_132015; XM_488584 | 231128 | BC037112 | 0.389 | 0.408 | 0.392 | 1.158 | 1.118 | 1.166 |
| A_52_P13815 | NM_010686 | 16792 | Laptm5 | 0.187 | 0.227 | 0.332 | 0.483 | 0.494 | 0.599 |
| A_52_P147870 | NM_029963 | 77721 | Mrps5 | 0.574 | 0.622 | 0.64 | 1.321 | 1.377 | 1.297 |
| A_52_P148222 | BC055037; | 224139 | 4930428L02Rik | 1.242 | 1.264 | 1.274 | 1.061 | 0.874 | 0.976 |
| A_52_P152983 | AK011739; | 26886 | Cenph | 0.423 | 0.805 | 0.28 | 1.393 | 1.582 | 1.833 |
| A_52_P153685 | AK129323; | 70422 | 2810417D08Rik | 1.763 | 1.676 | 1.845 | 2.204 | 2.099 | 2.085 |
| A_52_P159095 | AK051960; | 17685 | | 0.49 | 0.948 | 0.865 | 2.32 | 2.123 | 2.226 |
| A_52_P163148 | AB010370; | 544945 | | 0.704 | 0.687 | 0.76 | 1.098 | 1.144 | 1.325 |
| A_52_P164524 | NM_013628 | 18548 | Pcsk1 | 2.458 | 2.533 | 2.589 | 2.384 | 2.201 | 2.401 |
| A_52_P165497 | NM_011385 | 20481 | Ski | 0.541 | 0.665 | 0.649 | 1.004 | 0.817 | 0.972 |
| A_52_P165610 | NM_153534 | 210044 | Adcy2 | 1.106 | 1.129 | 1.34 | 0.778 | 0.709 | 0.763 |
| A_52_P16973 | AK081934; | 320391 | D930017J03Rik | 0.56 | 0.604 | 0.612 | 1.894 | 1.918 | 1.856 |
| A_52_P175685 | NM_008390 | 16362 | Irf1 | 1.072 | 1.33 | 1.162 | 0.971 | 1.106 | 1.017 |
| A_52_P176333 | NM_001005787 | 12695 | Cipp | 1.168 | 1.146 | 1.249 | 0.974 | 1.014 | 1.036 |
| A_52_P179250 | AK129127; | 230249 | AI314180 | 0.641 | 0.606 | 0.56 | 3.467 | 4.289 | 6.052 |
| A_52_P183368 | NM_011535 | 21386 | Tbx3 | 0.707 | 0.655 | 0.649 | 0.948 | 0.84 | 0.841 |
| A_52_P18887 | NM_011468 | 20755 | Sprr2a | 7.008 | 6.235 | 5.038 | 6.207 | 6.689 | 6.331 |
| A_52_P196568 | NM_144513 | 17263 | Gtl2 | 0.0697 | 0.0369 | 0.0417 | 0.112 | 0.0801 | 0.115 |
| A_52_P197826 | NM_010397 | 15039 | H2-T22 | 1.502 | 1.687 | 1.444 | 1.028 | 1.049 | 1.039 |
| A_52_P199633 | NM_199146 | 209387 | LOC209387 | 0.911 | 1.276 | 1.628 | 1.284 | 1.253 | 2.216 |
| A_52_P199922 | | | | 0.316 | 0.331 | 0.255 | 1.479 | 1.195 | 1.071 |
| A_52_P200458 | NM_010242 | 14345 | Fut4 | 1.003 | 0.997 | 1.017 | 1.108 | 1.203 | 1.09 |
| A_52_P206573 | NM_027427 | 70439 | Taf15 | 0.525 | 0.7 | 0.806 | 1.685 | 1.521 | 1.76 |
| A_52_P211956 | NM_025658 | 66607 | Ms4a4d | 1.71 | 1.799 | 1.851 | 2.105 | 2.274 | 1.961 |
| A_52_P214042 | NM_028125 | 72147 | Btbd4 | 1.267 | 1.407 | 1.626 | 0.852 | 0.647 | 0.866 |
| A_52_P21595 | NM_028238 | 72433 | Rab38 | 1.043 | 0.791 | 0.992 | 0.807 | 0.819 | 0.929 |
| A_52_P21628 | NM_019978 | 13175 | Dcamkl1 | 1.407 | 1.646 | 1.591 | 1.067 | 1.203 | 1.097 |
| A_52_P223083 | BC038323; | 72096 | 2010208K18Rik | 0.89 | 0.64 | 1.018 | 2.024 | 1.641 | 2.309 |
| A_52_P224125 | XM_622260 | | | 0.593 | 0.637 | 0.657 | 1.232 | 0.927 | 0.967 |
| A_52_P233673 | NM_020594 | 57432 | Zc3hdc8 | 0.731 | 0.498 | 0.877 | 1.204 | 1.041 | 1.395 |
| A_52_P237357 | AF095690; | | | 0.454 | 0.422 | 0.442 | 1.34 | 1.372 | 1.159 |
| A_52_P238468 | XM_133238 | 381853 | Gm160 | 0.673 | 0.817 | 0.882 | 1.027 | 1.116 | 1.106 |
| A_52_P240706 | NM_007543 | 26367 | Ceacam2 | 0.87 | 0.778 | 0.892 | 0.689 | 0.756 | 0.859 |
| A_52_P253179 | AK077477; | 16009 | Igfbp3 | 2.157 | 1.536 | 2.035 | 1.993 | 1.965 | 2.545 |
| A_52_P255224 | AK032711; XM_486482 | 234371 | BC021367 | 0.812 | 0.712 | 0.548 | 2.288 | 1.957 | 2.287 |
| A_52_P255250 | NM_009543 | 22644 | Rnf103 | 1.205 | 1.217 | 1.524 | 0.663 | 0.701 | 0.687 |
| A_52_P258116 | NM_009521 | 22415 | Wnt3 | 1.304 | 1.249 | 1.083 | 1.811 | 1.943 | 2.006 |
| A_52_P26416 | AF106279; | 16782 | | 1.436 | 1.802 | 1.779 | 0.723 | 0.806 | 0.697 |
| A_52_P269158 | NM_001003948 | 98496 | AL024069 | 1.775 | 1.419 | 1.506 | 1.494 | 1.391 | 1.254 |
| A_52_P274960 | NM_001005508 | 226652 | 6030405P05Rik | 0.671 | 0.634 | 0.712 | 0.763 | 0.781 | 0.808 |
| A_52_P277415 | AK077313; | 76252 | 0610006O14Rik | 0.0165 | 0.0277 | 0.0223 | 0.0497 | 0.0428 | 0.0552 |
| A_52_P282000 | AK020248; | 13800 | Enah | 0.368 | 0.482 | 0.376 | 1.791 | 1.63 | 1.536 |
| A_52_P284243 | XM_359390; XM_358896 | 384373 | LOC386546 | 1.151 | 1.169 | 1.447 | 0.803 | 0.82 | 0.911 |
| A_52_P284981 | NM_007462 | 11789 | Apc | 0.319 | 0.415 | 0.649 | 1.173 | 0.969 | 1.155 |
| A_52_P286166 | NM_001002927 | 18619 | Penk1 | 1.649 | 2.509 | 2.439 | 1.542 | 1.453 | 1.374 |
| A_52_P286528 | BC055771; | 102954 | Nudt11 | 1.419 | 1.344 | 1.431 | 1.048 | 1.063 | 1.053 |
| A_52_P286970 | XM_131162; XM_127565 | 76561 | Snx7 | 1.591 | 2.536 | 1.327 | 1.128 | 1.271 | 0.96 |
| A_52_P287576 | AK036308; | | | 0.284 | 0.537 | 0.429 | 1.515 | 1.516 | 1.834 |
| A_52_P289559 | NM_177784 | 277396 | C130068N17Rik | 0.89 | 1.215 | 1.34 | 0.528 | 0.7 | 0.835 |
| A_52_P293443 | AK087208; | 13819 | | 0.179 | 0.222 | 0.219 | 0.543 | 0.565 | 0.552 |
| A_52_P295933 | AK045060; | 14051 | B130023L16Rik | 1.637 | 1.973 | 2.08 | 1.007 | 0.993 | 1.019 |
| A_52_P296244 | BC048734; | | | 2.333 | 2.799 | 1.851 | 2.371 | 2.371 | 2.372 |
| A_52_P301821 | NM_053078 | 27528 | D0H4S114 | 1.076 | 1.275 | 1.138 | 0.88 | 0.828 | 0.741 |
| A_52_P30386 | AK084172; | | | 0.381 | 0.53 | 0.593 | 1.296 | 1.275 | 1.34 |
| A_52_P306387 | NM_178709 | 235315 | D130054N24Rik | 0.574 | 0.504 | 0.652 | 1.816 | 1.515 | 1.965 |
| A_52_P307801 | NM_007847 | 13222 | Defcr-rs2 | 2.149 | 1.602 | 1.28 | 2.942 | 3.1 | 2.825 |
| A_52_P309718 | AK173099; | 218850 | D14Abb1e | 1.006 | 0.813 | 1.127 | 1.186 | 1.29 | 1.289 |
| A_52_P317273 | NM_172846 | 240960 | A230079K17Rik | 1.135 | 1.343 | 0.878 | 1.131 | 1.103 | 1.019 |
| A_52_P320170 | NM_144787 | 76804 | Jmjd2c | 0.502 | 0.764 | 0.8 | 0.999 | 0.89 | 1.597 |
| A_52_P322927 | XM_487182 | 435271 | LOC435271 | 0.444 | 0.68 | 0.767 | 0.515 | 0.426 | 0.637 |
| A_52_P324934 | AK045391; | 16403 | | 0.758 | 0.573 | 0.774 | 1.808 | 1.513 | 1.534 |
| A_52_P32502 | AK020822; | 77944 | A930007B11Rik | 1.212 | 1.019 | 1.21 | 2.052 | 1.291 | 1.824 |
| A_52_P3304 | NM_080446 | 117599 | Helb | 1.004 | 1.239 | 1.059 | 1.614 | 1.432 | 1.418 |
| A_52_P335089 | BE198552; | | | 0.338 | 0.471 | 0.43 | 0.921 | 0.981 | 0.901 |
| A_52_P336259 | AK087367; XM_485576 | 319467 | A930002C04Rik | 0.552 | 0.507 | 0.623 | 1.053 | 0.913 | 0.8 |
| A_52_P344424 | NM_010720 | 16891 | Lipg | 1.688 | 1.328 | 2.214 | 1.439 | 1.09 | 1.069 |
| A_52_P345626 | AY149175; | 386626 | Mzf6d | 0.699 | 0.636 | 0.76 | 1.173 | 1.052 | 1.267 |
| A_52_P34823 | NM_010747 | 17096 | Lyn | 0.523 | 0.589 | 0.625 | 0.371 | 0.427 | 0.434 |
| A_52_P350477 | NM_139295 | 193813 | Mcfd2 | 1.118 | 1.377 | 1.329 | 0.667 | 0.758 | 0.786 |
| A_52_P354363 | NM_026574 | 68142 | | 0.545 | 0.546 | 0.579 | 1.246 | 1.113 | 1.072 |
| A_52_P354785 | AK037929; | 14852 | Gspt1 | 0.487 | 0.598 | 0.585 | 1.587 | 1.447 | 1.483 |
| A_52_P355276 | NM_001002764 | 103677 | AI317223 | 0.704 | 0.605 | 0.784 | 1.531 | 1.432 | 1.606 |
| A_52_P358951 | AK081999; XM_134736 | 271457; 22688 | Rab5a | 1.346 | 1.445 | 1.326 | 0.843 | 0.883 | 0.787 |
| A_52_P359010 | AK129478; | 72193 | Sfrs2ip | 0.54 | 0.706 | 0.574 | 1.484 | 1.398 | 1.403 |
| A_52_P361534 | NM_009521 | 22415 | Wnt3 | 1.193 | 0.891 | 0.969 | 2.247 | 2.459 | 2.231 |
| A_52_P362805 | AK044730; | 269254 | Als4 | 0.692 | 0.79 | 0.89 | 1.216 | 1.11 | 1.17 |
| A_52_P364021 | NM_026713 | 68393 | Mogat1 | 1.295 | 1.109 | 1.272 | 1.124 | 1.301 | 1.292 |
| A_52_P367294 | NM_176966 | 319636 | Ccdc10 | 0.64 | 0.752 | 1.224 | 1.255 | 1.037 | 1.385 |
| A_52_P3698 | NM_001015099 | 217558 | | 0.658 | 0.653 | 0.676 | 1.487 | 1.564 | 1.537 |
| A_52_P370162 | NM_175271 | 78134 | Gpr23 | 1.104 | 1.003 | 1.165 | 1.307 | 1.348 | 1.868 |
| A_52_P376169 | NM_177139 | 320343 | E130115E03Rik | 1.184 | 1.025 | 1.076 | 1.949 | 1.81 | 1.828 |
| A_52_P376502 | BC025011; | 227541 | Camk1d | 0.664 | 0.71 | 0.737 | 0.973 | 0.893 | 1.008 |
| A_52_P377299 | AK048360; | 271377 | Zbtb11 | 0.577 | 0.582 | 0.645 | 1.152 | 1.092 | 1.023 |
| A_52_P380263 | NM_013723 | 27205 | Podxl | 1.179 | 1.159 | 1.66 | 0.899 | 0.909 | 0.942 |
| A_52_P382565 | XM_483943 | 432493 | LOC432493 | 0.228 | 0.273 | 0.254 | 0.607 | 0.622 | 0.462 |
| A_52_P384392 | AK080827; | 328087 | D930001B02 | 0.533 | 0.516 | 0.471 | 1.258 | 1.062 | 1.099 |
| A_52_P384718 | BC052056; XM_283264 | 320873 | | 1.488 | 1.451 | 1.435 | 1.051 | 1.032 | 1.026 |
| A_52_P386544 | NM_026594 | 68172 | 4930517K11Rik | 0.74 | 0.606 | 0.729 | 1.188 | 1.159 | 1.351 |
| A_52_P386682 | AK033609; | 69587 | Rnf3 | 1.349 | 2.019 | 1.908 | 0.901 | 0.891 | 1.063 |
| A_52_P389874 | NM_011918 | 24131 | Ldb3 | 0.702 | 0.892 | 0.937 | 1.166 | 1.063 | 1.078 |
| A_52_P391505 | NM_144513 | 17263 | Gtl2 | 0.0793 | 0.114 | 0.0867 | 0.158 | 0.15 | 0.148 |
| A_52_P399095 | NM_177793 | 327747 | 9030224M15Rik | 1.11 | 0.976 | 1.056 | 1.583 | 1.692 | 1.596 |
| A_52_P400425 | NM_178656 | 193003 | A530088H08Rik | 0.241 | 0.469 | 0.547 | 0.79 | 0.835 | 1.024 |
| A_52_P402786 | NM_008935 | 19126 | Prom1 | 1.498 | 1.71 | 1.749 | 1.028 | 1.023 | 0.92 |
| A_52_P405340 | U76762; | 14158 | Fert2 | 0.434 | 0.372 | 0.598 | 1.654 | 1.494 | 1.493 |
| A_52_P410609 | NM_133685 | 106572 | 1700093E07Rik | 0.956 | 1.237 | 1.105 | 0.415 | 0.492 | 0.422 |
| A_52_P412167 | NM_080467 | 140494 | Atp6v0a4 | 0.553 | 1.045 | 0.878 | 1.777 | 2.017 | 2.418 |
| A_52_P413348 | XM_127674 | 75698 | 3110001K24Rik | 0.593 | 0.709 | 0.685 | 1.434 | 1.352 | 1.356 |
| A_52_P413661 | BC079861; XM_484162 | 217716 | Mlh3 | 0.513 | 0.589 | 0.64 | 1.73 | 1.608 | 1.611 |
| A_52_P416123 | AK020480; | 77358 | 9430069I07Rik | 0.584 | 0.644 | 1.001 | 1.681 | 1.367 | 1.728 |
| A_52_P420369 | NM_029444 | 75801 | 4930447C04Rik | 1.087 | 1.084 | 1.129 | 1.176 | 0.989 | 1.041 |
| A_52_P420608 | NM_201366 | 381371 | Gm1631 | 1.252 | 0.926 | 0.759 | 1.014 | 1.175 | 1.159 |
| A_52_P421234 | NM_133832 | 98711 | Rdh10 | 1.706 | 1.519 | 1.676 | 0.845 | 0.856 | 0.776 |
| A_52_P423384 | BC057551; XM_354546 | 104248 | Cabin1 | 0.481 | 0.424 | 0.586 | 1.189 | 1.05 | 1.408 |
| A_52_P425734 | NM_145146 | 280662 | Afm | 1.466 | 1.63 | 2.105 | 1.902 | 1.789 | 2.093 |
| A_52_P430927 | AK042385; | 11908 | Atf1 | 0.409 | 0.436 | 0.428 | 1.263 | 1.186 | 1.26 |
| A_52_P432464 | AK053863; | 192196 | Luc7l2 | 0.7 | 0.863 | 1.032 | 1.457 | 1.269 | 1.582 |
| A_52_P434549 | NM_023190 | 56215 | Acin1 | 0.489 | 0.399 | 0.44 | 1.376 | 1.207 | 1.143 |
| A_52_P438036 | NM_175271 | 78134 | Gpr23 | 1.082 | 0.96 | 0.968 | 1.634 | 1.593 | 1.618 |
| A_52_P442567 | NM_009099 | 20128 | Trim30 | 1.331 | 1.1 | 1.212 | 1.814 | 1.462 | 1.507 |
| A_52_P445799 | AK046777; | 52490 | D18Ertd289e | 2.088 | 2.22 | 2.899 | 2.952 | 2.963 | 3.531 |
| A_52_P452689 | NM_007498 | 11910 | Atf3 | 0.672 | 0.61 | 0.659 | 0.89 | 0.955 | 0.943 |
| A_52_P458916 | AK016751; | 218850 | D14Abb1e | 0.488 | 0.694 | 0.807 | 1.15 | 1.185 | 1.257 |
| A_52_P460882 | XM_130319; XM_130308 | 73668; 14725 | Ttc21b | 1.498 | 1.698 | 2.077 | 1.06 | 1.276 | 1.219 |
| A_52_P469789 | NM_007588 | 12311 | Calcr | 2.087 | 2.206 | 2.278 | 1.646 | 1.638 | 1.533 |
| A_52_P470663 | | | | 2.339 | 2.72 | 2.405 | 1.069 | 1.105 | 1.018 |
| A_52_P474242 | BE457768; | | | 1.31 | 1.785 | 1.55 | 1.137 | 1.132 | 0.997 |
| A_52_P483885 | AK078465; | 76916 | 4930455C21Rik | 0.763 | 0.712 | 1.027 | 1.428 | 1.429 | 1.483 |
| A_52_P485939 | NM_026228 | 67547 | Slc39a8 | 3.203 | 2.606 | 3.687 | 2.136 | 1.903 | 2.146 |
| A_52_P486234 | AK033497; | 22680 | | 0.658 | 0.943 | 0.756 | 2.133 | 2.185 | 1.908 |
| A_52_P487362 | NM_028980 | 74521 | 8430415E04Rik | 3.136 | 2.67 | 3.124 | 4.295 | 4.001 | 3.546 |
| A_52_P490263 | NM_013819 | 14991 | H2-M3 | 1.272 | 1.518 | 1.745 | 0.961 | 1.184 | 1.166 |
| A_52_P492189 | | | | 2.909 | 3.045 | 3.596 | 2.009 | 2.077 | 2.009 |
| A_52_P500979 | NM_013628 | 18548 | Pcsk1 | 4.338 | 3.184 | 4.164 | 4.966 | 5.086 | 4.923 |
| A_52_P501767 | AK012272; | 20637 | Snrp70 | 0.263 | 0.243 | 0.263 | 1.047 | 1 | 0.926 |
| A_52_P50496 | AY048587; XM_619453 | 114640 | Tifp39 | 1.196 | 1.551 | 1.504 | 1.114 | 1.171 | 1.214 |
| A_52_P505907 | NM_146112 | 227331 | Tnrc15 | 0.219 | 0.415 | 0.633 | 1.334 | 1.307 | 1.752 |
| A_52_P518352 | AK046930; | | | 0.266 | 0.346 | 0.376 | 1.223 | 1.034 | 1.313 |
| A_52_P519870 | AB010340; | | | 0.758 | 0.675 | 1.128 | 0.989 | 0.863 | 1.393 |
| A_52_P523399 | NM_029444 | 75801 | 4930447C04Rik | 1.167 | 0.873 | 1.364 | 1.373 | 1.087 | 1.771 |
| A_52_P532323 | NM_178410 | 218518 | Marveld2 | 1.824 | 2.102 | 1.904 | 0.871 | 0.884 | 1.027 |
| A_52_P532910 | NM_024427 | 22003 | Tpm1 | 1.081 | 1.106 | 1.238 | 0.891 | 0.87 | 0.851 |
| A_52_P537140 | AK047235; | | | 0.566 | 0.565 | 0.531 | 1.029 | 1.127 | 1.156 |
| A_52_P538515 | BC049181; | 226412 | R3hdm | 0.209 | 0.659 | 0.565 | 1.913 | 1.725 | 1 |
| A_52_P549815 | XM_621314 | 109620 | | 0.982 | 1.23 | 0.97 | 0.813 | 0.899 | 0.906 |
| A_52_P553100 | AK040413; | | | 0.696 | 0.886 | 0.727 | 2.086 | 2.022 | 1.86 |
| A_52_P553386 | AK041018; | | | 0.235 | 0.571 | 0.685 | 1.632 | 1.95 | 1.925 |
| A_52_P555603 | AK032770; | 11787 | Apbb2 | 0.273 | 0.355 | 0.304 | 1.021 | 1.021 | 1.054 |
| A_52_P556014 | AK087367; XM_620168 | 545738 | A930002C04Rik | NO DATA | 0.365 | 0.504 | 0.806 | 0.777 | 0.943 |
| A_52_P557293 | NM_015775 | 50528 | Tmprss2 | 1.142 | 1.091 | 1.111 | 1.208 | 1.127 | 1.225 |
| A_52_P557940 | AK004302; | 66704 | 4921506I22Rik | 0.544 | 0.595 | 0.569 | 1.371 | 1.379 | 1.241 |
| A_52_P560006 | NM_008426 | 16519 | Kcnj3 | 2.321 | 2.818 | 2.608 | 2.191 | 1.875 | 2.19 |
| A_52_P561046 | AK044509; | | | 0.146 | 0.211 | 0.311 | 0.792 | 0.777 | 1.011 |
| A_52_P565940 | AF419220; | 18193 | Nsd1 | 0.487 | 0.593 | 0.616 | 1.236 | 1.181 | 1.08 |
| A_52_P566681 | NM_153581 | 234267 | Gpm6a | 1.117 | 1.337 | 1.114 | 1.045 | 1.073 | 0.961 |
| A_52_P569375 | NM_010203 | 14176 | Fgf5 | 4.338 | 3.206 | 3.958 | 5.889 | 5.927 | 6.75 |
| A_52_P57078 | AK052455; | | | 0.552 | 0.439 | 0.686 | 1.676 | 1.452 | 1.294 |
| A_52_P571746 | XM_355533; XM_357399 | 230863 | LOC381571 | 1.076 | 1.21 | 1.362 | 2.199 | 2.23 | 2.231 |
| A_52_P571987 | AK053601; | 15525 | | 0.285 | 0.758 | 0.643 | 1.776 | 0.839 | 1.405 |
| A_52_P574668 | NM_011851 | 23959 | Nt5e | 1.611 | 1.127 | 1.284 | 1.764 | 1.707 | 1.826 |
| A_52_P575505 | NM_008994 | 19302 | Pxmp3 | 1.123 | 1.07 | 1.092 | 0.797 | 0.815 | 0.802 |
| A_52_P577072 | NM_011901 | 24074 | Taf7 | 0.654 | 0.591 | 0.759 | 1.655 | 1.407 | 1.87 |
| A_52_P581100 | NM_027285 | 70005 | 1700029I01Rik | 1.164 | 1.1 | 1.141 | 1.525 | 1.383 | 1.305 |
| A_52_P581138 | NM_212450 | 329506 | D2Ertd485e | 0.457 | 0.451 | 0.472 | 1.47 | 1.244 | 1.239 |
| A_52_P584874 | NM_152895 | 75605 | Jarid1b | 0.737 | 0.83 | 0.752 | 1.242 | 1.251 | 1.242 |
| A_52_P586157 | NM_022309 | 12400 | Cbfb | 1.417 | 1.551 | 1.338 | 0.773 | 0.863 | 0.903 |
| A_52_P59318 | NM_133832 | 98711 | Rdh10 | 1.689 | 1.611 | 1.584 | 0.876 | 1.002 | 0.955 |
| A_52_P601333 | AK078961; | 67786 | 6230424C14Rik | 0.336 | 0.445 | 0.476 | 1.075 | 1.112 | 0.946 |
| A_52_P620345 | NM_133979 | 102566 | AI604832 | 1.26 | 1.488 | 1.375 | 0.778 | 0.752 | 0.729 |
| A_52_P626069 | AK040994; XM_284439 | 109151 | Chd9 | 0.449 | 0.512 | 0.687 | 1.302 | 1.102 | 1.65 |
| A_52_P628212 | AK044339; | 26380 | | 1.058 | 1.337 | 0.967 | 4.002 | 3.276 | 3.435 |
| A_52_P630964 | NM_027533 | 70747 | Tspan2 | 1.217 | 1.314 | 1.26 | 0.815 | 0.863 | 0.897 |
| A_52_P634829 | NM_023835 | 76681 | Trim12 | 1.52 | 1.676 | 1.775 | 1.472 | 1.69 | 1.577 |
| A_52_P636199 | NM_008017 | 14211 | Smc2l1 | 0.482 | 0.452 | 0.534 | 1.172 | 1.125 | 1.415 |
| A_52_P636559 | AK053008; | 268469 | 9530033F24Rik | 0.506 | 0.53 | 0.567 | 1.485 | 1.451 | 1.363 |
| A_52_P650215 | NM_029278 | 75416 | 2610033H07Rik | 0.869 | 0.561 | 1.153 | 1.294 | 1.106 | 1.919 |
| A_52_P651469 | NM_001012726 | 503692 | | 1.087 | 2.041 | 1.585 | 2.114 | 1.954 | 2.198 |
| A_52_P654624 | AK005333; | 236604 | 4933439C20Rik | 0.543 | 0.696 | 0.523 | 1.316 | 1.231 | 1.139 |
| A_52_P655008 | AK044382; | 320678 | A930037G23Rik | 0.364 | 0.363 | 0.425 | 1.339 | 1.302 | 1.272 |
| A_52_P656336 | NM_023733 | 74114 | Crot | 1.233 | 1.547 | 1.442 | 0.983 | 1.067 | 0.998 |
| A_52_P661144 | XM_485365 | | | 0.511 | 0.546 | 0.638 | 1.627 | 1.676 | 2.009 |
| A_52_P6621 | | | | 0.661 | 0.506 | 1.607 | 1.455 | 1.079 | 2.626 |
| A_52_P672761 | NM_013689 | 21682 | Tec | 0.969 | 1.307 | 1.208 | 0.717 | 0.737 | 0.713 |
| A_52_P676403 | NM_019494 | 56066 | Cxcl11 | 3.86 | 3.177 | 3.663 | 2.834 | 2.723 | 2.826 |
| A_52_P676510 | NM_011579 | 21822 | Tgtp | 1.166 | 1.03 | 1.064 | 1.15 | 1.119 | 1.082 |
| A_52_P680436 | NM_025793 | 66840 | Wdr45l | 1.338 | 1.213 | 1.346 | 0.755 | 0.702 | 0.805 |
| A_52_P680941 | NM_010284 | 14600 | Ghr | 1.72 | 1.563 | 1.682 | 2.058 | 1.978 | 2.065 |
| A_52_P68702 | NM_145148 | 232288 | Frmd4b | 0.928 | 1.071 | 0.877 | 1.242 | 1.234 | 1.139 |
| A_52_P68942 | NM_026952 | 69131 | Crk7 | 0.535 | 0.706 | 0.746 | 1.388 | 1 | 1.146 |
| A_52_P731549 | AK085965; | 67939 | 2010316F05Rik | 0.236 | 0.333 | 0.323 | 1.081 | 1 | 1.122 |
| A_52_P74441 | NM_011570 | 21753 | Tes | 1.498 | 1.167 | 1.425 | 0.853 | 0.882 | 0.885 |
| A_52_P75971 | NM_026599 | 68178 | Cgnl1 | 0.432 | 0.439 | 0.529 | 0.801 | 0.761 | 0.771 |
| A_52_P78168 | AK031518; | | | 0.277 | 0.385 | 0.299 | 0.704 | 0.652 | 0.544 |
| A_52_P795628 | AK086352; | | | 0.551 | 1.049 | 0.818 | 3.259 | 4.511 | 4.2 |
| A_52_P804224 | | | | 0.299 | 0.314 | 0.291 | 1.263 | 1.2 | 1.068 |
| A_52_P812362 | BC016205; | 408064 | BC064078 | 1.424 | 1.508 | 1.444 | 1.323 | 1.468 | 1.262 |
| A_52_P81252 | NM_172599 | 218978 | D14Ertd436e | 0.265 | 0.408 | 0.488 | 1.153 | 1.046 | 0.927 |
| A_52_P819356 | AK041684; | | | 0.651 | 0.704 | 0.763 | 1.654 | 1.657 | 1.821 |
| A_52_P82488 | NM_021476 | 58861 | Cysltr1 | 1.919 | 1.474 | 1.811 | 2.106 | 2.164 | 2.621 |
| A_52_P826666 | AK007019; | 51799 | Rap2ip | 0.894 | 1.131 | 1.14 | 2.913 | 2.479 | 3.106 |
| A_52_P82722 | NM_009988 | 13052 | Cxadr | 0.584 | 0.807 | 0.998 | 0.725 | 0.811 | 0.798 |
| A_52_P827911 | AK081941; | | | 0.209 | 0.476 | 0.487 | 1.73 | 1.566 | 1.778 |
| A_52_P84015 | AK037762; | 11863 | | 0.695 | 0.924 | 0.842 | 2.965 | 3.344 | 3.023 |
| A_52_P85805 | NM_009525 | 22419 | Wnt5b | 1.366 | 1.006 | 1.074 | 0.994 | 0.884 | 1.009 |
| A_52_P87839 | NM_013657 | 20348 | Sema3c | 1.257 | 0.996 | 1.513 | 0.958 | 1.004 | 0.817 |
| A_52_P93837 | NM_008604 | 17380 | Mme | 1.516 | 1.805 | 1.761 | 1.442 | 1.369 | 1.329 |
| A_52_P947879 | AK048751; | | | 0.354 | 0.496 | 0.449 | 1.501 | 1.634 | 1.919 |
| A_52_P957500 | XM_622354 | | | 0.371 | 0.532 | 0.397 | 1.528 | 1.372 | 1.305 |
| A_52_P995717 | AK045222; | | | 0.672 | 0.74 | 0.709 | 2.392 | 1.854 | 2.412 |

TABLE 2A
292 Gene Set (Germline Comeptent)

| | | | | Clone 1 | | | Clone 2 | | |
|---|---|---|---|---|---|---|---|---|---|
| Agilent Probe ID | Genbank | Entrez ID | Gene Symbol | rep. 1 | rep. 2 | rep. 3 | rep. 1 | rep. 2 | rep. 3 |
| A_51_P237040 | NM_008711 | 18121 | Nog | 0.266 | 0.299 | 0.281 | 0.396 | 0.368 | 0.407 |
| A_51_P335460 | NM_009132 | 20259 | Scin | 0.718 | 0.631 | 0.719 | 0.678 | 0.674 | 0.666 |
| A_52_P391505 | NM_144513 | 17263 | Gtl2 | 1.36 | 1.291 | 1.327 | 1.053 | 1.04 | 0.852 |
| A_51_P497100 | NM_010706 | 16855 | Lgals4 | 2.115 | 2.049 | 2.368 | 1.716 | 1.861 | 1.527 |
| A_51_P378371 | | | | 0.454 | 0.542 | 0.39 | 0.468 | 0.484 | 0.468 |
| A_51_P298266 | NM_025681 | 66643 | Lix1 | 0.507 | 0.414 | 0.416 | 0.554 | 0.584 | 0.54 |
| A_52_P269158 | NM_001003948 | 98496 | AL024069 | 0.316 | 0.33 | 0.33 | 0.388 | 0.393 | 0.443 |
| A_51_P311038 | BC009666; XM_134720 | 57267 | Apba3 | 0.334 | 0.341 | 0.339 | 0.446 | 0.472 | 0.482 |
| A_51_P457187 | NM_028075 | 72049 | Tnfrsf13c | 2.82 | 3.275 | 3.026 | 2.654 | 3.432 | 2.546 |
| A_52_P68702 | NM_145148 | 232288 | Frmd4b | 0.545 | 0.476 | 0.538 | 0.501 | 0.43 | 0.424 |
| A_52_P293443 | AK087208; | 13819 | | 1.278 | 1.075 | 1.13 | 1.496 | 1.604 | 1.349 |
| A_51_P157986 | NM_029930 | 77574 | 3321401G04Rik | 0.426 | 0.339 | 0.345 | 0.368 | 0.318 | 0.37 |
| A_52_P1013441 | | | | 3.248 | 3.164 | 3.191 | 4.558 | 5.61 | 4.683 |
| A_51_P418375 | NM_023844 | 67374 | Jam2 | 1.691 | 1.467 | 1.625 | 2.142 | 2.132 | 2.288 |
| A_52_P557940 | AK004302; | 66704 | 4921506I22Rik | 2.114 | 1.863 | 2.221 | 2.17 | 2.013 | 2.051 |
| A_51_P480311 | NM_010168 | 14061 | F2 | 1.991 | 1.809 | 1.969 | 2.465 | 2.4 | 1.874 |
| A_52_P200458 | NM_010242 | 14345 | Fut4 | 0.429 | 0.343 | 0.355 | 0.453 | 0.391 | 0.512 |
| A_51_P463846 | NM_145545 | 229900 | 9830147J24Rik | 0.694 | 0.519 | 0.59 | 0.86 | 0.816 | 0.817 |
| A_51_P162474 | AF285577; | 107815 | Scml2 | 0.539 | 0.413 | 0.405 | 0.643 | 0.645 | 0.71 |
| A_51_P165244 | NM_018734 | 55932 | Gbp4 | 0.585 | 0.512 | 0.474 | 1.008 | 0.981 | 1.006 |
| A_52_P59318 | NM_133832 | 98711 | Rdh10 | 0.306 | 0.273 | 0.275 | 0.373 | 0.395 | 0.445 |
| A_52_P438036 | NM_175271 | 78134 | Gpr23 | 0.376 | 0.334 | 0.36 | 0.455 | 0.441 | 0.489 |
| A_52_P474242 | BE457768; | | | 0.499 | 0.53 | 0.46 | 0.69 | 0.658 | 0.726 |
| A_51_P499854 | NM_010284 | 14600 | Ghr | 0.407 | 0.375 | 0.412 | 0.413 | 0.429 | 0.489 |
| A_51_P455647 | NM_009801 | 12349 | Car2 | 0.335 | 0.288 | 0.301 | 0.568 | 0.516 | 0.638 |
| A_52_P103929 | NM_009306 | 20979 | Syt1 | 0.382 | 0.356 | 0.39 | 0.419 | 0.498 | 0.423 |
| A_51_P149469 | NM_009608 | 11464 | Actc1 | 0.324 | 0.387 | 0.384 | 0.496 | 0.551 | 0.471 |
| A_52_P557293 | NM_015775 | 50528 | Tmprss2 | 0.277 | 0.205 | 0.179 | 0.45 | 0.457 | 0.355 |
| A_52_P50496 | AY048587; XM_619453 | 114640 | Tifp39 | 0.454 | 0.501 | 0.422 | 0.661 | 0.638 | 0.694 |
| A_51_P180413 | NM_176930 | 319504 | C130076O07Rik | 0.359 | 0.393 | 0.415 | 0.471 | 0.514 | 0.494 |
| A_51_P229613 | NM_008792 | 18549 | Pcsk2 | 0.512 | 0.483 | 0.515 | 0.58 | 0.58 | 0.504 |
| A_51_P121635 | NM_030700 | 80884 | Maged2 | 0.378 | 0.371 | 0.409 | 0.492 | 0.475 | 0.481 |
| A_51_P481777 | NM_018756 | 54382 | Tcstv1 | 1.532 | 1.822 | 1.792 | 2.619 | 3.155 | 2.956 |
| A_51_P412817 | NM_177617 | 216851 | D330014H01Rik | 3.151 | 3.039 | 3.326 | 2.136 | 2.194 | 1.865 |
| A_51_P387528 | AK019664; | 78801 | Ak7 | 2.693 | 2.814 | 2.57 | 3.786 | 3.834 | 3.71 |
| A_52_P78168 | AK031518; | | | 2.108 | 1.677 | 1.617 | 1.783 | 1.791 | 1.711 |
| A_52_P274960 | NM_001005508 | 226652 | 6030405P05Rik | 1.83 | 1.82 | 1.948 | 2.07 | 2.181 | 2.076 |
| A_52_P1188270 | AK042362; | | | 3.076 | 1.828 | 1.917 | 2.932 | 3.046 | 2.87 |
| A_51_P291227 | NM_001005508 | 226652 | 6030405P05Rik | 1.876 | 1.826 | 1.86 | 2.054 | 2.185 | 2.004 |
| A_52_P377299 | AK048360; | 271377 | Zbtb11 | 1.927 | 1.892 | 1.923 | 1.678 | 1.79 | 1.546 |
| A_51_P297131 | NM_009502 | 22330 | Vcl | 0.402 | 0.372 | 0.378 | 0.411 | 0.377 | 0.401 |
| A_52_P804224 | | | | 1.522 | 1.688 | 1.828 | 2.31 | 2.487 | 2.241 |
| A_51_P442894 | AK048310; | 621603 | C130048D07Rik | 2.632 | 2.364 | 2.281 | 3.657 | 3.714 | 3.102 |
| A_52_P309718 | AK173099; | 218850 | D14Abb1e | 2.324 | 2.579 | 2.18 | 2.614 | 2.72 | 2.711 |
| A_52_P359010 | AK129478; | 72193 | Sfrs2ip | 2.152 | 2.149 | 2.122 | 2.349 | 2.38 | 2.318 |
| A_51_P410813 | AK079732; | | | 2.732 | 2.716 | 2.559 | 2.898 | 2.605 | 2.3 |
| A_52_P382565 | XM_483943 | 432493 | LOC432493 | 0.976 | 1.222 | 1.221 | 1.492 | 1.439 | 1.198 |
| A_51_P333253 | NM_178440 | 246177 | Myo1g | 1.419 | 1.413 | 1.621 | 1.544 | 1.504 | 1.394 |
| A_52_P113190 | NM_053214 | 17916 | Myo1f | 1.428 | 1.513 | 1.396 | 1.988 | 1.959 | 2.083 |
| A_51_P473953 | XM_358600; XM_355454 | 381498 | 4930592C13Rik | 0.42 | 0.306 | 0.298 | 0.365 | 0.365 | 0.38 |
| A_52_P519870 | AB010340; | | | 2.391 | 3.34 | 2.621 | 3.76 | 3.72 | 1.955 |
| A_51_P266403 | AK009163; | 76730 | 2310005C01Rik | 3.079 | 3.053 | 3.092 | 2.733 | 2.967 | 2.734 |
| A_52_P3698 | NM_001015099 | 217558 | | 1.885 | 1.84 | 1.762 | 2.392 | 2.296 | 2.486 |
| A_52_P581100 | NM_027285 | 70005 | 1700029I01Rik | 2.295 | 3.266 | 2.708 | 3.168 | 3.146 | 2.879 |
| A_51_P276305 | NM_009292 | 20899 | Stra8 | 3.137 | 3.069 | 3.174 | 2.824 | 2.972 | 2.777 |
| A_51_P455997 | Y13832; | 17263 | Gtl2 | 1.505 | 1.455 | 1.44 | 0.998 | 1.082 | 0.998 |
| A_52_P656336 | NM_023733 | 74114 | Crot | 0.407 | 0.351 | 0.381 | 0.755 | 0.647 | 0.8 |
| A_51_P512172 | AK010953; XM_125673 | 52463 | Cxxc6 | 2.864 | 2.384 | 2.216 | 2.184 | 2.11 | 2.068 |
| A_51_P282227 | NM_020577 | 57344 | As3mt | 2.651 | 2.607 | 2.876 | 3.224 | 3.327 | 3.147 |
| A_52_P386544 | NM_026594 | 68172 | 4930517K11Rik | 2.843 | 2.618 | 2.802 | 2.673 | 2.913 | 2.894 |
| A_51_P125679 | AK010725; XM_134948 | 73673 | 2410076I21Rik | 2.867 | 2.332 | 2.464 | 3.518 | 3.661 | 3.727 |
| A_51_P164504 | NM_007469 | 11812 | Apoc1 | 3.245 | 2.908 | 3.052 | 2.863 | 3.059 | 2.591 |
| A_52_P240706 | NM_007543 | 26367 | Ceacam2 | 2.285 | 1.866 | 1.892 | 2.654 | 2.556 | 2.628 |
| A_52_P420369 | NM_029444 | 75801 | 4930447C04Rik | 3.256 | 2.433 | 2.971 | 2.042 | 2.189 | 2.206 |
| A_51_P242399 | NM_031170 | 16691 | Krt2-8 | 0.193 | 0.233 | 0.208 | 0.403 | 0.4 | 0.39 |
| A_51_P158037 | NM_013505 | 13506 | Dsc2 | 0.271 | 0.249 | 0.258 | 0.437 | 0.415 | 0.453 |
| A_52_P258116 | NM_009521 | 22415 | Wnt3 | 0.156 | 0.164 | 0.16 | 0.312 | 0.309 | 0.296 |
| A_52_P286166 | NM_001002927 | 18619 | Penk1 | 0.497 | 0.324 | 0.339 | 0.671 | 0.658 | 0.647 |
| A_52_P399095 | NM_177793 | 327747 | 9030224M15Rik | 0.366 | 0.325 | 0.163 | 0.433 | 0.401 | 0.466 |
| A_52_P470663 | | | | 0.377 | 0.334 | 0.403 | 0.467 | 0.439 | 0.566 |
| A_52_P376169 | NM_177139 | 320343 | E130115E03Rik | 0.314 | 0.285 | 0.295 | 0.539 | 0.508 | 0.528 |
| A_51_P512085 | NM_130449 | 140792 | Colec12 | 0.335 | 0.364 | 0.296 | 0.382 | 0.368 | 0.385 |
| A_52_P676403 | NM_019494 | 56066 | Cxcl11 | 0.525 | 0.596 | 0.597 | 0.761 | 0.809 | 0.704 |
| A_51_P354307 | NM_172469 | 209195 | Clic6 | 0.43 | 0.319 | 0.329 | 0.398 | 0.398 | 0.387 |
| A_52_P569375 | NM_010203 | 14176 | Fgf5 | 0.776 | 0.678 | 0.554 | 0.45 | 0.467 | 0.574 |
| A_51_P162481 | NM_133194 | 107815 | Scml2 | 0.432 | 0.357 | 0.385 | 0.594 | 0.55 | 0.618 |
| A_51_P202911 | NM_144882 | 67198 | 2810022L02Rik | 0.297 | 0.283 | 0.328 | 0.346 | 0.333 | 0.365 |
| A_51_P418029 | NM_175541 | 245631 | Mum1l1 | 0.319 | 0.265 | 0.278 | 0.419 | 0.397 | 0.534 |
| A_52_P93837 | NM_008604 | 17380 | Mme | 0.559 | 0.439 | 0.513 | 0.653 | 0.573 | 0.747 |
| A_51_P275435 | NM_172411 | 71874 | 2310007B03Rik | 0.368 | 0.492 | 0.206 | 0.875 | 0.83 | 0.857 |
| A_51_P199888 | NM_026082 | 67299 | Dock7 | 0.407 | 0.403 | 0.245 | 0.558 | 0.428 | 0.546 |
| A_52_P211956 | NM_025658 | 66607 | Ms4a4d | 0.61 | 0.455 | 0.457 | 0.534 | 0.519 | 0.685 |
| A_51_P485985 | NM_134163 | 171170 | Mbnl3 | 0.223 | 0.181 | 0.161 | 0.625 | 0.543 | 0.697 |
| A_52_P630964 | NM_027533 | 70747 | Tspan2 | 0.361 | 0.283 | 0.324 | 0.499 | 0.473 | 0.525 |
| A_51_P294505 | NM_025658 | 66607 | Ms4a4d | 0.63 | 0.414 | 0.365 | 0.532 | 0.521 | 0.629 |
| A_52_P82488 | NM_021476 | 58861 | Cysltr1 | 0.319 | 0.379 | 0.306 | 0.5 | 0.438 | 0.534 |
| A_52_P634829 | NM_023835 | 76681 | Trim12 | 0.59 | 0.561 | 0.672 | 1.012 | 0.996 | 0.866 |
| A_52_P460882 | XM_130319; XM_130308 | 73668; 14725 | Ttc21b | 0.412 | 0.637 | 0.533 | 0.396 | 0.633 | 0.502 |
| A_52_P197826 | NM_010397 | 15039 | H2-T22 | 0.541 | 0.505 | 0.41 | 0.653 | 0.634 | 0.622 |
| A_51_P240253 | NM_019662 | 56437 | Rrad | 0.316 | 0.322 | 0.314 | 0.392 | 0.337 | 0.342 |
| A_51_P101975 | NM_010205 | 14179 | Fgf8 | 0.412 | 0.39 | 0.413 | 0.415 | 0.454 | 0.385 |
| A_52_P21595 | NM_028238 | 72433 | Rab38 | 0.234 | 0.188 | 0.168 | 0.439 | 0.467 | 0.472 |
| A_52_P402786 | NM_008935 | 19126 | Prom1 | 0.37 | 0.396 | 0.335 | 0.551 | 0.506 | 0.594 |
| A_52_P126158 | NM_008326 | 15944 | Ifi1 | 0.237 | 0.208 | 0.22 | 0.415 | 0.412 | 0.425 |
| A_52_P676510 | NM_011579 | 21822 | Tgtp | 0.225 | 0.162 | 0.155 | 0.382 | 0.378 | 0.362 |
| A_52_P175685 | NM_008390 | 16362 | Irf1 | 0.401 | 0.37 | 0.3 | 0.663 | 0.637 | 0.749 |
| A_52_P384718 | BC052056; XM_283264 | 320873 | | 0.508 | 0.441 | 0.458 | 0.541 | 0.512 | 0.578 |
| A_52_P176333 | NM_001005787 | 12695 | Cipp | 0.372 | 0.357 | 0.365 | 0.452 | 0.392 | 0.42 |
| A_52_P358951 | AK081999; XM_134736 | 271457; 22688 | Rab5a | 0.417 | 0.378 | 0.448 | 0.464 | 0.449 | 0.46 |
| A_51_P168646 | AK039580; | | | 0.442 | 0.434 | 0.509 | 0.537 | 0.548 | 0.598 |
| A_52_P620345 | NM_133979 | 102566 | AI604832 | 0.368 | 0.402 | 0.382 | 0.441 | 0.438 | 0.493 |
| A_52_P253179 | AK077477; | 16009 | Igfbp3 | 0.397 | 0.511 | 0.257 | 0.517 | 0.468 | 0.482 |
| A_51_P397003 | XM_133990 | 68904 | 1110065L07Rik | 0.45 | 0.438 | 0.438 | 0.542 | 0.479 | 0.613 |
| A_52_P74441 | NM_011570 | 21753 | Tes | 0.423 | 0.418 | 0.328 | 0.54 | 0.558 | 0.659 |
| A_52_P255250 | NM_009543 | 22644 | Rnf103 | 0.532 | 0.371 | 0.413 | 0.471 | 0.455 | 0.491 |
| A_52_P575505 | NM_008994 | 19302 | Pxmp3 | 0.305 | 0.306 | 0.317 | 0.463 | 0.445 | 0.525 |
| A_52_P469789 | NM_007588 | 12311 | Calcr | 0.48 | 0.383 | 0.445 | 0.625 | 0.707 | 0.696 |
| A_51_P345422 | NM_010747 | 17096 | Lyn | 0.244 | 0.247 | 0.256 | 0.373 | 0.392 | 0.424 |
| A_51_P356003 | NM_001007580 | 333564 | Gm784 | 0.494 | 0.445 | 0.513 | 0.509 | 0.483 | 0.534 |
| A_52_P680941 | NM_010284 | 14600 | Ghr | 0.389 | 0.396 | 0.371 | 0.448 | 0.445 | 0.488 |
| A_52_P380263 | NM_013723 | 27205 | Podxl | 0.395 | 0.422 | 0.286 | 0.532 | 0.511 | 0.504 |
| A_52_P286528 | BC055771; | 102954 | Nudt11 | 0.411 | 0.273 | 0.263 | 0.433 | 0.371 | 0.517 |
| A_51_P105068 | NM_027990 | 71897 | 2310010M24Rik | 0.459 | 0.371 | 0.462 | 0.48 | 0.484 | 0.498 |
| A_52_P132608 | NM_172685 | 229731 | Slc25a24 | 0.358 | 0.291 | 0.25 | 0.504 | 0.401 | 0.432 |
| A_51_P467505 | NM_013689 | 21682 | Tec | 0.28 | 0.242 | 0.227 | 0.502 | 0.464 | 0.499 |
| A_52_P153685 | AK129323; | 70422 | 2810417D08Rik | 0.452 | 0.397 | 0.407 | 0.587 | 0.526 | 0.735 |
| A_51_P174943 | NM_008485 | 16782 | Lamc2 | 0.234 | 0.255 | 0.286 | 0.433 | 0.411 | 0.451 |
| A_52_P301821 | NM_053078 | 27528 | D0H4S114 | 0.442 | 0.427 | 0.469 | 0.472 | 0.525 | 0.486 |
| A_52_P532910 | NM_024427 | 22003 | Tpm1 | 0.358 | 0.392 | 0.35 | 0.386 | 0.39 | 0.433 |
| A_51_P521084 | NM_025626 | 66540 | 3110001A13Rik | 0.408 | 0.406 | 0.391 | 0.474 | 0.456 | 0.494 |
| A_52_P680436 | NM_025793 | 66840 | Wdr45l | 0.344 | 0.369 | 0.331 | 0.651 | 0.557 | 0.675 |
| A_51_P164219 | NM_011909 | 24110 | Usp18 | 0.435 | 0.307 | 0.303 | 0.435 | 0.448 | 0.457 |
| A_52_P421234 | NM_133832 | 98711 | Rdh10 | 0.356 | 0.309 | 0.338 | 0.406 | 0.4 | 0.427 |
| A_52_P586157 | NM_022309 | 12400 | Cbfb | 0.481 | 0.354 | 0.384 | 0.606 | 0.523 | 0.75 |
| A_51_P262171 | NM_008326 | 15944 | Ifi1 | 0.29 | 0.264 | 0.261 | 0.474 | 0.475 | 0.507 |
| A_52_P452689 | NM_007498 | 11910 | Atf3 | 0.291 | 0.326 | 0.293 | 0.307 | 0.321 | 0.307 |
| A_52_P295933 | AK045060; | 14051 | B130023L16Rik | 0.336 | 0.293 | 0.594 | 0.466 | 0.5 | 0.466 |
| A_51_P222773 | NM_010446 | 15376 | Foxa2 | 0.423 | 0.314 | 0.4 | 0.447 | 0.809 | 0.489 |
| A_52_P361534 | NM_009521 | 22415 | Wnt3 | 0.0992 | 0.0782 | 0.0774 | 0.211 | 0.198 | 0.243 |
| A_51_P507880 | NM_028980 | 74521 | 8430415E04Rik | 0.223 | 0.221 | 0.235 | 0.35 | 0.326 | 0.332 |
| A_52_P574668 | NM_011851 | 23959 | Nt5e | 0.15 | 0.159 | 0.148 | 0.316 | 0.285 | 0.335 |
| A_52_P957500 | XM_622354 | | | 2.438 | 1.963 | 2.267 | 2.142 | 2.231 | 1.855 |
| A_52_P345626 | AY149175; | 386626 | Mzf6d | 2.677 | 3.059 | 3.002 | 3.203 | 2.748 | 3.757 |
| A_52_P84015 | AK037762; | 11863 | | 3.675 | 3.344 | 3.705 | 4.008 | 3.981 | 3.736 |
| A_52_P432464 | AK053863; | 192196 | Luc7l2 | 2.643 | 3.226 | 2.225 | 1.973 | 2.004 | 1.643 |
| A_52_P376502 | BC025011; | 227541 | Camk1d | 1.575 | 1.641 | 1.563 | 1.989 | 2.543 | 1.959 |
| A_52_P354785 | AK037929; | 14852 | Gspt1 | 2.114 | 1.965 | 1.835 | 1.904 | 1.907 | 2.055 |
| A_51_P398766 | NM_010259 | 14468 | Gbp1 | 0.374 | 0.372 | 0.361 | 0.894 | 0.876 | 0.949 |
| A_52_P486234 | AK033497; | 22680 | | 3.81 | 2.878 | 2.669 | 3.19 | 3.59 | 3.068 |
| A_52_P628212 | AK044339; | 26380 | | 9.353 | 5.604 | 7.112 | 6.273 | 6.769 | 7.237 |
| A_52_P445799 | AK046777; | 52490 | D18Ertd289e | 0.676 | 0.602 | 0.389 | 0.613 | 0.532 | 0.592 |
| A_51_P268167 | BC037232; XM_621365 | 229363; 218850 | Gmps | 2.665 | 2.898 | 2.35 | 2.187 | 2.366 | 2.198 |
| A_51_P153079 | NM_133923 | 101100 | 4833441J24Rik | 1.312 | 1.174 | 1.271 | 1.222 | 1.343 | 1.299 |
| A_52_P584874 | NM_152895 | 75605 | Jarid1b | 2.228 | 2.064 | 2.215 | 1.844 | 1.912 | 1.769 |
| A_52_P307801 | NM_007847 | 13222 | Defcr-rs2 | 0.119 | 0.091 | 0.0705 | 0.133 | 0.175 | 0.108 |
| A_52_P163148 | AB010370; | 544945 | | 2.883 | 2.86 | 2.64 | 3.58 | 3.156 | 3.085 |
| A_51_P171728 | NM_011926 | 26365 | Ceacam1 | 2.703 | 1.992 | 2.013 | 3.11 | 3.457 | 2.977 |
| A_51_P141660 | AK053665; XM_358382 | 67776; 235380 | Loh11cr2a | 2.996 | 3.339 | 3.08 | 2.433 | 2.601 | 2.365 |
| A_52_P532323 | NM_178410 | 218518 | Marveld2 | 0.399 | 0.326 | 0.264 | 0.434 | 0.363 | 0.431 |
| A_51_P337125 | NM_010566 | 16331 | Inpp5d | 3.162 | 2.936 | 3.033 | 4.399 | 4.495 | 4.161 |
| A_52_P336259 | AK087367; XM_485576 | 319467 | A930002C04Rik | 1.736 | 2.368 | 2.023 | 1.677 | 1.679 | 1.401 |
| A_51_P318577 | AK010729; | 73671 | 2410078J06Rik | 3.283 | 4.227 | 3.815 | 6.599 | 6.454 | 5.366 |
| A_52_P324934 | AK045391; | 16403 | | 1.983 | 2.443 | 1.872 | 2.691 | 2.881 | 2.226 |
| A_52_P826666 | AK007019; | 51799 | Rap2ip | 4.063 | 3.142 | 3.547 | 3.365 | 3.764 | 3.267 |
| A_51_P200223 | XM_619497 | | | 2.332 | 2.259 | 2.383 | 1.575 | 1.797 | 1.553 |
| A_51_P228472 | NM_008343 | 16009 | Igfbp3 | 0.288 | 0.22 | 0.276 | 0.283 | 0.322 | 0.364 |
| A_52_P206573 | NM_027427 | 70439 | Taf15 | 2.14 | 3.003 | 2.282 | 1.904 | 2.123 | 1.956 |
| A_52_P354363 | NM_026574 | 68142 | | 1.74 | 1.737 | 1.937 | 1.525 | 1.61 | 1.378 |
| A_52_P651469 | NM_001012726 | 503692 | | 5.11 | 4.303 | 5.085 | 5.606 | 6.363 | 4.012 |
| A_51_P463789 | BC051947; | 58212 | | 3.253 | 2.961 | 3.321 | 2.093 | 2.306 | 1.934 |
| A_52_P18887 | NM_011468 | 20755 | Sprr2a | 0.21 | 0.171 | 0.214 | 0.282 | 0.277 | 0.25 |
| A_51_P223132 | NM_031183; XM_486227 | 83395; 70549 | Sp6 | 2.329 | 2.088 | 2.196 | 1.457 | 1.639 | 1.36 |
| A_52_P223083 | BC038323; | 72096 | 2010208K18Rik | 4.486 | 6.15 | 3.745 | 3.784 | 3.918 | 3.378 |
| A_52_P523399 | NM_029444 | 75801 | 4930447C04Rik | 3.312 | 7.019 | 4.341 | 3.597 | 4.009 | 2.819 |
| A_52_P282000 | AK020248; | 13800 | Enah | 2.7 | 2.435 | 2.683 | 2.632 | 2.812 | 2.59 |
| A_52_P152983 | AK011739; | 26886 | Cenph | 2.134 | 3.107 | 2.171 | 2.114 | 2.408 | 2.327 |
| A_52_P26416 | AF106279; | 16782 | | 0.254 | 0.252 | 0.241 | 0.358 | 0.391 | 0.378 |
| A_51_P102081 | NM_007847 | 13222 | Defcr-rs2 | 0.061 | 0.0426 | 0.0644 | 0.0791 | 0.158 | 0.0864 |
| A_51_P285725 | NM_009309 | 20997 | T | 0.141 | 0.102 | 0.113 | 0.134 | 0.13 | 0.141 |
| A_52_P827911 | AK081941; | | | 2.387 | 2.657 | 2.47 | 2.075 | 2.44 | 2.116 |
| A_51_P231341 | AK087764; | 108160 | D0HXS9928E | 2.885 | 3.795 | 2.39 | 3.305 | 3.578 | 2.451 |
| A_51_P334093 | NM_026228 | 67547 | Slc39a8 | 0.208 | 0.0771 | 0.105 | 0.19 | 0.198 | 0.231 |
| A_52_P196568 | NM_144513 | 17263 | Gtl2 | 1.892 | 2.538 | 1.516 | 1.938 | 1.959 | 1.652 |
| A_52_P1108139 | AK053079; | | | 1.801 | 2.214 | 1.538 | 1.924 | 2.168 | 1.724 |
| A_52_P636559 | AK053008; | 268469 | 9530033F24Rik | 2.63 | 2.537 | 2.752 | 2.132 | 2.334 | 1.998 |
| A_52_P425734 | NM_145146 | 280662 | Afm | 3.913 | 3.546 | 3.74 | 3.764 | 4.135 | 3.335 |
| A_52_P1011737 | AK039096; | 106258 | AI790442 | 1.846 | 1.977 | 1.978 | 2.087 | 2.67 | 2.065 |
| A_52_P537140 | AK047235; | | | 1.74 | 2.015 | 2.044 | 1.613 | 1.708 | 1.261 |
| A_51_P191990 | AK087548; | 26563 | Ror1 | 1.628 | 1.623 | 1.626 | 1.509 | 1.588 | 1.479 |
| A_51_P478930 | AK080016; | | | 2.436 | 2.701 | 2.337 | 2.779 | 2.919 | 2.466 |
| A_51_P252565 | NM_009660 | 11687 | Alox15 | 0.236 | 0.24 | 0.156 | 0.217 | 0.231 | 0.212 |
| A_52_P81252 | NM_172599 | 218978 | D14Ertd436e | 2.478 | 2.667 | 2.607 | 1.82 | 1.981 | 1.764 |
| A_51_P487004 | NM_027732 | 71241 | Dmrtc2 | 10.3 | 11.69 | 10.98 | 5.024 | 6.192 | 5.685 |
| A_51_P104162 | AK053379; | 328232 | | 2.537 | 3.096 | 3.391 | 3.126 | 2.998 | 3.127 |
| A_52_P555603 | AK032770; | 11787 | Apbb2 | 1.409 | 1.222 | 1.29 | 1.326 | 1.427 | 1.18 |
| A_52_P819356 | AK041684; | | | 3.003 | 2.848 | 2.7 | 3.026 | 2.915 | 3.006 |
| A_52_P1196751 | AK086488; | | | 2.217 | 2.193 | 2.198 | 2.38 | 2.677 | 2.156 |
| A_52_P277415 | AK077313; | 76252 | 0610006O14Rik | 1.282 | 1.291 | 1.224 | 0.99 | 1.044 | 0.981 |
| A_52_P812362 | BC016205; | 408064 | BC064078 | 0.25 | 0.167 | 0.217 | 0.302 | 0.27 | 0.27 |
| A_51_P352572 | NM_019701 | 56365 | Clcnkb | 5.193 | 4.267 | 4.344 | 4.514 | 5.226 | 4.635 |
| A_52_P100042 | AK045143; | 320938 | Tnpo3 | 1.761 | 1.695 | 1.74 | 1.697 | 1.665 | 1.526 |
| A_51_P103397 | AK078521; | 54561 | Nap1l3 | 2.701 | 2.645 | 2.885 | 3.303 | 3.612 | 3.031 |
| A_52_P296244 | BC048734; | | | 0.369 | 0.373 | 0.292 | 0.457 | 0.42 | 0.426 |
| A_51_P305019 | AK020601; | 78423 | 9530049O05Rik | 1.297 | 1.416 | 1.484 | 1.43 | 1.551 | 1.387 |
| A_52_P3304 | NM_080446 | 117599 | Helb | 2.823 | 2.774 | 3.121 | 2.206 | 2.73 | 2.462 |
| A_51_P147651 | AK041797; XM_146705 | 245902 | | 2.797 | 2.965 | 2.694 | 2.18 | 2.325 | 1.652 |
| A_51_P203955 | NM_010260 | 14469 | Gbp2 | 0.252 | 0.267 | 0.292 | 0.582 | 0.578 | 0.664 |
| A_51_P176474 | AK010466; | 76484 | 2410012C07Rik | 2.797 | 2.691 | 2.733 | 3.87 | 4.072 | 3.726 |
| A_52_P165497 | NM_011385 | 20481 | Ski | 2.312 | 2.37 | 2.474 | 1.296 | 1.485 | 1.406 |
| A_51_P358714 | AK031433; | | | 2.043 | 2.448 | 1.68 | 2.647 | 2.627 | 1.899 |
| A_51_P106918 | NM_009051 | 19715 | Rex2 | 2.006 | 2.032 | 2.425 | 6.886 | 2.95 | 2.717 |
| A_51_P124315 | NM_177649 | 227712 | BC034076 | 1.796 | 1.742 | 1.797 | 2.396 | 2.68 | 2.14 |
| A_52_P183368 | NM_011535 | 21386 | Tbx3 | 1.964 | 1.81 | 1.931 | 2.023 | 2.272 | 2.026 |
| A_51_P140751 | NM_011397 | 20522 | Slc23a1 | 1.819 | 1.664 | 1.85 | 2.855 | 2.607 | 2.546 |
| A_51_P103406 | NM_011708 | 22371 | Vwf | 3.089 | 2.827 | 2.64 | 4.179 | 4.502 | 3.84 |
| A_51_P381069 | XM_132045 | 231252 | Chrna9 | 1.424 | 2.361 | 3.023 | 3.047 | 2.764 | 2.469 |
| A_52_P75971 | NM_026599 | 68178 | Cgnl1 | 1.56 | 1.535 | 1.702 | 1.265 | 1.357 | 1.222 |
| A_52_P135873 | XM_132015; XM_488584 | 231128 | BC037112 | 1.243 | 1.332 | 1.392 | 1.507 | 1.53 | 1.464 |
| A_51_P165082 | AB041601; XM_489523 | 224019 | D16Bwg1494e | 3.114 | 3.318 | 3.453 | 3.352 | 3.568 | 3.01 |
| A_51_P294566 | AK020687; | 78083 | 9930116N10Rik | 2.623 | 2.571 | 2.706 | 3.706 | 4.077 | 3.155 |
| A_51_P378051 | NM_009675 | 11754 | Aoc3 | 2.988 | 3.162 | 3.341 | 3.251 | 3.583 | 3.381 |
| A_52_P420608 | NM_201366 | 381371 | Gm1631 | 2.287 | 3.006 | 2.818 | 2.552 | 2.723 | 3.242 |
| A_51_P221291 | NM_009229 | 20650 | Sntb2 | 1.876 | 1.793 | 1.783 | 1.622 | 1.693 | 1.524 |
| A_52_P362805 | AK044730; | 269254 | Als4 | 2.262 | 2.248 | 2.26 | 1.818 | 1.967 | 1.718 |
| A_51_P109649 | AK018142; | 17263 | Gtl2 | 1.183 | 1.072 | 1.216 | 1.06 | 1.024 | 0.976 |
| A_52_P413348 | XM_127674 | 75698 | 3110001K24Rik | 2.072 | 2.063 | 2.27 | 1.908 | 2.161 | 1.733 |
| A_52_P400425 | NM_178656 | 193003 | A530088H08Rik | 2.498 | 2.036 | 2.006 | 2.674 | 2.834 | 2.576 |
| A_52_P147870 | NM_029963 | 77721 | Mrps5 | 2.14 | 2.028 | 2.027 | 1.789 | 1.827 | 1.856 |
| A_52_P57078 | AK052455; | | | 2.172 | 1.912 | 1.757 | 1.727 | 1.952 | 1.877 |
| A_52_P389874 | NM_011918 | 24131 | Ldb3 | 2.421 | 2.244 | 2.421 | 2.216 | 2.284 | 2.066 |
| A_52_P224125 | XM_622260 | | | 1.795 | 1.653 | 1.727 | 1.625 | 1.654 | 1.663 |
| A_52_P238468 | XM_133238 | 381853 | Gm160 | 2.307 | 2.173 | 2.136 | 1.929 | 1.906 | 1.683 |
| A_51_P253053 | XM_618867 | | | 2.544 | 3.669 | 3.053 | 2.284 | 2.658 | 3.007 |
| A_52_P500979 | NM_013628 | 18548 | Pcsk1 | 0.75 | 0.686 | 0.598 | 1.08 | 1.031 | 0.991 |
| A_51_P207934 | NM_199016 | 224794 | Enpp4 | 2.477 | 2.57 | 2.229 | 2.606 | 2.707 | 2.427 |
| A_52_P423384 | BC057551; XM_354546 | 104248 | Cabin1 | 1.58 | 1.963 | 1.524 | 1.442 | 1.596 | 1.426 |
| A_52_P560006 | NM_008426 | 16519 | Kcnj3 | 0.763 | 0.536 | 0.497 | 0.479 | 0.449 | 0.493 |
| A_52_P654624 | AK005333; | 236604 | 4933439C20Rik | 2.049 | 1.763 | 2.06 | 1.639 | 1.865 | 1.518 |
| A_52_P32502 | AK020822; | 77944 | A930007B11Rik | 2.593 | 4.558 | 2.534 | 2.973 | 3.231 | 2.237 |
| A_51_P302377 | NM_008009 | 14181 | Fgfbp1 | 0.258 | 0.246 | 0.273 | 0.413 | 0.381 | 0.451 |
| A_52_P255224 | AK032711; XM_486482 | 234371 | BC021367 | 2.31 | 3.108 | 2.526 | 2.817 | 2.716 | 2.5 |
| A_52_P571746 | XM_355533; XM_357399 | 230863 | LOC381571 | 5.455 | 5.043 | 4.955 | 4.478 | 4.627 | 3.701 |
| A_52_P458916 | AK016751; | 218850 | D14Abb1e | 2.421 | 2.013 | 2.081 | 2.688 | 2.359 | 2.992 |
| A_52_P286970 | XM_131162; XM_127565 | 76561 | Snx7 | 0.44 | 0.492 | 0.435 | 0.608 | 0.611 | 0.895 |
| A_51_P266644 | BC055753; XM_125538 | 140742 | | 0.424 | 0.369 | 0.474 | 0.821 | 0.739 | 0.949 |
| A_52_P233673 | NM_020594 | 57432 | Zc3hdc8 | 3.001 | 3.71 | 2.727 | 1.913 | 2.05 | 1.886 |
| A_52_P1084149 | AK051413; | 553138 | 9930021J03Rik | 1.669 | 1.913 | 1.945 | 1.806 | 2.202 | 1.595 |
| A_52_P413661 | BC079861; XM_484162 | 217716 | Mlh3 | 2.28 | 2.065 | 2.239 | 2.466 | 2.362 | 2.456 |
| A_51_P404193 | NM_022435 | 64406 | Sp5 | 0.194 | 0.182 | 0.19 | 0.214 | 0.202 | 0.207 |
| A_52_P565940 | AF419220; | 18193 | Nsd1 | 1.84 | 1.873 | 1.944 | 1.445 | 1.546 | 1.33 |
| A_51_P514177 | NM_028838 | 74249 | Lrrc2 | 3.542 | 3.2 | 3.896 | 3.884 | 4.114 | 3.603 |
| A_52_P237357 | AF095690; | | | 1.813 | 1.675 | 1.846 | 1.603 | 1.706 | 1.432 |
| A_51_P240811 | NM_009290 | 20890 | Wnt8a | 0.709 | 0.589 | 0.527 | 1.532 | 0.979 | 1.578 |
| A_51_P236267 | NM_009183 | 20452 | St8sia4 | 0.265 | 0.182 | 0.148 | 0.233 | 0.291 | 0.32 |
| A_52_P405340 | U76762; | 14158 | Fert2 | 2.538 | 2.399 | 2.414 | 2.011 | 2.124 | 1.824 |
| A_52_P661144 | XM_485365 | | | 2.066 | 2.37 | 2.399 | 2.3 | 2.365 | 2.13 |
| A_52_P1044262 | AK051949; | | | 1.478 | 1.257 | 1.19 | 1.611 | 1.769 | 1.41 |
| A_52_P553100 | AK040413; | | | 2.967 | 3.025 | 3.491 | 3.19 | 3.168 | 2.79 |
| A_51_P358344 | NM_007732 | 12821 | Col17a1 | 3.116 | 2.917 | 3.011 | 3.334 | 3.243 | 2.913 |
| A_51_P321579 | XM_131865; XM_196334 | 433809 | LOC433809 | 2.839 | 2.589 | 2.323 | 1.805 | 1.942 | 1.521 |
| A_52_P485939 | NM_026228 | 67547 | Slc39a8 | 0.407 | 0.418 | 0.26 | 0.31 | 0.338 | 0.278 |
| A_52_P199922 | | | | 3.785 | 1.908 | 2.417 | 2.334 | 2.502 | 2.545 |
| A_51_P463791 | BC051947; | 58212 | | 3.074 | 2.866 | 2.865 | 2.058 | 2.064 | 1.757 |
| A_52_P487362 | NM_028980 | 74521 | 8430415E04Rik | 0.317 | 0.316 | 0.334 | 0.477 | 0.454 | 0.469 |
| A_51_P341137 | AK035176; | 224829 | Trerf1 | 1.935 | 1.833 | 2.021 | 2.024 | 2.334 | 1.984 |
| A_51_P407323 | NM_007976 | 14067 | F5 | 0.17 | 0.072 | NO DATA | 0.352 | 0.305 | 0.242 |
| A_51_P272363 | NM_134160 | 171166 | Mcoln3 | 0.559 | 0.82 | 0.792 | 0.686 | 0.762 | 0.915 |
| A_52_P430927 | AK042385; | 11908 | Atf1 | 1.603 | 1.64 | 1.71 | 1.368 | 1.604 | 1.26 |
| A_52_P626069 | AK040994; XM_284439 | 109151 | Chd9 | 1.61 | 3.967 | 1.955 | 2.209 | 2.26 | 1.715 |
| A_52_P355276 | NM_001002764 | 103677 | AI317223 | 2.549 | 2.993 | 2.548 | 1.703 | 1.935 | 1.511 |
| A_52_P650215 | NM_029278 | 75416 | 2610033H07Rik | 1.439 | 4.127 | 1.948 | 2.359 | 2.484 | 1.577 |
| A_52_P416123 | AK020480; | 77358 | 9430069I07Rik | 2.511 | 2.833 | 2.298 | 1.785 | 1.993 | 1.656 |
| A_51_P189631 | NM_172427 | 76455 | 2310067E19Rik | 1.139 | 0.941 | 0.985 | 1.62 | 1.653 | 1.604 |
| A_52_P16973 | AK081934; | 320391 | D930017J03Rik | 2.586 | 2.277 | 2.281 | 2.137 | 2.272 | 1.872 |
| A_52_P947879 | AK048751; | | | 1.687 | 1.729 | 1.647 | 2.149 | 2.232 | 2.009 |
| A_52_P538515 | BC049181; | 226412 | R3hdm | 2.219 | 2.049 | 2.456 | 1.981 | 2.364 | 1.119 |
| A_52_P179250 | AK129127; | 230249 | AI314180 | 5.166 | 6.292 | 4.565 | 8.052 | 10.48 | 5.993 |
| A_52_P795628 | AK086352; | | | 4.861 | 5.344 | 4.508 | 6.792 | 7.236 | 5.913 |
| A_51_P144712 | NM_178930 | 107338 | Gbf1 | 1.805 | 1.74 | 1.841 | 1.291 | 1.456 | 1.202 |
| A_52_P655008 | AK044382; | 320678 | A930037G23Rik | 1.653 | 1.779 | 1.572 | 1.52 | 1.586 | 1.325 |
| A_52_P577072 | NM_011901 | 24074 | Taf7 | 2.198 | 2.692 | 2.144 | 1.999 | 2.035 | 1.851 |
| A_52_P636199 | NM_008017 | 14211 | Smc2l1 | 1.61 | 2.123 | 1.631 | 1.531 | 1.607 | 1.38 |
| A_51_P447189 | AK041326; XM_358090 | 385154 | | 2.336 | 2.35 | 2.641 | 3.32 | 2.976 | 3.108 |
| A_52_P1180315 | AK049088; | 74022 | 3930401K13Rik | 1.789 | 1.737 | 1.505 | 1.313 | 1.364 | 1.207 |
| A_52_P412167 | NM_080467 | 140494 | Atp6v0a4 | 2.324 | 2.449 | 2.406 | 4.276 | 4.719 | 3.334 |
| A_52_P335089 | BE198552; | | | 1.331 | 1.348 | 1.455 | 1.151 | 1.229 | 0.977 |
| A_52_P6621 | | | | 2.585 | 4.954 | 2.769 | 4.277 | 4.565 | 2.653 |
| A_51_P343129 | AK012079; | 70472 | Atad2 | 3.752 | 4.107 | 4.116 | 2.307 | 2.496 | 1.87 |
| A_51_P144417 | NM_181750 | 226412 | R3hdm | 2.79 | 2.726 | 2.677 | 2.283 | 2.582 | 2.164 |
| A_52_P350477 | NM_139295 | 193813 | Mcfd2 | 0.43 | 0.349 | 0.245 | 0.661 | 0.554 | 0.759 |
| A_51_P418884 | NM_001013789 | 432823 | | 1.678 | 2.207 | 2.11 | 2.759 | 2.736 | 2.272 |
| A_52_P581138 | NM_212450 | 329506 | D2Ertd485e | 1.847 | 1.821 | 1.932 | 1.418 | 1.573 | 1.284 |
| A_52_P434549 | NM_023190 | 56215 | Acin1 | 1.868 | 1.876 | 1.953 | 1.241 | 1.454 | 1.123 |
| A_52_P384392 | AK080827; | 328087 | D930001B02 | 2.365 | 2.375 | 2.393 | 1.135 | 1.269 | 1.098 |
| A_51_P270725 | AK089493; | 102093 | Phkb | 2.083 | 3.565 | 2.709 | 2.953 | 2.589 | 2.102 |
| A_51_P193019 | NM_011157 | 19073 | Prg1 | 1.283 | 1.107 | 1.495 | 0.759 | 3.853 | 1.325 |
| A_52_P1172548 | AK084146; | 13204 | Dhx15 | 2.924 | 3.082 | 2.339 | 3.164 | 3.492 | 2.732 |
| A_51_P114606 | NM_016965 | 50884 | Nckap1 | 0.446 | 0.473 | 0.337 | 0.707 | 0.582 | 0.773 |
| A_52_P306387 | NM_178709 | 235315 | D130054N24Rik | 2.399 | 3.176 | 2.415 | 1.899 | 2.109 | 1.481 |
| A_52_P287576 | AK036308; | | | 1.703 | 2.271 | 1.347 | 2.425 | 2.794 | 1.7 |
| A_52_P501767 | AK012272; | 20637 | Snrp70 | 1.314 | 1.333 | 1.34 | 0.962 | 1 | 0.898 |
| A_52_P995717 | AK045222; | | | 3.292 | 3.495 | 3.303 | 2.04 | 2.108 | 1.729 |

TABLE 2B
292 Gene Set (Non Germline Comeptent)

| | | | | Clone 1 | | | Clone 2 | | |
|---|---|---|---|---|---|---|---|---|---|
| Agilent Probe ID | Genbank | Entrez ID | Gene Symbol | rep. 1 | rep. 2 | rep. 3 | rep. 1 | rep. 2 | rep. 3 |
| A_51_P237040 | NM_008711 | 18121 | Nog | 1.152 | 1.407 | 1.101 | 0.859 | 0.96 | 0.924 |
| A_51_P335460 | NM_009132 | 20259 | Scin | 1.658 | 1.913 | 1.519 | 1.101 | 1.225 | 1.107 |
| A_52_P391505 | NM_144513 | 17263 | Gtl2 | 0.0793 | 0.114 | 0.0867 | 0.158 | 0.15 | 0.148 |
| A_51_P497100 | NM_010706 | 16855 | Lgals4 | 0.692 | 0.771 | 0.849 | 1.002 | 0.908 | 0.828 |
| A_51_P378371 | | | | 0.986 | 1.006 | 1.208 | 1.217 | 1.156 | 1.234 |
| A_51_P298266 | NM_025681 | 66643 | Lix1 | 1.123 | 1.145 | 1.109 | 1.143 | 1.15 | 1.141 |
| A_52_P269158 | NM_001003948 | 98496 | AL024069 | 1.775 | 1.419 | 1.506 | 1.494 | 1.391 | 1.254 |
| A_51_P311038 | BC009666; XM_134720 | 57267 | Apba3 | 1.847 | 1.642 | 1.538 | 2.55 | 2.405 | 2.808 |
| A_51_P457187 | NM_028075 | 72049 | Tnfrsf13c | 1.083 | 1.58 | 1.373 | 0.916 | 1.099 | 1.091 |
| A_52_P68702 | NM_145148 | 232288 | Frmd4b | 0.928 | 1.071 | 0.877 | 1.242 | 1.234 | 1.139 |
| A_52_P293443 | AK087208; | 13819 | | 0.179 | 0.222 | 0.219 | 0.543 | 0.565 | 0.552 |
| A_51_P157986 | NM_029930 | 77574 | 3321401G04Rik | 0.909 | 1.196 | 1.088 | 0.563 | 0.621 | 0.598 |
| A_52_P1013441 | | | | 1.255 | 1.032 | 2.041 | 1.173 | 1.203 | 1.341 |
| A_51_P418375 | NM_023844 | 67374 | Jam2 | 1.044 | 0.943 | 0.99 | 0.826 | 0.786 | 0.84 |
| A_52_P557940 | AK004302; | 66704 | 4921506I22Rik | 0.544 | 0.595 | 0.569 | 1.371 | 1.379 | 1.241 |
| A_51_P480311 | NM_010168 | 14061 | F2 | 0.348 | 0.476 | 0.622 | 0.996 | 1.004 | 1.186 |
| A_52_P200458 | NM_010242 | 14345 | Fut4 | 1.003 | 0.997 | 1.017 | 1.108 | 1.203 | 1.09 |
| A_51_P463846 | NM_145545 | 229900 | 9830147J24Rik | 2.28 | 2.199 | 2.49 | 2.068 | 1.958 | 1.72 |
| A_51_P162474 | AF285577; | 107815 | Scml2 | 2.885 | 2.549 | 2.371 | 3.021 | 3.116 | 3.1 |
| A_51_P165244 | NM_018734 | 55932 | Gbp4 | 2.388 | 1.319 | 2.543 | 2.404 | 2.051 | 2.534 |
| A_52_P59318 | NM_133832 | 98711 | Rdh10 | 1.689 | 1.611 | 1.584 | 0.876 | 1.002 | 0.955 |
| A_52_P438036 | NM_175271 | 78134 | Gpr23 | 1.082 | 0.96 | 0.968 | 1.634 | 1.593 | 1.618 |
| A_52_P474242 | BE457768; | | | 1.31 | 1.785 | 1.55 | 1.137 | 1.132 | 0.997 |
| A_51_P499854 | NM_010284 | 14600 | Ghr | 1.55 | 1.19 | 1.486 | 2.502 | 2.537 | 2.196 |
| A_51_P455647 | NM_009801 | 12349 | Car2 | 1.592 | 1.463 | 1.337 | 2.139 | 2.347 | 2.17 |
| A_52_P103929 | NM_009306 | 20979 | Syt1 | 0.898 | 0.938 | 0.967 | 1.626 | 1.464 | 1.422 |
| A_51_P149469 | NM_009608 | 11464 | Actc1 | 1.012 | 1.006 | 0.935 | 0.971 | 0.997 | 1.12 |
| A_52_P557293 | NM_015775 | 50528 | Tmprss2 | 1.142 | 1.091 | 1.111 | 1.208 | 1.127 | 1.225 |
| A_52_P50496 | AY048587; XM_619453 | 114640 | Tifp39 | 1.196 | 1.551 | 1.504 | 1.114 | 1.171 | 1.214 |
| A_51_P180413 | NM_176930 | 319504 | C130076O07Rik | 1.603 | 1.54 | 1.395 | 1.297 | 1.25 | 1.155 |
| A_51_P229613 | NM_008792 | 18549 | Pcsk2 | 1.463 | 1.53 | 1.37 | 1.333 | 1.422 | 1.554 |
| A_51_P121635 | NM_030700 | 80884 | Maged2 | 1.225 | 1.426 | 1.335 | 0.918 | 0.99 | 0.88 |
| A_51_P481777 | NM_018756 | 54382 | Tcstv1 | 0.208 | 0.696 | 0.654 | 0.592 | 0.503 | 0.72 |
| A_51_P412817 | NM_177617 | 216851 | D330014H01Rik | 0.725 | 0.834 | 1.04 | 1.083 | 0.986 | 0.997 |
| A_51_P387528 | AK019664; | 78801 | Ak7 | 0.65 | 0.537 | 0.813 | 0.767 | 0.778 | 1.11 |
| A_52_P78168 | AK031518; | | | 0.277 | 0.385 | 0.299 | 0.704 | 0.652 | 0.544 |
| A_52_P274960 | NM_001005508 | 226652 | 6030405P05Rik | 0.671 | 0.634 | 0.712 | 0.763 | 0.781 | 0.808 |
| A_52_P1188270 | AK042362; | | | 0.341 | 0.594 | 0.564 | 1.758 | 1.462 | 1.658 |
| A_51_P291227 | NM_001005508 | 226652 | 6030405P05Rik | 0.705 | 0.681 | 0.731 | 0.814 | 0.735 | 0.798 |
| A_52_P377299 | AK048360; | 271377 | Zbtb11 | 0.577 | 0.582 | 0.645 | 1.152 | 1.092 | 1.023 |
| A_51_P297131 | NM_009502 | 22330 | Vcl | 1.015 | 1.436 | 0.955 | 0.556 | 0.672 | 0.587 |
| A_52_P804224 | | | | 0.299 | 0.314 | 0.291 | 1.263 | 1.2 | 1.068 |
| A_51_P442894 | AK048310; | 621603 | C130048D07Rik | 1.023 | 0.841 | 1.218 | 0.788 | 0.867 | 0.977 |
| A_52_P309718 | AK173099; | 218850 | D14Abb1e | 1.006 | 0.813 | 1.127 | 1.186 | 1.29 | 1.289 |
| A_52_P359010 | AK129478; | 72193 | Sfrs2ip | 0.54 | 0.706 | 0.574 | 1.484 | 1.398 | 1.403 |
| A_51_P410813 | AK079732; | | | 0.717 | 0.877 | 0.904 | 1.593 | 1.473 | 1.607 |
| A_52_P382565 | XM_483943 | 432493 | LOC432493 | 0.228 | 0.273 | 0.254 | 0.607 | 0.622 | 0.462 |
| A_51_P333253 | NM_178440 | 246177 | Myo1g | 0.535 | 0.491 | 0.538 | 0.474 | 0.481 | 0.37 |
| A_52_P113190 | NM_053214 | 17916 | Myo1f | 0.386 | 0.513 | 0.397 | 0.243 | 0.232 | 0.264 |
| A_51_P473953 | XM_358600; | 381498 | 4930592C13Rik | 2.138 | 1.593 | 1.947 | 2.323 | 2.226 | 2.26 |
| | XM_355454 | | | | | | | | |
| A_52_P519870 | AB010340; | | | 0.758 | 0.675 | 1.128 | 0.989 | 0.863 | 1.393 |
| A_51_P266403 | AK009163; | 76730 | 2310005C01Rik | 0.984 | 0.901 | 1.009 | 1.273 | 1.313 | 1.391 |
| A_52_P3698 | NM_001015099 | 217558 | | 0.658 | 0.653 | 0.676 | 1.487 | 1.564 | 1.537 |
| A_52_P581100 | NM_027285 | 70005 | 1700029I01Rik | 1.164 | 1.1 | 1.141 | 1.525 | 1.383 | 1.305 |
| A_51_P276305 | NM_009292 | 20899 | Stra8 | 0.873 | 0.934 | 0.982 | 0.431 | 0.426 | 0.471 |
| A_51_P455997 | Y13832; | 17263 | Gtl2 | 0.123 | 0.184 | 0.114 | 0.213 | 0.197 | 0.258 |
| A_52_P656336 | NM_023733 | 74114 | Crot | 1.233 | 1.547 | 1.442 | 0.983 | 1.067 | 0.998 |
| A_51_P512172 | AK010953; XM_125673 | 52463 | Cxxc6 | 0.8 | 0.986 | 0.748 | 1.033 | 1.014 | 1.046 |
| A_51_P282227 | NM_020577 | 57344 | As3mt | 0.79 | 0.761 | 0.777 | 1.02 | 0.91 | 0.98 |
| A_52_P386544 | NM_026594 | 68172 | 4930517K11Rik | 0.74 | 0.606 | 0.729 | 1.188 | 1.159 | 1.351 |
| A_51_P125679 | AK010725; XM_134948 | 73673 | 2410076I21Rik | 0.588 | 0.959 | 0.916 | 0.84 | 0.843 | 0.849 |
| A_51_P164504 | NM_007469 | 11812 | Apoc1 | 1.307 | 1.245 | 1.314 | 1.412 | 1.375 | 1.457 |
| A_52_P240706 | NM_007543 | 26367 | Ceacam2 | 0.87 | 0.778 | 0.892 | 0.689 | 0.756 | 0.859 |
| A_52_P420369 | NM_029444 | 75801 | 4930447C04Rik | 1.087 | 1.084 | 1.129 | 1.176 | 0.989 | 1.041 |
| A_51_P242399 | NM_031170 | 16691 | Krt2-8 | 1.013 | 1.199 | 0.939 | 0.962 | 0.977 | 0.946 |
| A_51_P158037 | NM_013505 | 13506 | Dsc2 | 1.238 | 1.562 | 1.304 | 0.743 | 0.819 | 0.875 |
| A_52_P258116 | NM_009521 | 22415 | Wnt3 | 1.304 | 1.249 | 1.083 | 1.811 | 1.943 | 2.006 |
| A_52_P286166 | NM_001002927 | 18619 | Penk1 | 1.649 | 2.509 | 2.439 | 1.542 | 1.453 | 1.374 |
| A_52_P399095 | NM_177793 | 327747 | 9030224M15Rik | 1.11 | 0.976 | 1.056 | 1.583 | 1.692 | 1.596 |
| A_52_P470663 | | | | 2.339 | 2.72 | 2.405 | 1.069 | 1.105 | 1.018 |
| A_52_P376169 | NM_177139 | 320343 | E130115E03Rik | 1.184 | 1.025 | 1.076 | 1.949 | 1.81 | 1.828 |
| A_51_P512085 | NM_130449 | 140792 | Colec12 | 1.574 | 1.25 | 1.471 | 1.463 | 1.651 | 1.693 |
| A_52_P676403 | NM_019494 | 56066 | Cxcl11 | 3.86 | 3.177 | 3.663 | 2.834 | 2.723 | 2.826 |
| A_51_P354307 | NM_172469 | 209195 | Clic6 | 1.31 | 1.578 | 1.57 | 0.719 | 0.748 | 0.659 |
| A_52_P569375 | NM_010203 | 14176 | Fgf5 | 4.338 | 3.206 | 3.958 | 5.889 | 5.927 | 6.75 |
| A_51_P162481 | NM_133194 | 107815 | Scml2 | 2.434 | 2.616 | 2.232 | 2.312 | 2.394 | 2.596 |
| A_51_P202911 | NM_144882 | 67198 | 2810022L02Rik | 1.005 | 0.995 | 0.908 | 0.913 | 0.931 | 0.902 |
| A_51_P418029 | NM_175541 | 245631 | Mum1l1 | 2.026 | 1.803 | 2.159 | 1.492 | 1.654 | 1.714 |
| A_52_P93837 | NM_008604 | 17380 | Mme | 1.516 | 1.805 | 1.761 | 1.442 | 1.369 | 1.329 |
| A_51_P275435 | NM_172411 | 71874 | 2310007B03Rik | 3.355 | 3.105 | 3.53 | 5.636 | 6.155 | 6.288 |
| A_51_P199888 | NM_026082 | 67299 | Dock7 | 1.182 | 1.246 | 1.295 | 0.669 | 0.678 | 1 |
| A_52_P211956 | NM_025658 | 66607 | Ms4a4d | 1.71 | 1.799 | 1.851 | 2.105 | 2.274 | 1.961 |
| A_51_P485985 | NM_134163 | 171170 | Mbnl3 | 1.781 | 1.802 | 1.648 | 0.929 | 1.067 | 1.035 |
| A_52_P630964 | NM_027533 | 70747 | Tspan2 | 1.217 | 1.314 | 1.26 | 0.815 | 0.863 | 0.897 |
| A_51_P294505 | NM_025658 | 66607 | Ms4a4d | 1.546 | 1.551 | 1.883 | 2.127 | 2.066 | 2.183 |
| A_52_P82488 | NM_021476 | 58861 | Cysltr1 | 1.919 | 1.474 | 1.811 | 2.106 | 2.164 | 2.621 |
| A_52_P634829 | NM_023835 | 76681 | Trim12 | 1.52 | 1.676 | 1.775 | 1.472 | 1.69 | 1.577 |
| A_52_P460882 | XM_130319; | 73668; 14725 | Ttc21b | 1.498 | 1.698 | 2.077 | 1.06 | 1.276 | 1.219 |
| | XM_130308 | | | | | | | | |
| A_52_P197826 | NM_010397 | 15039 | H2-T22 | 1.502 | 1.687 | 1.444 | 1.028 | 1.049 | 1.039 |
| A_51_P240253 | NM_019662 | 56437 | Rrad | 1.392 | 1.648 | 1.29 | 0.851 | 0.9 | 0.913 |
| A_51_P101975 | NM_010205 | 14179 | Fgf8 | 2.683 | 2.96 | 2.139 | 3.25 | 3.463 | 3.854 |
| A_52_P21595 | NM_028238 | 72433 | Rab38 | 1.043 | 0.791 | 0.992 | 0.807 | 0.819 | 0.929 |
| A_52_P402786 | NM_008935 | 19126 | Prom1 | 1.498 | 1.71 | 1.749 | 1.028 | 1.023 | 0.92 |
| A_52_P126158 | NM_008326 | 15944 | Ifi1 | 1.157 | 1.034 | 1.006 | 1.254 | 1.3 | 1.232 |
| A_52_P676510 | NM_011579 | 21822 | Tgtp | 1.166 | 1.03 | 1.064 | 1.15 | 1.119 | 1.082 |
| A_52_P175685 | NM_008390 | 16362 | Irf1 | 1.072 | 1.33 | 1.162 | 0.971 | 1.106 | 1.017 |
| A_52_P384718 | BC052056; XM_283264 | 320873 | | 1.488 | 1.451 | 1.435 | 1.051 | 1.032 | 1.026 |
| A_52_P176333 | NM_001005787 | 12695 | Cipp | 1.168 | 1.146 | 1.249 | 0.974 | 1.014 | 1.036 |
| A_52_P358951 | AK081999; XM_134736 | 271457; 22688 | Rab5a | 1.346 | 1.445 | 1.326 | 0.843 | 0.883 | 0.787 |
| A_51_P168646 | AK039580; | | | 1.655 | 1.373 | 1.436 | 2.357 | 2.478 | 2.267 |
| A_52_P620345 | NM_133979 | 102566 | AI604832 | 1.26 | 1.488 | 1.375 | 0.778 | 0.752 | 0.729 |
| A_52_P253179 | AK077477; | 16009 | Igfbp3 | 2.157 | 1.536 | 2.035 | 1.993 | 1.965 | 2.545 |
| A_51_P397003 | XM_133990 | 68904 | 1110065L07Rik | 1.235 | 1.343 | 1.238 | 0.778 | 0.763 | 0.855 |
| A_52_P74441 | NM_011570 | 21753 | Tes | 1.498 | 1.167 | 1.425 | 0.853 | 0.882 | 0.885 |
| A_52_P255250 | NM_009543 | 22644 | Rnf103 | 1.205 | 1.217 | 1.524 | 0.663 | 0.701 | 0.687 |
| A_52_P575505 | NM_008994 | 19302 | Pxmp3 | 1.123 | 1.07 | 1.092 | 0.797 | 0.815 | 0.802 |
| A_52_P469789 | NM_007588 | 12311 | Calcr | 2.087 | 2.206 | 2.278 | 1.646 | 1.638 | 1.533 |
| A_51_P345422 | NM_010747 | 17096 | Lyn | 0.888 | 0.803 | 0.881 | 1.006 | 0.995 | 0.89 |
| A_51_P356003 | NM_001007580 | 333564 | Gm784 | 1.223 | 1.221 | 1.186 | 1.543 | 1.618 | 1.409 |
| A_52_P680941 | NM_010284 | 14600 | Ghr | 1.72 | 1.563 | 1.682 | 2.058 | 1.978 | 2.065 |
| A_52_P380263 | NM_013723 | 27205 | Podxl | 1.179 | 1.159 | 1.66 | 0.899 | 0.909 | 0.942 |
| A_52_P286528 | BC055771; | 102954 | Nudt11 | 1.419 | 1.344 | 1.431 | 1.048 | 1.063 | 1.053 |
| A_51_P105068 | NM_027990 | 71897 | 2310010M24Rik | 1.902 | 1.982 | 1.72 | 2.012 | 2.165 | 2.002 |
| A_52_P132608 | NM_172685 | 229731 | Slc25a24 | 0.809 | 0.906 | 0.807 | 0.708 | 0.722 | 0.693 |
| A_51_P467505 | NM_013689 | 21682 | Tec | 1.077 | 1.062 | 1.153 | 0.842 | 0.861 | 0.881 |
| A_52_P153685 | AK129323; | 70422 | 2810417D08Rik | 1.763 | 1.676 | 1.845 | 2.204 | 2.099 | 2.085 |
| A_51_P174943 | NM_008485 | 16782 | Lamc2 | 1.282 | 1.255 | 1.249 | 1.068 | 1.04 | 1.004 |
| A_52_P301821 | NM_053078 | 27528 | D0H4S114 | 1.076 | 1.275 | 1.138 | 0.88 | 0.828 | 0.741 |
| A_52_P532910 | NM_024427 | 22003 | Tpm1 | 1.081 | 1.106 | 1.238 | 0.891 | 0.87 | 0.851 |
| A_51_P521084 | NM_025626 | 66540 | 3110001A13Rik | 1.423 | 1.425 | 1.474 | 0.832 | 0.865 | 0.776 |
| A_52_P680436 | NM_025793 | 66840 | Wdr45l | 1.338 | 1.213 | 1.346 | 0.755 | 0.702 | 0.805 |
| A_51_P164219 | NM_011909 | 24110 | Usp18 | 1.876 | 1.698 | 1.64 | 1.445 | 1.463 | 1.349 |
| A_52_P421234 | NM_133832 | 98711 | Rdh10 | 1.706 | 1.519 | 1.676 | 0.845 | 0.856 | 0.776 |
| A_52_P586157 | NM_022309 | 12400 | Cbfb | 1.417 | 1.551 | 1.338 | 0.773 | 0.863 | 0.903 |
| A_51_P262171 | NM_008326 | 15944 | Ifi1 | 1.271 | 1.011 | 1.135 | 1.457 | 1.571 | 1.485 |
| A_52_P452689 | NM_007498 | 11910 | Atf3 | 0.672 | 0.61 | 0.659 | 0.89 | 0.955 | 0.943 |
| A_52_P295933 | AK045060; | 14051 | B130023L16Rik | 1.637 | 1.973 | 2.08 | 1.007 | 0.993 | 1.019 |
| A_51_P222773 | NM_010446 | 15376 | Foxa2 | 2.333 | 1.693 | 1.64 | 6.867 | 6.289 | 6.137 |
| A_52_P361534 | NM_009521 | 22415 | Wnt3 | 1.193 | 0.891 | 0.969 | 2.247 | 2.459 | 2.231 |
| A_51_P507880 | NM_028980 | 74521 | 8430415E04Rik | 2.104 | 2.169 | 2.081 | 2.44 | 2.499 | 2.391 |
| A_52_P574668 | NM_011851 | 23959 | Nt5e | 1.611 | 1.127 | 1.284 | 1.764 | 1.707 | 1.826 |
| A_52_P957500 | XM_622354 | | | 0.371 | 0.532 | 0.397 | 1.528 | 1.372 | 1.305 |
| A_52_P345626 | AY149175; | 386626 | Mzf6d | 0.699 | 0.636 | 0.76 | 1.173 | 1.052 | 1.267 |
| A_52_P84015 | AK037762; | 11863 | | 0.695 | 0.924 | 0.842 | 2.965 | 3.344 | 3.023 |
| A_52_P432464 | AK053863; | 192196 | Luc7l2 | 0.7 | 0.863 | 1.032 | 1.457 | 1.269 | 1.582 |
| A_52_P376502 | BC025011; | 227541 | Camk1d | 0.664 | 0.71 | 0.737 | 0.973 | 0.893 | 1.008 |
| A_52_P354785 | AK037929; | 14852 | Gspt1 | 0.487 | 0.598 | 0.585 | 1.587 | 1.447 | 1.483 |
| A_51_P398766 | NM_010259 | 14468 | Gbp1 | 3.558 | 2.476 | 3.155 | 4.099 | 3.953 | 3.971 |
| A_52_P486234 | AK033497; | 22680 | | 0.658 | 0.943 | 0.756 | 2.133 | 2.185 | 1.908 |
| A_52_P628212 | AK044339; | 26380 | | 1.058 | 1.337 | 0.967 | 4.002 | 3.276 | 3.435 |
| A_52_P445799 | AK046777; | 52490 | D18Ertd289e | 2.088 | 2.22 | 2.899 | 2.952 | 2.963 | 3.531 |
| A_51_P268167 | BC037232; XM_621365 | 229363; 218850 | Gmps | 0.465 | 0.448 | 0.523 | 1.267 | 1.053 | 1.205 |
| A_51_P153079 | NM_133923 | 101100 | 4833441J24Rik | 0.42 | 0.232 | 0.278 | 1.153 | 1.158 | 1.047 |
| A_52_P584874 | NM_152895 | 75605 | Jarid1b | 0.737 | 0.83 | 0.752 | 1.242 | 1.251 | 1.242 |
| A_52_P307801 | NM_007847 | 13222 | Defcr-rs2 | 2.149 | 1.602 | 1.28 | 2.942 | 3.1 | 2.825 |
| A_52_P163148 | AB010370; | 544945 | | 0.704 | 0.687 | 0.76 | 1.098 | 1.144 | 1.325 |
| A_51_P171728 | NM_011926 | 26365 | Ceacam1 | 0.474 | 0.703 | 0.686 | 0.748 | 0.952 | 0.943 |
| A_51_P141660 | AK053665; XM_358382 | 67776; 235380 | Loh11cr2a | 0.43 | 0.62 | 0.908 | 1.844 | 1.422 | 1.976 |
| A_52_P532323 | NM_178410 | 218518 | Marveld2 | 1.824 | 2.102 | 1.904 | 0.871 | 0.884 | 1.027 |
| A_51_P337125 | NM_010566 | 16331 | Inpp5d | 0.938 | 0.873 | 1.036 | 1.261 | 1.272 | 1.297 |
| A_52_P336259 | AK087367; XM_485576 | 319467 | A930002C04Rik | 0.552 | 0.507 | 0.623 | 1.053 | 0.913 | 0.8 |
| A_51_P318577 | AK010729; | 73671 | 2410078J06Rik | 0.605 | 0.863 | 0.641 | 0.897 | 1.028 | 1.15 |
| A_52_P324934 | AK045391; | 16403 | | 0.758 | 0.573 | 0.774 | 1.808 | 1.513 | 1.534 |
| A_52_P826666 | AK007019; | 51799 | Rap2ip | 0.894 | 1.131 | 1.14 | 2.913 | 2.479 | 3.106 |
| A_51_P200223 | XM_619497 | | | 0.65 | 0.646 | 0.76 | 1.392 | 1.367 | 1.332 |
| A_51_P228472 | NM_008343 | 16009 | Igfbp3 | 1.472 | 2.26 | 1.637 | 1.02 | 1.11 | 1.239 |
| A_52_P206573 | NM_027427 | 70439 | Taf15 | 0.525 | 0.7 | 0.806 | 1.685 | 1.521 | 1.76 |
| A_52_P354363 | NM_026574 | 68142 | | 0.545 | 0.546 | 0.579 | 1.246 | 1.113 | 1.072 |
| A_52_P651469 | NM_001012726 | 503692 | | 1.087 | 2.041 | 1.585 | 2.114 | 1.954 | 2.198 |
| A_51_P463789 | BC051947; | 58212 | | 0.924 | 0.962 | 0.995 | 1.518 | 1.581 | 1.46 |
| A_52_P18887 | NM_011468 | 20755 | Sprr2a | 7.008 | 6.235 | 5.038 | 6.207 | 6.689 | 6.331 |
| A_51_P223132 | NM_031183; | 83395; 70549 | Sp6 | 0.649 | 0.542 | 0.552 | 1.095 | 0.998 | 1.125 |
| | XM_486227 | | | | | | | | |
| A_52_P223083 | BC038323; | 72096 | 2010208K18Rik | 0.89 | 0.64 | 1.018 | 2.024 | 1.641 | 2.309 |
| A_52_P523399 | NM_029444 | 75801 | 4930447C04Rik | 1.167 | 0.873 | 1.364 | 1.373 | 1.087 | 1.771 |
| A_52_P282000 | AK020248; | 13800 | Enah | 0.368 | 0.482 | 0.376 | 1.791 | 1.63 | 1.536 |
| A_52_P152983 | AK011739; | 26886 | Cenph | 0.423 | 0.805 | 0.28 | 1.393 | 1.582 | 1.833 |
| A_52_P26416 | AF106279; | 16782 | | 1.436 | 1.802 | 1.779 | 0.723 | 0.806 | 0.697 |
| A_51_P102081 | NM_007847 | 13222 | Defcr-rs2 | 2.408 | 2.238 | 1.498 | 3.38 | 3.255 | 2.763 |
| A_51_P285725 | NM_009309 | 20997 | T | 2.672 | 1.6 | 1.512 | 3.129 | 3.214 | 3.267 |
| A_52_P827911 | AK081941; | | | 0.209 | 0.476 | 0.487 | 1.73 | 1.566 | 1.778 |
| A_51_P231341 | AK087764; | 108160 | D0HXS9928E | 0.131 | 0.284 | 0.334 | 1.584 | 1 | 1.654 |
| A_51_P334093 | NM_026228 | 67547 | Slc39a8 | 2.173 | 2.638 | 2.466 | 1.07 | 1.006 | 1.061 |
| A_52_P196568 | NM_144513 | 17263 | Gtl2 | 0.0697 | 0.0369 | 0.0417 | 0.112 | 0.08 | 0.115 |
| A_52_P1108139 | AK053079; | | | 0.169 | 0.348 | 0.199 | 1.068 | 1.372 | 1.161 |
| A_52_P636559 | AK053008; | 268469 | 9530033F24Rik | 0.506 | 0.53 | 0.567 | 1.485 | 1.451 | 1.363 |
| A_52_P425734 | NM_145146 | 280662 | Afm | 1.466 | 1.63 | 2.105 | 1.902 | 1.789 | 2.093 |
| A_52_P1011737 | AK039096; | 106258 | AI790442 | 0.224 | 0.44 | 0.438 | 1.367 | 1.308 | 1.503 |
| A_52_P537140 | AK047235; | | | 0.566 | 0.565 | 0.531 | 1.029 | 1.127 | 1.156 |
| A_51_P191990 | AK087548; | 26563 | Ror1 | 0.378 | 0.368 | 0.389 | 1.197 | 1.149 | 1.176 |
| A_51_P478930 | AK080016; | | | 0.64 | 0.623 | 0.816 | 1.097 | 1.039 | 1.17 |
| A_51_P252565 | NM_009660 | 11687 | Alox15 | 1.618 | 1.425 | 1.194 | 1.366 | 1.436 | 1.457 |
| A_52_P81252 | NM_172599 | 218978 | D14Ertd436e | 0.265 | 0.408 | 0.488 | 1.153 | 1.046 | 0.927 |
| A_51_P487004 | NM_027732 | 71241 | Dmrtc2 | 1.918 | 2.991 | 1.94 | 1.77 | 1.537 | 1.901 |
| A_51_P104162 | AK053379; | 328232 | | 0.351 | 0.468 | 0.481 | 1.289 | 1.159 | 1.082 |
| A_52_P555603 | AK032770; | 11787 | Apbb2 | 0.273 | 0.355 | 0.304 | 1.021 | 1.021 | 1.054 |
| A_52_P819356 | AK041684; | | | 0.651 | 0.704 | 0.763 | 1.654 | 1.657 | 1.821 |
| A_52_P1196751 | AK086488; | | | 0.314 | 0.63 | 0.521 | 1.285 | 1.098 | 1.263 |
| A_52_P277415 | AK077313; | 76252 | 0610006O14Rik | 0.0165 | 0.0277 | 0.0223 | 0.0497 | 0.043 | 0.0552 |
| A_52_P812362 | BC016205; | 408064 | BC064078 | 1.424 | 1.508 | 1.444 | 1.323 | 1.468 | 1.262 |
| A_51_P352572 | NM_019701 | 56365 | Clcnkb | 1.441 | 1.565 | 1.422 | 1.707 | 1.362 | 1.314 |
| A_52_P100042 | AK045143; | 320938 | Tnpo3 | 0.301 | 0.507 | 0.406 | 1.444 | 1.417 | 1.439 |
| A_51_P103397 | AK078521; | 54561 | Nap1l3 | 0.942 | 0.996 | 1.004 | 1.047 | 0.978 | 1.076 |
| A_52_P296244 | BC048734; | | | 2.333 | 2.799 | 1.851 | 2.371 | 2.371 | 2.372 |
| A_51_P305019 | AK020601; | 78423 | 9530049O05Rik | 0.201 | 0.4 | 0.429 | 1.187 | 1.059 | 1.249 |
| A_52_P3304 | NM_080446 | 117599 | Helb | 1.004 | 1.239 | 1.059 | 1.614 | 1.432 | 1.418 |
| A_51_P147651 | AK041797; XM_146705 | 245902 | | 0.511 | 0.729 | 0.963 | 1.47 | 1.214 | 1.502 |
| A_51_P203955 | NM_010260 | 14469 | Gbp2 | 2.194 | 2.061 | 2.214 | 2.45 | 2.681 | 2.345 |
| A_51_P176474 | AK010466; | 76484 | 2410012C07Rik | 0.842 | 0.903 | 1.004 | 0.996 | 1.036 | 1.127 |
| A_52_P165497 | NM_011385 | 20481 | Ski | 0.541 | 0.665 | 0.649 | 1.004 | 0.817 | 0.972 |
| A_51_P358714 | AK031433; | | | 0.23 | 0.294 | 0.418 | 1.005 | 0.775 | 1.214 |
| A_51_P106918 | NM_009051 | 19715 | Rex2 | 1.032 | 1.019 | 0.654 | 1.31 | 1.285 | 1.541 |
| A_51_P124315 | NM_177649 | 227712 | BC034076 | 0.654 | 0.611 | 0.726 | 1.267 | 1.174 | 1.278 |
| A_52_P183368 | NM_011535 | 21386 | Tbx3 | 0.707 | 0.655 | 0.649 | 0.948 | 0.84 | 0.841 |
| A_51_P140751 | NM_011397 | 20522 | Slc23a1 | 0.522 | 0.618 | 0.556 | 1.152 | 1.255 | 1.186 |
| A_51_P103406 | NM_011708 | 22371 | Vwf | 0.798 | 1.058 | 1.093 | 1.007 | 0.932 | 1.019 |
| A_51_P381069 | XM_132045 | 231252 | Chrna9 | 0.803 | 0.789 | 0.615 | 1.017 | 1.052 | 0.983 |
| A_52_P75971 | NM_026599 | 68178 | Cgnl1 | 0.432 | 0.439 | 0.529 | 0.801 | 0.761 | 0.771 |
| A_52_P135873 | XM_132015; | 231128 | BC037112 | 0.389 | 0.408 | 0.392 | 1.158 | 1.118 | 1.166 |
| | XM_488584 | | | | | | | | |
| A_51_P165082 | AB041601; XM_489523 | 224019 | D16Bwg1494e | 1.656 | 1.924 | 1.93 | 1.376 | 1.394 | 1.295 |
| A_51_P294566 | AK020687; | 78083 | 9930116N10Rik | 0.689 | 1.237 | 0.848 | 1.59 | 1.475 | 1.545 |
| A_51_P378051 | NM_009675 | 11754 | Aoc3 | 1.41 | 1.106 | 1.668 | 0.9 | 0.88 | 1.25 |
| A_52_P420608 | NM_201366 | 381371 | Gm1631 | 1.252 | 0.926 | 0.759 | 1.014 | 1.175 | 1.159 |
| A_51_P221291 | NM_009229 | 20650 | Sntb2 | 0.614 | 0.612 | 0.631 | 1.259 | 1.1 | 1.157 |
| A_52_P362805 | AK044730; | 269254 | Als4 | 0.692 | 0.79 | 0.89 | 1.216 | 1.11 | 1.17 |
| A_51_P109649 | AK018142; | 17263 | Gtl2 | 0.11 | 0.17 | 0.136 | 0.167 | 0.167 | 0.195 |
| A_52_P413348 | XM_127674 | 75698 | 3110001K24Rik | 0.593 | 0.709 | 0.685 | 1.434 | 1.352 | 1.356 |
| A_52_P400425 | NM_178656 | 193003 | A530088H08Rik | 0.241 | 0.469 | 0.547 | 0.79 | 0.835 | 1.024 |
| A_52_P147870 | NM_029963 | 77721 | Mrps5 | 0.574 | 0.622 | 0.64 | 1.321 | 1.377 | 1.297 |
| A_52_P57078 | AK052455; | | | 0.552 | 0.439 | 0.686 | 1.676 | 1.452 | 1.294 |
| A_52_P389874 | NM_011918 | 24131 | Ldb3 | 0.702 | 0.892 | 0.937 | 1.166 | 1.063 | 1.078 |
| A_52_P224125 | XM_622260 | | | 0.593 | 0.637 | 0.657 | 1.232 | 0.927 | 0.967 |
| A_52_P238468 | XM_133238 | 381853 | Gm160 | 0.673 | 0.817 | 0.882 | 1.027 | 1.116 | 1.106 |
| A_51_P253053 | XM_618867 | | | 0.601 | 1.748 | 0.724 | 1.594 | 1.644 | 2.037 |
| A_52_P500979 | NM_013628 | 18548 | Pcsk1 | 4.338 | 3.184 | 4.164 | 4.966 | 5.086 | 4.923 |
| A_51_P207934 | NM_199016 | 224794 | Enpp4 | 0.71 | 0.576 | 0.811 | 1.068 | 0.833 | 1.143 |
| A_52_P423384 | BC057551; XM_354546 | 104248 | Cabin1 | 0.481 | 0.424 | 0.586 | 1.189 | 1.05 | 1.408 |
| A_52_P560006 | NM_008426 | 16519 | Kcnj3 | 2.321 | 2.818 | 2.608 | 2.191 | 1.875 | 2.19 |
| A_52_P654624 | AK005333; | 236604 | 4933439C20Rik | 0.543 | 0.696 | 0.523 | 1.316 | 1.231 | 1.139 |
| A_52_P32502 | AK020822; | 77944 | A930007B11Rik | 1.212 | 1.019 | 1.21 | 2.052 | 1.291 | 1.824 |
| A_51_P302377 | NM_008009 | 14181 | Fgfbp1 | 1.542 | 1.782 | 1.567 | 1.002 | 1.052 | 1.132 |
| A_52_P255224 | AK032711; XM_486482 | 234371 | BC021367 | 0.812 | 0.712 | 0.548 | 2.288 | 1.957 | 2.287 |
| A_52_P571746 | XM_355533; | 230863 | LOC381571 | 1.076 | 1.21 | 1.362 | 2.199 | 2.23 | 2.231 |
| | XM_357399 | | | | | | | | |
| A_52_P458916 | AK016751; | 218850 | D14Abb1e | 0.488 | 0.694 | 0.807 | 1.15 | 1.185 | 1.257 |
| A_52_P286970 | XM_131162; | 76561 | Snx7 | 1.591 | 2.536 | 1.327 | 1.128 | 1.271 | 0.96 |
| | XM_127565 | | | | | | | | |
| A_51_P266644 | BC055753; XM_125538 | 140742 | | 1.51 | 1.548 | 1.621 | 1.064 | 0.96 | 1.069 |
| A_52_P233673 | NM_020594 | 57432 | Zc3hdc8 | 0.731 | 0.498 | 0.877 | 1.204 | 1.041 | 1.395 |
| A_52_P1084149 | AK051413; | 553138 | 9930021J03Rik | 0.447 | 0.508 | 0.452 | 1.499 | 1.58 | 1.485 |
| A_52_P413661 | BC079861; XM_484162 | 217716 | Mlh3 | 0.513 | 0.589 | 0.64 | 1.73 | 1.608 | 1.611 |
| A_51_P404193 | NM_022435 | 64406 | Sp5 | 1.798 | 1.335 | 1.388 | 3.365 | 3.261 | 3.306 |
| A_52_P565940 | AF419220; | 18193 | Nsd1 | 0.487 | 0.593 | 0.616 | 1.236 | 1.181 | 1.08 |
| A_51_P514177 | NM_028838 | 74249 | Lrrc2 | 0.911 | 0.956 | 0.975 | 1.199 | 1.209 | 1.304 |
| A_52_P237357 | AF095690; | | | 0.454 | 0.422 | 0.442 | 1.34 | 1.372 | 1.159 |
| A_51_P240811 | NM_009290 | 20890 | Wnt8a | 4.437 | 4.432 | 3.479 | 4.373 | 5.655 | 4.468 |
| A_51_P236267 | NM_009183 | 20452 | St8sia4 | 1.305 | 1.382 | 1.289 | 1.168 | 1.234 | 1.322 |
| A_52_P405340 | U76762; | 14158 | Fert2 | 0.434 | 0.372 | 0.598 | 1.654 | 1.494 | 1.493 |
| A_52_P661144 | XM_485365 | | | 0.511 | 0.546 | 0.638 | 1.627 | 1.676 | 2.009 |
| A_52_P1044262 | AK051949; | | | 0.374 | 0.535 | 0.443 | 1.042 | 1.044 | 1.091 |
| A_52_P553100 | AK040413; | | | 0.696 | 0.886 | 0.727 | 2.086 | 2.022 | 1.86 |
| A_51_P358344 | NM_007732 | 12821 | Col17a1 | 0.283 | 0.661 | 0.876 | 1.118 | 0.984 | 1.246 |
| A_51_P321579 | XM_131865; | 433809 | LOC433809 | 0.495 | 0.534 | 0.598 | 1.155 | 1.087 | 1.237 |
| | XM_196334 | | | | | | | | |
| A_52_P485939 | NM_026228 | 67547 | Slc39a8 | 3.203 | 2.606 | 3.687 | 2.136 | 1.903 | 2.146 |
| A_52_P199922 | | | | 0.316 | 0.331 | 0.255 | 1.479 | 1.195 | 1.071 |
| A_51_P463791 | BC051947; | 58212 | | 0.648 | 0.879 | 1.013 | 1.447 | 1.472 | 1.498 |
| A_52_P487362 | NM_028980 | 74521 | 8430415E04Rik | 3.136 | 2.67 | 3.124 | 4.295 | 4.001 | 3.546 |
| A_51_P341137 | AK035176; | 224829 | Trerf1 | 0.563 | 0.731 | 0.649 | 1.51 | 1.436 | 1.382 |
| A_51_P407323 | NM_007976 | 14067 | F5 | 0.837 | 0.684 | 0.686 | 2.154 | 2.023 | 1.925 |
| A_51_P272363 | NM_134160 | 171166 | Mcoln3 | 2.423 | 3.074 | 2.25 | 1.242 | 1.432 | 0.843 |
| A_52_P430927 | AK042385; | 11908 | Atf1 | 0.409 | 0.436 | 0.428 | 1.263 | 1.186 | 1.26 |
| A_52_P626069 | AK040994; XM_284439 | 109151 | Chd9 | 0.449 | 0.512 | 0.687 | 1.302 | 1.102 | 1.65 |
| A_52_P355276 | NM_001002764 | 103677 | AI317223 | 0.704 | 0.605 | 0.784 | 1.531 | 1.432 | 1.606 |
| A_52_P650215 | NM_029278 | 75416 | 2610033H07Rik | 0.869 | 0.561 | 1.153 | 1.294 | 1.106 | 1.919 |
| A_52_P416123 | AK020480; | 77358 | 9430069I07Rik | 0.584 | 0.644 | 1.001 | 1.681 | 1.367 | 1.728 |
| A_51_P189631 | NM_172427 | 76455 | 2310067E19Rik | 0.318 | 0.341 | 0.373 | 1.015 | 0.905 | 0.892 |
| A_52_P16973 | AK081934; | 320391 | D930017J03Rik | 0.56 | 0.604 | 0.612 | 1.894 | 1.918 | 1.856 |
| A_52_P947879 | AK048751; | | | 0.354 | 0.496 | 0.449 | 1.501 | 1.634 | 1.919 |
| A_52_P538515 | BC049181; | 226412 | R3hdm | 0.209 | 0.659 | 0.565 | 1.913 | 1.725 | 1 |
| A_52_P179250 | AK129127; | 230249 | AI314180 | 0.641 | 0.606 | 0.56 | 3.467 | 4.289 | 6.052 |
| A_52_P795628 | AK086352; | | | 0.551 | 1.049 | 0.818 | 3.259 | 4.511 | 4.2 |
| A_51_P144712 | NM_178930 | 107338 | Gbf1 | 0.424 | 0.476 | 0.425 | 1.192 | 1.19 | 1.292 |
| A_52_P655008 | AK044382; | 320678 | A930037G23Rik | 0.364 | 0.363 | 0.425 | 1.339 | 1.302 | 1.272 |
| A_52_P577072 | NM_011901 | 24074 | Taf7 | 0.654 | 0.591 | 0.759 | 1.655 | 1.407 | 1.87 |
| A_52_P636199 | NM_008017 | 14211 | Smc2l1 | 0.482 | 0.452 | 0.534 | 1.172 | 1.125 | 1.415 |
| A_51_P447189 | AK041326; XM_358090 | 385154 | | 0.755 | 0.808 | 0.675 | 2.409 | 2.277 | 2.233 |
| A_52_P1180315 | AK049088; | 74022 | 3930401K13Rik | 0.255 | 0.505 | 0.516 | 1.337 | 1.179 | 0.966 |
| A_52_P412167 | NM_080467 | 140494 | Atp6v0a4 | 0.553 | 1.045 | 0.878 | 1.777 | 2.017 | 2.418 |
| A_52_P335089 | BE198552; | | | 0.338 | 0.471 | 0.43 | 0.921 | 0.981 | 0.901 |
| A_52_P6621 | | | | 0.661 | 0.506 | 1.607 | 1.455 | 1.079 | 2.626 |
| A_51_P343129 | AK012079; | 70472 | Atad2 | 0.633 | 0.896 | 0.848 | 1.965 | 1.637 | 1.677 |
| A_51_P144417 | NM_181750 | 226412 | R3hdm | 0.745 | 0.757 | 0.789 | 2.029 | 1.993 | 2.191 |
| A_52_P350477 | NM_139295 | 193813 | Mcfd2 | 1.118 | 1.377 | 1.329 | 0.667 | 0.758 | 0.786 |
| A_51_P418884 | NM_001013789 | 432823 | | 0.496 | 0.536 | 0.695 | 1.716 | 1.402 | 1.593 |
| A_52_P581138 | NM_212450 | 329506 | D2Ertd485e | 0.457 | 0.451 | 0.472 | 1.47 | 1.244 | 1.239 |
| A_52_P434549 | NM_023190 | 56215 | Acin1 | 0.489 | 0.399 | 0.44 | 1.376 | 1.207 | 1.143 |
| A_52_P384392 | AK080827; | 328087 | D930001B02 | 0.533 | 0.516 | 0.471 | 1.258 | 1.062 | 1.099 |
| A_51_P270725 | AK089493; | 102093 | Phkb | 0.756 | 0.923 | 0.755 | 2.239 | 1.725 | 2.287 |
| A_51_P193019 | NM_011157 | 19073 | Prg1 | 0.263 | 0.419 | 0.589 | 1.602 | 0.893 | 0.295 |
| A_52_P1172548 | AK084146; | 13204 | Dhx15 | 0.655 | 0.839 | 0.889 | 2.469 | 2.249 | 2.786 |
| A_51_P114606 | NM_016965 | 50884 | Nckap1 | 1.512 | 1.408 | 1.677 | 0.736 | 0.694 | 0.805 |
| A_52_P306387 | NM_178709 | 235315 | D130054N24Rik | 0.574 | 0.504 | 0.652 | 1.816 | 1.515 | 1.965 |
| A_52_P287576 | AK036308; | | | 0.284 | 0.537 | 0.429 | 1.515 | 1.516 | 1.834 |
| A_52_P501767 | AK012272; | 20637 | Snrp70 | 0.263 | 0.243 | 0.263 | 1.047 | 1 | 0.926 |
| A_52_P995717 | AK045222; | | | 0.672 | 0.74 | 0.709 | 2.392 | 1.854 | 2.412 |

TABLE 3A
200 Gene Subset (Germline Competent)

| | | | | Clone 1 | | | Clone 2 | | |
|---|---|---|---|---|---|---|---|---|---|
| Agilent Probe ID | Genbank | Entrez ID | Gene Symbol | rep. 1 | rep. 2 | rep. 3 | rep. 1 | rep. 2 | rep. 3 |
| A_51_P237040 | NM_008711 | 18121 | Nog | 0.266 | 0.299 | 0.281 | 0.396 | 0.368 | 0.407 |
| A_51_P335460 | NM_009132 | 20259 | Scin | 0.718 | 0.631 | 0.719 | 0.678 | 0.674 | 0.666 |
| A_52_P391505 | NM_144513 | 17263 | Gtl2 | 1.36 | 1.291 | 1.327 | 1.053 | 1.04 | 0.852 |
| A_51_P497100 | NM_010706 | 16855 | Lgals4 | 2.115 | 2.049 | 2.368 | 1.716 | 1.861 | 1.527 |
| A_51_P378371 | | | | 0.454 | 0.542 | 0.39 | 0.468 | 0.484 | 0.468 |
| A_51_P298266 | NM_025681 | 66643 | Lix1 | 0.507 | 0.414 | 0.416 | 0.554 | 0.584 | 0.54 |
| A_52_P269158 | NM_001003948 | 98496 | AL024069 | 0.316 | 0.33 | 0.33 | 0.388 | 0.393 | 0.443 |
| A_51_P311038 | BC009666; XM_134720 | 57267 | Apba3 | 0.334 | 0.341 | 0.339 | 0.446 | 0.472 | 0.482 |
| A_51_P457187 | NM_028075 | 72049 | Tnfrsf13c | 2.82 | 3.275 | 3.026 | 2.654 | 3.432 | 2.546 |
| A_52_P68702 | NM_145148 | 232288 | Frmd4b | 0.545 | 0.476 | 0.538 | 0.501 | 0.43 | 0.424 |
| A_52_P293443 | AK087208; | 13819 | | 1.278 | 1.075 | 1.13 | 1.496 | 1.604 | 1.349 |
| A_51_P157986 | NM_029930 | 77574 | 3321401G04Rik | 0.426 | 0.339 | 0.345 | 0.368 | 0.318 | 0.37 |
| A_52_P1013441 | | | | 3.248 | 3.164 | 3.191 | 4.558 | 5.61 | 4.683 |
| A_51_P418375 | NM_023844 | 67374 | Jam2 | 1.691 | 1.467 | 1.625 | 2.142 | 2.132 | 2.288 |
| A_52_P557940 | AK004302; | 66704 | 4921506I22Rik | 2.114 | 1.863 | 2.221 | 2.17 | 2.013 | 2.051 |
| A_51_P480311 | NM_010168 | 14061 | F2 | 1.991 | 1.809 | 1.969 | 2.465 | 2.4 | 1.874 |
| A_52_P200458 | NM_010242 | 14345 | Fut4 | 0.429 | 0.343 | 0.355 | 0.453 | 0.391 | 0.512 |
| A_51_P463846 | NM_145545 | 229900 | 9830147J24Rik | 0.694 | 0.519 | 0.59 | 0.86 | 0.816 | 0.817 |
| A_51_P162474 | AF285577; | 107815 | Scml2 | 0.539 | 0.413 | 0.405 | 0.643 | 0.645 | 0.71 |
| A_51_P165244 | NM_018734 | 55932 | Gbp4 | 0.585 | 0.512 | 0.474 | 1.008 | 0.981 | 1.006 |
| A_52_P59318 | NM_133832 | 98711 | Rdh10 | 0.306 | 0.273 | 0.275 | 0.373 | 0.395 | 0.445 |
| A_52_P438036 | NM_175271 | 78134 | Gpr23 | 0.376 | 0.334 | 0.36 | 0.455 | 0.441 | 0.489 |
| A_52_P474242 | BE457768; | | | 0.499 | 0.53 | 0.46 | 0.69 | 0.658 | 0.726 |
| A_51_P499854 | NM_010284 | 14600 | Ghr | 0.407 | 0.375 | 0.412 | 0.413 | 0.429 | 0.489 |
| A_51_P455647 | NM_009801 | 12349 | Car2 | 0.335 | 0.288 | 0.301 | 0.568 | 0.516 | 0.638 |
| A_52_P103929 | NM_009306 | 20979 | Syt1 | 0.382 | 0.356 | 0.39 | 0.419 | 0.498 | 0.423 |
| A_51_P149469 | NM_009608 | 11464 | Actc1 | 0.324 | 0.387 | 0.384 | 0.496 | 0.551 | 0.471 |
| A_52_P557293 | NM_015775 | 50528 | Tmprss2 | 0.277 | 0.205 | 0.179 | 0.45 | 0.457 | 0.355 |
| A_52_P50496 | AY048587; XM_619453 | 114640 | Tifp39 | 0.454 | 0.501 | 0.422 | 0.661 | 0.638 | 0.694 |
| A_51_P180413 | NM_176930 | 319504 | C130076O07Rik | 0.359 | 0.393 | 0.415 | 0.471 | 0.514 | 0.494 |
| A_51_P229613 | NM_008792 | 18549 | Pcsk2 | 0.512 | 0.483 | 0.515 | 0.58 | 0.58 | 0.504 |
| A_51_P121635 | NM_030700 | 80884 | Maged2 | 0.378 | 0.371 | 0.409 | 0.492 | 0.475 | 0.481 |
| A_51_P481777 | NM_018756 | 54382 | Tcstv1 | 1.532 | 1.822 | 1.792 | 2.619 | 3.155 | 2.956 |
| A_51_P412817 | NM_177617 | 216851 | D330014H01Rik | 3.151 | 3.039 | 3.326 | 2.136 | 2.194 | 1.865 |
| A_51_P387528 | AK019664; | 78801 | Ak7 | 2.693 | 2.814 | 2.57 | 3.786 | 3.834 | 3.71 |
| A_52_P78168 | AK031518; | | | 2.108 | 1.677 | 1.617 | 1.783 | 1.791 | 1.711 |
| A_52_P274960 | NM_001005508 | 226652 | 6030405P05Rik | 1.83 | 1.82 | 1.948 | 2.07 | 2.181 | 2.076 |
| A_52_P1188270 | AK042362; | | | 3.076 | 1.828 | 1.917 | 2.932 | 3.046 | 2.87 |
| A_51_P291227 | NM_001005508 | 226652 | 6030405P05Rik | 1.876 | 1.826 | 1.86 | 2.054 | 2.185 | 2.004 |
| A_52_P377299 | AK048360; | 271377 | Zbtb11 | 1.927 | 1.892 | 1.923 | 1.678 | 1.79 | 1.546 |
| A_51_P297131 | NM_009502 | 22330 | Vcl | 0.402 | 0.372 | 0.378 | 0.411 | 0.377 | 0.401 |
| A_52_P804224 | | | | 1.522 | 1.688 | 1.828 | 2.31 | 2.487 | 2.241 |
| A_51_P442894 | AK048310; | 621603 | C130048D07Rik | 2.632 | 2.364 | 2.281 | 3.657 | 3.714 | 3.102 |
| A_52_P309718 | AK173099; | 218850 | D14Abb1e | 2.324 | 2.579 | 2.18 | 2.614 | 2.72 | 2.711 |
| A_52_P359010 | AK129478; | 72193 | Sfrs2ip | 2.152 | 2.149 | 2.122 | 2.349 | 2.38 | 2.318 |
| A_51_P410813 | AK079732; | | | 2.732 | 2.716 | 2.559 | 2.898 | 2.605 | 2.3 |
| A_52_P382565 | XM_483943 | 432493 | LOC432493 | 0.976 | 1.222 | 1.221 | 1.492 | 1.439 | 1.198 |
| A_51_P333253 | NM_178440 | 246177 | Myo1g | 1.419 | 1.413 | 1.621 | 1.544 | 1.504 | 1.394 |
| A_52_P113190 | NM_053214 | 17916 | Myo1f | 1.428 | 1.513 | 1.396 | 1.988 | 1.959 | 2.083 |
| A_51_P473953 | XM_358600; | 381498 | 4930592C13Rik | 0.42 | 0.306 | 0.298 | 0.365 | 0.365 | 0.38 |
| | XM_355454 | | | | | | | | |
| A_52_P519870 | AB010340; | | | 2.391 | 3.34 | 2.621 | 3.76 | 3.72 | 1.955 |
| A_51_P266403 | AK009163; | 76730 | 2310005C01Rik | 3.079 | 3.053 | 3.092 | 2.733 | 2.967 | 2.734 |
| A_52_P3698 | NM_001015099 | 217558 | | 1.885 | 1.84 | 1.762 | 2.392 | 2.296 | 2.486 |
| A_52_P581100 | NM_027285 | 70005 | 1700029I01Rik | 2.295 | 3.266 | 2.708 | 3.168 | 3.146 | 2.879 |
| A_51_P276305 | NM_009292 | 20899 | Stra8 | 3.137 | 3.069 | 3.174 | 2.824 | 2.972 | 2.777 |
| A_51_P455997 | Y13832; | 17263 | Gtl2 | 1.505 | 1.455 | 1.44 | 0.998 | 1.082 | 0.998 |
| A_52_P656336 | NM_023733 | 74114 | Crot | 0.407 | 0.351 | 0.381 | 0.755 | 0.647 | 0.8 |
| A_51_P512172 | AK010953; XM_125673 | 52463 | Cxxc6 | 2.864 | 2.384 | 2.216 | 2.184 | 2.11 | 2.068 |
| A_51_P282227 | NM_020577 | 57344 | As3mt | 2.651 | 2.607 | 2.876 | 3.224 | 3.327 | 3.147 |
| A_52_P386544 | NM_026594 | 68172 | 4930517K11Rik | 2.843 | 2.618 | 2.802 | 2.673 | 2.913 | 2.894 |
| A_51_P125679 | AK010725; XM_134948 | 73673 | 2410076I21Rik | 2.867 | 2.332 | 2.464 | 3.518 | 3.661 | 3.727 |
| A_51_P164504 | NM_007469 | 11812 | Apoc1 | 3.245 | 2.908 | 3.052 | 2.863 | 3.059 | 2.591 |
| A_52_P240706 | NM_007543 | 26367 | Ceacam2 | 2.285 | 1.866 | 1.892 | 2.654 | 2.556 | 2.628 |
| A_52_P420369 | NM_029444 | 75801 | 4930447C04Rik | 3.256 | 2.433 | 2.971 | 2.042 | 2.189 | 2.206 |
| A_51_P242399 | NM_031170 | 16691 | Krt2-8 | 0.193 | 0.233 | 0.208 | 0.403 | 0.4 | 0.39 |
| A_51_P158037 | NM_013505 | 13506 | Dsc2 | 0.271 | 0.249 | 0.258 | 0.437 | 0.415 | 0.453 |
| A_52_P258116 | NM_009521 | 22415 | Wnt3 | 0.156 | 0.164 | 0.16 | 0.312 | 0.309 | 0.296 |
| A_52_P286166 | NM_001002927 | 18619 | Penk1 | 0.497 | 0.324 | 0.339 | 0.671 | 0.658 | 0.647 |
| A_52_P399095 | NM_177793 | 327747 | 9030224M15Rik | 0.366 | 0.325 | 0.163 | 0.433 | 0.401 | 0.466 |
| A_52_P470663 | | | | 0.377 | 0.334 | 0.403 | 0.467 | 0.439 | 0.566 |
| A_52_P376169 | NM_177139 | 320343 | E130115E03Rik | 0.314 | 0.285 | 0.295 | 0.539 | 0.508 | 0.528 |
| A_51_P512085 | NM_130449 | 140792 | Colec12 | 0.335 | 0.364 | 0.296 | 0.382 | 0.368 | 0.385 |
| A_52_P676403 | NM_019494 | 56066 | Cxcl11 | 0.525 | 0.596 | 0.597 | 0.761 | 0.809 | 0.704 |
| A_51_P354307 | NM_172469 | 209195 | Clic6 | 0.43 | 0.319 | 0.329 | 0.398 | 0.398 | 0.387 |
| A_52_P569375 | NM_010203 | 14176 | Fgf5 | 0.776 | 0.678 | 0.554 | 0.45 | 0.467 | 0.574 |
| A_51_P162481 | NM_133194 | 107815 | Scml2 | 0.432 | 0.357 | 0.385 | 0.594 | 0.55 | 0.618 |
| A_51_P202911 | NM_144882 | 67198 | 2810022L02Rik | 0.297 | 0.283 | 0.328 | 0.346 | 0.333 | 0.365 |
| A_51_P418029 | NM_175541 | 245631 | Mum1l1 | 0.319 | 0.265 | 0.278 | 0.419 | 0.397 | 0.534 |
| A_52_P93837 | NM_008604 | 17380 | Mme | 0.559 | 0.439 | 0.513 | 0.653 | 0.573 | 0.747 |
| A_51_P275435 | NM_172411 | 71874 | 2310007B03Rik | 0.368 | 0.492 | 0.206 | 0.875 | 0.83 | 0.857 |
| A_51_P199888 | NM_026082 | 67299 | Dock7 | 0.407 | 0.403 | 0.245 | 0.558 | 0.428 | 0.546 |
| A_52_P211956 | NM_025658 | 66607 | Ms4a4d | 0.61 | 0.455 | 0.457 | 0.534 | 0.519 | 0.685 |
| A_51_P485985 | NM_134163 | 171170 | Mbnl3 | 0.223 | 0.181 | 0.161 | 0.625 | 0.543 | 0.697 |
| A_52_P630964 | NM_027533 | 70747 | Tspan2 | 0.361 | 0.283 | 0.324 | 0.499 | 0.473 | 0.525 |
| A_51_P294505 | NM_025658 | 66607 | Ms4a4d | 0.63 | 0.414 | 0.365 | 0.532 | 0.521 | 0.629 |
| A_52_P82488 | NM_021476 | 58861 | Cysltr1 | 0.319 | 0.379 | 0.306 | 0.5 | 0.438 | 0.534 |
| A_52_P634829 | NM_023835 | 76681 | Trim12 | 0.59 | 0.561 | 0.672 | 1.012 | 0.996 | 0.866 |
| A_52_P460882 | XM_130319; | 73668; 14725 | Ttc21b | 0.412 | 0.637 | 0.533 | 0.396 | 0.633 | 0.502 |
| | XM_130308 | | | | | | | | |
| A_52_P197826 | NM_010397 | 15039 | H2-T22 | 0.541 | 0.505 | 0.41 | 0.653 | 0.634 | 0.622 |
| A_51_P240253 | NM_019662 | 56437 | Rrad | 0.316 | 0.322 | 0.314 | 0.392 | 0.337 | 0.342 |
| A_51_P101975 | NM_010205 | 14179 | Fgf8 | 0.412 | 0.39 | 0.413 | 0.415 | 0.454 | 0.385 |
| A_52_P21595 | NM_028238 | 72433 | Rab38 | 0.234 | 0.188 | 0.168 | 0.439 | 0.467 | 0.472 |
| A_52_P402786 | NM_008935 | 19126 | Prom1 | 0.37 | 0.396 | 0.335 | 0.551 | 0.506 | 0.594 |
| A_52_P126158 | NM_008326 | 15944 | Ifi1 | 0.237 | 0.208 | 0.22 | 0.415 | 0.412 | 0.425 |
| A_52_P676510 | NM_011579 | 21822 | Tgtp | 0.225 | 0.162 | 0.155 | 0.382 | 0.378 | 0.362 |
| A_52_P175685 | NM_008390 | 16362 | Irf1 | 0.401 | 0.37 | 0.3 | 0.663 | 0.637 | 0.749 |
| A_52_P384718 | BC052056; XM_283264 | 320873 | | 0.508 | 0.441 | 0.458 | 0.541 | 0.512 | 0.578 |
| A_52_P176333 | NM_001005787 | 12695 | Cipp | 0.372 | 0.357 | 0.365 | 0.452 | 0.392 | 0.42 |
| A_52_P358951 | AK081999; XM_134736 | 271457; 22688 | Rab5a | 0.417 | 0.378 | 0.448 | 0.464 | 0.449 | 0.46 |
| A_51_P168646 | AK039580; | | | 0.442 | 0.434 | 0.509 | 0.537 | 0.548 | 0.598 |
| A_52_P620345 | NM_133979 | 102566 | AI604368 | 0.368 | 0.402 | 0.382 | 0.441 | 0.438 | 0.493 |
| A_52_P253179 | AK077477; | 16009 | Igfbp3 | 0.397 | 0.511 | 0.257 | 0.517 | 0.468 | 0.482 |
| A_51_P397003 | XM_133990 | 68904 | 1110065L07Rik | 0.45 | 0.438 | 0.438 | 0.542 | 0.479 | 0.613 |
| A_52_P74441 | NM_011570 | 21753 | Tes | 0.423 | 0.418 | 0.328 | 0.54 | 0.558 | 0.659 |
| A_52_P255250 | NM_009543 | 22644 | Rnf103 | 0.532 | 0.371 | 0.413 | 0.471 | 0.455 | 0.491 |
| A_52_P575505 | NM_008994 | 19302 | Pxmp3 | 0.305 | 0.306 | 0.317 | 0.463 | 0.445 | 0.525 |
| A_52_P469789 | NM_007588 | 12311 | Calcr | 0.48 | 0.383 | 0.445 | 0.625 | 0.707 | 0.696 |
| A_51_P345422 | NM_010747 | 17096 | Lyn | 0.244 | 0.247 | 0.256 | 0.373 | 0.392 | 0.424 |
| A_51_P356003 | NM_001007580 | 333564 | Gm784 | 0.494 | 0.445 | 0.513 | 0.509 | 0.483 | 0.534 |
| A_52_P680941 | NM_010284 | 14600 | Ghr | 0.389 | 0.396 | 0.371 | 0.448 | 0.445 | 0.488 |
| A_52_P380263 | NM_013723 | 27205 | Podxl | 0.395 | 0.422 | 0.286 | 0.532 | 0.511 | 0.504 |
| A_52_P286528 | BC055771; | 102954 | Nudt11 | 0.411 | 0.273 | 0.263 | 0.433 | 0.371 | 0.517 |
| A_51_P105068 | NM_027990 | 71897 | 2310010M24Rik | 0.459 | 0.371 | 0.462 | 0.48 | 0.484 | 0.498 |
| A_52_P132608 | NM_172685 | 229731 | Slc25a24 | 0.358 | 0.291 | 0.25 | 0.504 | 0.401 | 0.432 |
| A_51_P467505 | NM_013689 | 21682 | Tec | 0.28 | 0.242 | 0.227 | 0.502 | 0.464 | 0.499 |
| A_52_P153685 | AK129323; | 70422 | 2810417D08Rik | 0.452 | 0.397 | 0.407 | 0.587 | 0.526 | 0.735 |
| A_51_P174943 | NM_008485 | 16782 | Lamc2 | 0.234 | 0.255 | 0.286 | 0.433 | 0.411 | 0.451 |
| A_52_P301821 | NM_053078 | 27528 | D0H4S114 | 0.442 | 0.427 | 0.469 | 0.472 | 0.525 | 0.486 |
| A_52_P532910 | NM_024427 | 22003 | Tpm1 | 0.358 | 0.392 | 0.35 | 0.386 | 0.39 | 0.433 |
| A_51_P521084 | NM_025626 | 66540 | 3110001A13Rik | 0.408 | 0.406 | 0.391 | 0.474 | 0.456 | 0.494 |
| A_52_P680436 | NM_025793 | 66840 | Wdr45l | 0.344 | 0.369 | 0.331 | 0.651 | 0.557 | 0.675 |
| A_51_P164219 | NM_011909 | 24110 | Usp18 | 0.435 | 0.307 | 0.303 | 0.435 | 0.448 | 0.457 |
| A_52_P421234 | NM_133832 | 98711 | Rdh10 | 0.356 | 0.309 | 0.338 | 0.406 | 0.4 | 0.427 |
| A_52_P586157 | NM_022309 | 12400 | Cbfb | 0.481 | 0.354 | 0.384 | 0.606 | 0.523 | 0.75 |
| A_51_P262171 | NM_008326 | 15944 | Ifi1 | 0.29 | 0.264 | 0.261 | 0.474 | 0.475 | 0.507 |
| A_52_P452689 | NM_007498 | 11910 | Atf3 | 0.291 | 0.326 | 0.293 | 0.307 | 0.321 | 0.307 |
| A_52_P295933 | AK045060; | 14051 | B130023L16Rik | 0.336 | 0.293 | 0.594 | 0.466 | 0.5 | 0.466 |
| A_51_P222773 | NM_010446 | 15376 | Foxa2 | 0.423 | 0.314 | 0.4 | 0.447 | 0.809 | 0.489 |
| A_52_P361534 | NM_009521 | 22415 | Wnt3 | 0.0992 | 0.0782 | 0.0774 | 0.211 | 0.198 | 0.243 |
| A_51_P507880 | NM_028980 | 74521 | 8430415E04Rik | 0.223 | 0.221 | 0.235 | 0.35 | 0.326 | 0.332 |
| A_52_P574668 | NM_011851 | 23959 | Nt5e | 0.15 | 0.159 | 0.148 | 0.316 | 0.285 | 0.335 |
| A_52_P957500 | XM_622354 | | | 2.438 | 1.963 | 2.267 | 2.142 | 2.231 | 1.855 |
| A_52_P345626 | AY149175; | 386626 | Mzf6d | 2.677 | 3.059 | 3.002 | 3.203 | 2.748 | 3.757 |
| A_52_P84015 | AK037762; | 11863 | | 3.675 | 3.344 | 3.705 | 4.008 | 3.981 | 3.736 |
| A_52_P432464 | AK053863; | 192196 | Luc7l2 | 2.643 | 3.226 | 2.225 | 1.973 | 2.004 | 1.643 |
| A_52_P376502 | BC025011; | 227541 | Camk1d | 1.575 | 1.641 | 1.563 | 1.989 | 2.543 | 1.959 |
| A_52_P354785 | AK037929; | 14852 | Gspt1 | 2.114 | 1.965 | 1.835 | 1.904 | 1.907 | 2.055 |
| A_51_P398766 | NM_010259 | 14468 | Gbp1 | 0.374 | 0.372 | 0.361 | 0.894 | 0.876 | 0.949 |
| A_52_P486234 | AK033497; | 22680 | | 3.81 | 2.878 | 2.669 | 3.19 | 3.59 | 3.068 |
| A_52_P628212 | AK044339; | 26380 | | 9.353 | 5.604 | 7.112 | 6.273 | 6.769 | 7.237 |
| A_52_P445799 | AK046777; | 52490 | D18Ertd289e | 0.676 | 0.602 | 0.389 | 0.613 | 0.532 | 0.592 |
| A_51_P268167 | BC037232; XM_621365 | 229363; 218850 | Gmps | 2.665 | 2.898 | 2.35 | 2.187 | 2.366 | 2.198 |
| A_51_P153079 | NM_133923 | 101100 | 4833441J24Rik | 1.312 | 1.174 | 1.271 | 1.222 | 1.343 | 1.299 |
| A_52_P584874 | NM_152895 | 75605 | Jarid1b | 2.228 | 2.064 | 2.215 | 1.844 | 1.912 | 1.769 |
| A_52_P307801 | NM_007847 | 13222 | Defcr-rs2 | 0.119 | 0.091 | 0.0705 | 0.133 | 0.175 | 0.108 |
| A_52_P163148 | AB010370; | 544945 | | 2.883 | 2.86 | 2.64 | 3.58 | 3.156 | 3.085 |
| A_51_P171728 | NM_011926 | 26365 | Ceacam1 | 2.703 | 1.992 | 2.013 | 3.11 | 3.457 | 2.977 |
| A_51_P141660 | AK053665; XM_358382 | 67776; 235380 | Loh11cr2a | 2.996 | 3.339 | 3.08 | 2.433 | 2.601 | 2.365 |
| A_52_P532323 | NM_178410 | 218518 | Marveld2 | 0.399 | 0.326 | 0.264 | 0.434 | 0.363 | 0.431 |
| A_51_P337125 | NM_010566 | 16331 | Inpp5d | 3.162 | 2.936 | 3.033 | 4.399 | 4.495 | 4.161 |
| A_52_P336259 | AK087367; XM_485576 | 319467 | A930002C04Rik | 1.736 | 2.368 | 2.023 | 1.677 | 1.679 | 1.401 |
| A_51_P318577 | AK010729; | 73671 | 2410078J06Rik | 3.283 | 4.227 | 3.815 | 6.599 | 6.454 | 5.366 |
| A_52_P324934 | AK045391; | 16403 | | 1.983 | 2.443 | 1.872 | 2.691 | 2.881 | 2.226 |
| A_52_P826666 | AK007019; | 51799 | Rap2ip | 4.063 | 3.142 | 3.547 | 3.365 | 3.764 | 3.267 |
| A_51_P200223 | XM_619497 | | | 2.332 | 2.259 | 2.383 | 1.575 | 1.797 | 1.553 |
| A_51_P228472 | NM_008343 | 16009 | Igfbp3 | 0.288 | 0.22 | 0.276 | 0.283 | 0.322 | 0.364 |
| A_52_P206573 | NM_027427 | 70439 | Taf15 | 2.14 | 3.003 | 2.282 | 1.904 | 2.123 | 1.956 |
| A_52_P354363 | NM_026574 | 68142 | | 1.74 | 1.737 | 1.937 | 1.525 | 1.61 | 1.378 |
| A_52_P651469 | NM_001012726 | 503692 | | 5.11 | 4.303 | 5.085 | 5.606 | 6.363 | 4.012 |
| A_51_P463789 | BC051947; | 58212 | | 3.253 | 2.961 | 3.321 | 2.093 | 2.306 | 1.934 |
| A_52_P18887 | NM_011468 | 20755 | Sprr2a | 0.21 | 0.171 | 0.214 | 0.282 | 0.277 | 0.25 |
| A_51_P223132 | NM_031183; | 83395; 70549 | Sp6 | 2.329 | 2.088 | 2.196 | 1.457 | 1.639 | 1.36 |
| | XM_486227 | | | | | | | | |
| A_52_P223083 | BC038323; | 72096 | 2010208K18Rik | 4.486 | 6.15 | 3.745 | 3.784 | 3.918 | 3.378 |
| A_52_P523399 | NM_029444 | 75801 | 4930447C04Rik | 3.312 | 7.019 | 4.341 | 3.597 | 4.009 | 2.819 |
| A_52_P282000 | AK020248; | 13800 | Enah | 2.7 | 2.435 | 2.683 | 2.632 | 2.812 | 2.59 |
| A_52_P152983 | AK011739; | 26886 | Cenph | 2.134 | 3.107 | 2.171 | 2.114 | 2.408 | 2.327 |
| A_52_P26416 | AF106279; | 16782 | | 0.254 | 0.252 | 0.241 | 0.358 | 0.391 | 0.378 |
| A_51_P102081 | NM_007847 | 13222 | Defcr-rs2 | 0.061 | 0.0426 | 0.0644 | 0.0791 | 0.158 | 0.0864 |
| A_51_P285725 | NM_009309 | 20997 | T | 0.141 | 0.102 | 0.113 | 0.134 | 0.13 | 0.141 |
| A_52_P827911 | AK081941; | | | 2.387 | 2.657 | 2.47 | 2.075 | 2.44 | 2.116 |
| A_51_P231341 | AK087764; | 108160 | D0HXS9928E | 2.885 | 3.795 | 2.39 | 3.305 | 3.578 | 2.451 |
| A_51_P334093 | NM_026228 | 67547 | Slc39a8 | 0.208 | 0.0771 | 0.105 | 0.19 | 0.198 | 0.231 |
| A_52_P196568 | NM_144513 | 17263 | Gtl2 | 1.892 | 2.538 | 1.516 | 1.938 | 1.959 | 1.652 |
| A_52_P1108139 | AK053079; | | | 1.801 | 2.214 | 1.538 | 1.924 | 2.168 | 1.724 |
| A_52_P636559 | AK053008; | 268469 | 9530033F24Rik | 2.63 | 2.537 | 2.752 | 2.132 | 2.334 | 1.998 |
| A_52_P425734 | NM_145146 | 280662 | Afm | 3.913 | 3.546 | 3.74 | 3.764 | 4.135 | 3.335 |
| A_52_P1011737 | AK039096; | 106258 | AI790442 | 1.846 | 1.977 | 1.978 | 2.087 | 2.67 | 2.065 |
| A_52_P537140 | AK047235; | | | 1.74 | 2.015 | 2.044 | 1.613 | 1.708 | 1.261 |
| A_51_P191990 | AK087548; | 26563 | Ror1 | 1.628 | 1.623 | 1.626 | 1.509 | 1.588 | 1.479 |
| A_51_P478930 | AK080016; | | | 2.436 | 2.701 | 2.337 | 2.779 | 2.919 | 2.466 |
| A_51_P252565 | NM_009660 | 11687 | Alox15 | 0.236 | 0.24 | 0.156 | 0.217 | 0.231 | 0.212 |
| A_52_P81252 | NM_172599 | 218978 | D14Ertd436e | 2.478 | 2.667 | 2.607 | 1.82 | 1.981 | 1.764 |
| A_51_P487004 | NM_027732 | 71241 | Dmrtc2 | 10.3 | 11.69 | 10.98 | 5.024 | 6.192 | 5.685 |
| A_51_P104162 | AK053379; | 328232 | | 2.537 | 3.096 | 3.391 | 3.126 | 2.998 | 3.127 |
| A_52_P555603 | AK032770; | 11787 | Apbb2 | 1.409 | 1.222 | 1.29 | 1.326 | 1.427 | 1.18 |
| A_52_P819356 | AK041684; | | | 3.003 | 2.848 | 2.7 | 3.026 | 2.915 | 3.006 |
| A_52_P1196751 | AK086488; | | | 2.217 | 2.193 | 2.198 | 2.38 | 2.677 | 2.156 |
| A_52_P277415 | AK077313; | 76252 | 0610006O14Rik | 1.282 | 1.291 | 1.224 | 0.99 | 1.044 | 0.981 |
| A_52_P812362 | BC016205; | 408064 | BC064078 | 0.25 | 0.167 | 0.217 | 0.302 | 0.27 | 0.27 |
| A_51_P352572 | NM_019701 | 56365 | Clcnkb | 5.193 | 4.267 | 4.344 | 4.514 | 5.226 | 4.635 |
| A_52_P100042 | AK045143; | 320938 | Tnpo3 | 1.761 | 1.695 | 1.74 | 1.697 | 1.665 | 1.526 |
| A_51_P103397 | AK078521; | 54561 | Nap1l3 | 2.701 | 2.645 | 2.885 | 3.303 | 3.612 | 3.031 |
| A_52_P296244 | BC048734; | | | 0.369 | 0.373 | 0.292 | 0.457 | 0.42 | 0.426 |
| A_51_P305019 | AK020601; | 78423 | 9530049O05Rik | 1.297 | 1.416 | 1.484 | 1.43 | 1.551 | 1.387 |
| A_52_P3304 | NM_080446 | 117599 | Helb | 2.823 | 2.774 | 3.121 | 2.206 | 2.73 | 2.462 |
| A_51_P147651 | AK041797; XM_146705 | 245902 | | 2.797 | 2.965 | 2.694 | 2.18 | 2.325 | 1.652 |
| A_51_P203955 | NM_010260 | 14469 | Gbp2 | 0.252 | 0.267 | 0.292 | 0.582 | 0.578 | 0.664 |
| A_51_P176474 | AK010466; | 76484 | 2410012C07Rik | 2.797 | 2.691 | 2.733 | 3.87 | 4.072 | 3.726 |
| A_52_P165497 | NM_011385 | 20481 | Ski | 2.312 | 2.37 | 2.474 | 1.296 | 1.485 | 1.406 |
| A_51_P358714 | AK031433; | | | 2.043 | 2.448 | 1.68 | 2.647 | 2.627 | 1.899 |

TABLE 3B
200 Gene Subset (Non Germline Competent)

| | | | | Clone 1 | | | Clone 2 | | |
|---|---|---|---|---|---|---|---|---|---|
| Agilent Probe ID | Genbank | Entrez ID | Gene Symbol | rep. 1 | rep. 2 | rep. 3 | rep. 1 | rep. 2 | rep. 3 |
| A_51_P237040 | NM_008711 | 18121 | Nog | 1.152 | 1.407 | 1.101 | 0.859 | 0.96 | 0.924 |
| A_51_P335460 | NM_009132 | 20259 | Scin | 1.658 | 1.913 | 1.519 | 1.101 | 1.225 | 1.107 |
| A_52_P391505 | NM_144513 | 17263 | Gtl2 | 0.0793 | 0.114 | 0.0867 | 0.158 | 0.15 | 0.148 |
| A_51_P497100 | NM_010706 | 16855 | Lgals4 | 0.692 | 0.771 | 0.849 | 1.002 | 0.908 | 0.828 |
| A_51_P378371 | | | | 0.986 | 1.006 | 1.208 | 1.217 | 1.156 | 1.234 |
| A_51_P298266 | NM_025681 | 66643 | Lix1 | 1.123 | 1.145 | 1.109 | 1.143 | 1.15 | 1.141 |
| A_52_P269158 | NM_001003948 | 98496 | AL024069 | 1.775 | 1.419 | 1.506 | 1.494 | 1.391 | 1.254 |
| A_51_P311038 | BC009666; XM_134720 | 57267 | Apba3 | 1.847 | 1.642 | 1.538 | 2.55 | 2.405 | 2.808 |
| A_51_P457187 | NM_028075 | 72049 | Tnfrsf13c | 1.083 | 1.58 | 1.373 | 0.916 | 1.099 | 1.091 |
| A_52_P68702 | NM_145148 | 232288 | Frmd4b | 0.928 | 1.071 | 0.877 | 1.242 | 1.234 | 1.139 |
| A_52_P293443 | AK087208; | 13819 | | 0.179 | 0.222 | 0.219 | 0.543 | 0.565 | 0.552 |
| A_51_P157986 | NM_029930 | 77574 | 3321401G04Rik | 0.909 | 1.196 | 1.088 | 0.563 | 0.621 | 0.598 |
| A_52_P1013441 | | | | 1.255 | 1.032 | 2.041 | 1.173 | 1.203 | 1.341 |
| A_51_P418375 | NM_023844 | 67374 | Jam2 | 1.044 | 0.943 | 0.99 | 0.826 | 0.786 | 0.84 |
| A_52_P557940 | AK004302; | 66704 | 4921506I22Rik | 0.544 | 0.595 | 0.569 | 1.371 | 1.379 | 1.241 |
| A_51_P480311 | NM_010168 | 14061 | F2 | 0.348 | 0.476 | 0.622 | 0.996 | 1.004 | 1.186 |
| A_52_P200458 | NM_010242 | 14345 | Fut4 | 1.003 | 0.997 | 1.017 | 1.108 | 1.203 | 1.09 |
| A_51_P463846 | NM_145545 | 229900 | 9830147J24Rik | 2.28 | 2.199 | 2.49 | 2.068 | 1.958 | 1.72 |
| A_51_P162474 | AF285577; | 107815 | Scml2 | 2.885 | 2.549 | 2.371 | 3.021 | 3.116 | 3.1 |
| A_51_P165244 | NM_018734 | 55932 | Gbp4 | 2.388 | 1.319 | 2.543 | 2.404 | 2.051 | 2.534 |
| A_52_P59318 | NM_133832 | 98711 | Rdh10 | 1.689 | 1.611 | 1.584 | 0.876 | 1.002 | 0.955 |
| A_52_P438036 | NM_175271 | 78134 | Gpr23 | 1.082 | 0.96 | 0.968 | 1.634 | 1.593 | 1.618 |
| A_52_P474242 | BE457768; | | | 1.31 | 1.785 | 1.55 | 1.137 | 1.132 | 0.997 |
| A_51_P499854 | NM_010284 | 14600 | Ghr | 1.55 | 1.19 | 1.486 | 2.502 | 2.537 | 2.196 |
| A_51_P455647 | NM_009801 | 12349 | Car2 | 1.592 | 1.463 | 1.337 | 2.139 | 2.347 | 2.17 |
| A_52_P103929 | NM_009306 | 20979 | Syt1 | 0.898 | 0.938 | 0.967 | 1.626 | 1.464 | 1.422 |
| A_51_P149469 | NM_009608 | 11464 | Actc1 | 1.012 | 1.006 | 0.935 | 0.971 | 0.997 | 1.12 |
| A_52_P557293 | NM_015775 | 50528 | Tmprss2 | 1.142 | 1.091 | 1.111 | 1.208 | 1.127 | 1.225 |
| A_52_P50496 | AY048587; XM_619453 | 114640 | Tifp39 | 1.196 | 1.551 | 1.504 | 1.114 | 1.171 | 1.214 |
| A_51_P180413 | NM_176930 | 319504 | C130076O07Rik | 1.603 | 1.54 | 1.395 | 1.297 | 1.25 | 1.155 |
| A_51_P229613 | NM_008792 | 18549 | Pcsk2 | 1.463 | 1.53 | 1.37 | 1.333 | 1.422 | 1.554 |
| A_51_P121635 | NM_030700 | 80884 | Maged2 | 1.225 | 1.426 | 1.335 | 0.918 | 0.99 | 0.88 |
| A_51_P481777 | NM_018756 | 54382 | Tcstv1 | 0.208 | 0.696 | 0.654 | 0.592 | 0.503 | 0.72 |
| A_51_P412817 | NM_177617 | 216851 | D330014H01Rik | 0.725 | 0.834 | 1.04 | 1.083 | 0.986 | 0.997 |
| A_51_P387528 | AK019664; | 78801 | Ak7 | 0.65 | 0.537 | 0.813 | 0.767 | 0.778 | 1.11 |
| A_52_P78168 | AK031518; | | | 0.277 | 0.385 | 0.299 | 0.704 | 0.652 | 0.544 |
| A_52_P274960 | NM_001005508 | 226652 | 6030405P05Rik | 0.671 | 0.634 | 0.712 | 0.763 | 0.781 | 0.808 |
| A_52_P1188270 | AK042362; | | | 0.341 | 0.594 | 0.564 | 1.758 | 1.462 | 1.658 |
| A_51_P291227 | NM_001005508 | 226652 | 6030405P05Rik | 0.705 | 0.681 | 0.731 | 0.814 | 0.735 | 0.798 |
| A_52_P377299 | AK048360; | 271377 | Zbtb11 | 0.577 | 0.582 | 0.645 | 1.152 | 1.092 | 1.023 |
| A_51_P297131 | NM_009502 | 22330 | Vcl | 1.015 | 1.436 | 0.955 | 0.556 | 0.672 | 0.587 |
| A_52_P804224 | | | | 0.299 | 0.314 | 0.291 | 1.263 | 1.2 | 1.068 |
| A_51_P442894 | AK048310; | 621603 | C130048D07Rik | 1.023 | 0.841 | 1.218 | 0.788 | 0.867 | 0.977 |
| A_52_P309718 | AK173099; | 218850 | D14Abb1e | 1.006 | 0.813 | 1.127 | 1.186 | 1.29 | 1.289 |
| A_52_P359010 | AK129478; | 72193 | Sfrs2ip | 0.54 | 0.706 | 0.574 | 1.484 | 1.398 | 1.403 |
| A_51_P410813 | AK079732; | | | 0.717 | 0.877 | 0.904 | 1.593 | 1.473 | 1.607 |
| A_52_P382565 | XM_483943 | 432493 | LOC432493 | 0.228 | 0.273 | 0.254 | 0.607 | 0.622 | 0.462 |
| A_51_P333253 | NM_178440 | 246177 | Myo1g | 0.535 | 0.491 | 0.538 | 0.474 | 0.481 | 0.37 |
| A_52_P113190 | NM_053214 | 17916 | Myo1f | 0.386 | 0.513 | 0.397 | 0.243 | 0.232 | 0.264 |
| A_51_P473953 | XM_358600; XM_355454 | 381498 | 4930592C13Rik | 2.138 | 1.593 | 1.947 | 2.323 | 2.226 | 2.26 |
| A_52_P519870 | AB010340; | | | 0.758 | 0.675 | 1.128 | 0.989 | 0.863 | 1.393 |
| A_51_P266403 | AK009163; | 76730 | 2310005C01Rik | 0.984 | 0.901 | 1.009 | 1.273 | 1.313 | 1.391 |
| A_52_P3698 | NM_001015099 | 217558 | | 0.658 | 0.653 | 0.676 | 1.487 | 1.564 | 1.537 |
| A_52_P581100 | NM_027285 | 70005 | 1700029I01Rik | 1.164 | 1.1 | 1.141 | 1.525 | 1.383 | 1.305 |
| A_51_P276305 | NM_009292 | 20899 | Stra8 | 0.873 | 0.934 | 0.982 | 0.431 | 0.426 | 0.471 |
| A_51_P455997 | Y13832; | 17263 | Gtl2 | 0.123 | 0.184 | 0.114 | 0.213 | 0.197 | 0.258 |
| A_52_P656336 | NM_023733 | 74114 | Crot | 1.233 | 1.547 | 1.442 | 0.983 | 1.067 | 0.998 |
| A_51_P512172 | AK010953; XM_125673 | 52463 | Cxxc6 | 0.8 | 0.986 | 0.748 | 1.033 | 1.014 | 1.046 |
| A_51_P282227 | NM_020577 | 57344 | As3mt | 0.79 | 0.761 | 0.777 | 1.02 | 0.91 | 0.98 |
| A_52_P386544 | NM_026594 | 68172 | 4930517K11Rik | 0.74 | 0.606 | 0.729 | 1.188 | 1.159 | 1.351 |
| A_51_P125679 | AK010725; XM_134948 | 73673 | 2410076I21Rik | 0.588 | 0.959 | 0.916 | 0.84 | 0.843 | 0.849 |
| A_51_P164504 | NM_007469 | 11812 | Apoc1 | 1.307 | 1.245 | 1.314 | 1.412 | 1.375 | 1.457 |
| A_52_P240706 | NM_007543 | 26367 | Ceacam2 | 0.87 | 0.778 | 0.892 | 0.689 | 0.756 | 0.859 |
| A_52_P420369 | NM_029444 | 75801 | 4930447C04Rik | 1.087 | 1.084 | 1.129 | 1.176 | 0.989 | 1.041 |
| A_51_P242399 | NM_031170 | 16691 | Krt2-8 | 1.013 | 1.199 | 0.939 | 0.962 | 0.977 | 0.946 |
| A_51_P158037 | NM_013505 | 13506 | Dsc2 | 1.238 | 1.562 | 1.304 | 0.743 | 0.819 | 0.875 |
| A_52_P258116 | NM_009521 | 22415 | Wnt3 | 1.304 | 1.249 | 1.083 | 1.811 | 1.943 | 2.006 |
| A_52_P286166 | NM_001002927 | 18619 | Penk1 | 1.649 | 2.509 | 2.439 | 1.542 | 1.453 | 1.374 |
| A_52_P399095 | NM_177793 | 327747 | 9030224M15Rik | 1.11 | 0.976 | 1.056 | 1.583 | 1.692 | 1.596 |
| A_52_P470663 | | | | 2.339 | 2.72 | 2.405 | 1.069 | 1.105 | 1.018 |
| A_52_P376169 | NM_177139 | 320343 | E130115E03Rik | 1.184 | 1.025 | 1.076 | 1.949 | 1.81 | 1.828 |
| A_51_P512085 | NM_130449 | 140792 | Colec12 | 1.574 | 1.25 | 1.471 | 1.463 | 1.651 | 1.693 |
| A_52_P676403 | NM_019494 | 56066 | Cxcl11 | 3.86 | 3.177 | 3.663 | 2.834 | 2.723 | 2.826 |
| A_51_P354307 | NM_172469 | 209195 | Clic6 | 1.31 | 1.578 | 1.57 | 0.719 | 0.748 | 0.659 |
| A_52_P569375 | NM_010203 | 14176 | Fgf5 | 4.338 | 3.206 | 3.958 | 5.889 | 5.927 | 6.75 |
| A_51_P162481 | NM_133194 | 107815 | Scml2 | 2.434 | 2.616 | 2.232 | 2.312 | 2.394 | 2.596 |
| A_51_P202911 | NM_144882 | 67198 | 2810022L02Rik | 1.005 | 0.995 | 0.908 | 0.913 | 0.931 | 0.902 |
| A_51_P418029 | NM_175541 | 245631 | Mum1l1 | 2.026 | 1.803 | 2.159 | 1.492 | 1.654 | 1.714 |
| A_52_P93837 | NM_008604 | 17380 | Mme | 1.516 | 1.805 | 1.761 | 1.442 | 1.369 | 1.329 |
| A_51_P275435 | NM_172411 | 71874 | 2310007B03Rik | 3.355 | 3.105 | 3.53 | 5.636 | 6.155 | 6.288 |
| A_51_P199888 | NM_026082 | 67299 | Dock7 | 1.182 | 1.246 | 1.295 | 0.669 | 0.678 | 1 |
| A_52_P211956 | NM_025658 | 66607 | Ms4a4d | 1.71 | 1.799 | 1.851 | 2.105 | 2.274 | 1.961 |
| A_51_P485985 | NM_134163 | 171170 | Mbnl3 | 1.781 | 1.802 | 1.648 | 0.929 | 1.067 | 1.035 |
| A_52_P630964 | NM_027533 | 70747 | Tspan2 | 1.217 | 1.314 | 1.26 | 0.815 | 0.863 | 0.897 |
| A_51_P294505 | NM_025658 | 66607 | Ms4a4d | 1.546 | 1.551 | 1.883 | 2.127 | 2.066 | 2.183 |
| A_52_P82488 | NM_021476 | 58861 | Cysltr1 | 1.919 | 1.474 | 1.811 | 2.106 | 2.164 | 2.621 |
| A_52_P634829 | NM_023835 | 76681 | Trim12 | 1.52 | 1.676 | 1.775 | 1.472 | 1.69 | 1.577 |
| A_52_P460882 | XM_130319; XM_130308 | 73668; 14725 | Ttc21b | 1.498 | 1.698 | 2.077 | 1.06 | 1.276 | 1.219 |
| A_52_P197826 | NM_010397 | 15039 | H2-T22 | 1.502 | 1.687 | 1.444 | 1.028 | 1.049 | 1.039 |
| A_51_P240253 | NM_019662 | 56437 | Rrad | 1.392 | 1.648 | 1.29 | 0.851 | 0.9 | 0.913 |
| A_51_P101975 | NM_010205 | 14179 | Fgf8 | 2.683 | 2.96 | 2.139 | 3.25 | 3.463 | 3.854 |
| A_52_P21595 | NM_028238 | 72433 | Rab38 | 1.043 | 0.791 | 0.992 | 0.807 | 0.819 | 0.929 |
| A_52_P402786 | NM_008935 | 19126 | Prom1 | 1.498 | 1.71 | 1.749 | 1.028 | 1.023 | 0.92 |
| A_52_P126158 | NM_008326 | 15944 | Ifi1 | 1.157 | 1.034 | 1.006 | 1.254 | 1.3 | 1.232 |
| A_52_P676510 | NM_011579 | 21822 | Tgtp | 1.166 | 1.03 | 1.064 | 1.15 | 1.119 | 1.082 |
| A_52_P175685 | NM_008390 | 16362 | Irf1 | 1.072 | 1.33 | 1.162 | 0.971 | 1.106 | 1.017 |
| A_52_P384718 | BC052056; XM_283264 | 320873 | | 1.488 | 1.451 | 1.435 | 1.051 | 1.032 | 1.026 |
| A_52_P176333 | NM_001005787 | 12695 | Cipp | 1.168 | 1.146 | 1.249 | 0.974 | 1.014 | 1.036 |
| A_52_P358951 | AK081999; XM_134736 | 271457; 22688 | Rab5a | 1.346 | 1.445 | 1.326 | 0.843 | 0.883 | 0.787 |
| A_51_P168646 | AK039580; | | | 1.655 | 1.373 | 1.436 | 2.357 | 2.478 | 2.267 |
| A_52_P620345 | NM_133979 | 102566 | AI604832 | 1.26 | 1.488 | 1.375 | 0.778 | 0.752 | 0.729 |
| A_52_P253179 | AK077477; | 16009 | Igfbp3 | 2.157 | 1.536 | 2.035 | 1.993 | 1.965 | 2.545 |
| A_51_P397003 | XM_133990 | 68904 | 1110065L07Rik | 1.235 | 1.343 | 1.238 | 0.778 | 0.763 | 0.855 |
| A_52_P74441 | NM_011570 | 21753 | Tes | 1.498 | 1.167 | 1.425 | 0.853 | 0.882 | 0.885 |
| A_52_P255250 | NM_009543 | 22644 | Rnf103 | 1.205 | 1.217 | 1.524 | 0.663 | 0.701 | 0.687 |
| A_52_P575505 | NM_008994 | 19302 | Pxmp3 | 1.123 | 1.07 | 1.092 | 0.797 | 0.815 | 0.802 |
| A_52_P469789 | NM_007588 | 12311 | Calcr | 2.087 | 2.206 | 2.278 | 1.646 | 1.638 | 1.533 |
| A_51_P345422 | NM_010747 | 17096 | Lyn | 0.888 | 0.803 | 0.881 | 1.006 | 0.995 | 0.89 |
| A_51_P356003 | NM_001007580 | 333564 | Gm784 | 1.223 | 1.221 | 1.186 | 1.543 | 1.618 | 1.409 |
| A_52_P680941 | NM_010284 | 14600 | Ghr | 1.72 | 1.563 | 1.682 | 2.058 | 1.978 | 2.065 |
| A_52_P380263 | NM_013723 | 27205 | Podxl | 1.179 | 1.159 | 1.66 | 0.899 | 0.909 | 0.942 |
| A_52_P286528 | BC055771; | 102954 | Nudt11 | 1.419 | 1.344 | 1.431 | 1.048 | 1.063 | 1.053 |
| A_51_P105068 | NM_027990 | 71897 | 2310010M24Rik | 1.902 | 1.982 | 1.72 | 2.012 | 2.165 | 2.002 |
| A_52_P132608 | NM_172685 | 229731 | Slc25a24 | 0.809 | 0.906 | 0.807 | 0.708 | 0.722 | 0.693 |
| A_51_P467505 | NM_013689 | 21682 | Tec | 1.077 | 1.062 | 1.153 | 0.842 | 0.861 | 0.881 |
| A_52_P153685 | AK129323; | 70422 | 2810417D08Rik | 1.763 | 1.676 | 1.845 | 2.204 | 2.099 | 2.085 |
| A_51_P174943 | NM_008485 | 16782 | Lamc2 | 1.282 | 1.255 | 1.249 | 1.068 | 1.04 | 1.004 |
| A_52_P301821 | NM_053078 | 27528 | D0H4S114 | 1.076 | 1.275 | 1.138 | 0.88 | 0.828 | 0.741 |
| A_52_P532910 | NM_024427 | 22003 | Tpm1 | 1.081 | 1.106 | 1.238 | 0.891 | 0.87 | 0.851 |
| A_51_P521084 | NM_025626 | 66540 | 3110001A13Rik | 1.423 | 1.425 | 1.474 | 0.832 | 0.865 | 0.776 |
| A_52_P680436 | NM_025793 | 66840 | Wdr45l | 1.338 | 1.213 | 1.346 | 0.755 | 0.702 | 0.805 |
| A_51_P164219 | NM_011909 | 24110 | Usp18 | 1.876 | 1.698 | 1.64 | 1.445 | 1.463 | 1.349 |
| A_52_P421234 | NM_133832 | 98711 | Rdh10 | 1.706 | 1.519 | 1.676 | 0.845 | 0.856 | 0.776 |
| A_52_P586157 | NM_022309 | 12400 | Cbfb | 1.417 | 1.551 | 1.338 | 0.773 | 0.863 | 0.903 |
| A_51_P262171 | NM_008326 | 15944 | Ifi1 | 1.271 | 1.011 | 1.135 | 1.457 | 1.571 | 1.485 |
| A_52_P452689 | NM_007498 | 11910 | Atf3 | 0.672 | 0.61 | 0.659 | 0.89 | 0.955 | 0.943 |
| A_52_P295933 | AK045060; | 14051 | B130023L16Rik | 1.637 | 1.973 | 2.08 | 1.007 | 0.993 | 1.019 |
| A_51_P222773 | NM_010446 | 15376 | Foxa2 | 2.333 | 1.693 | 1.64 | 6.867 | 6.289 | 6.137 |
| A_52_P361534 | NM_009521 | 22415 | Wnt3 | 1.193 | 0.891 | 0.969 | 2.247 | 2.459 | 2.231 |
| A_51_P507880 | NM_028980 | 74521 | 8430415E04Rik | 2.104 | 2.169 | 2.081 | 2.44 | 2.499 | 2.391 |
| A_52_P574668 | NM_011851 | 23959 | Nt5e | 1.611 | 1.127 | 1.284 | 1.764 | 1.707 | 1.826 |
| A_52_P957500 | XM_622354 | | | 0.371 | 0.532 | 0.397 | 1.528 | 1.372 | 1.305 |
| A_52_P345626 | AY149175; | 386626 | Mzf6d | 0.699 | 0.636 | 0.76 | 1.173 | 1.052 | 1.267 |
| A_52_P84015 | AK037762; | 11863 | | 0.695 | 0.924 | 0.842 | 2.965 | 3.344 | 3.023 |
| A_52_P432464 | AK053863; | 192196 | Luc7l2 | 0.7 | 0.863 | 1.032 | 1.457 | 1.269 | 1.582 |
| A_52_P376502 | BC025011; | 227541 | Camk1d | 0.664 | 0.71 | 0.737 | 0.973 | 0.893 | 1.008 |
| A_52_P354785 | AK037929; | 14852 | Gspt1 | 0.487 | 0.598 | 0.585 | 1.587 | 1.447 | 1.483 |
| A_51_P398766 | NM_010259 | 14468 | Gbp1 | 3.558 | 2.476 | 3.155 | 4.099 | 3.953 | 3.971 |
| A_52_P486234 | AK033497; | 22680 | | 0.658 | 0.943 | 0.756 | 2.133 | 2.185 | 1.908 |
| A_52_P628212 | AK044339; | 26380 | | 1.058 | 1.337 | 0.967 | 4.002 | 3.276 | 3.435 |
| A_52_P445799 | AK046777; | 52490 | D18Ertd289e | 2.088 | 2.22 | 2.899 | 2.952 | 2.963 | 3.531 |
| A_51_P268167 | BC037232; XM_621365 | 229363; 218850 | Gmps | 0.465 | 0.448 | 0.523 | 1.267 | 1.053 | 1.205 |
| A_51_P153079 | NM_133923 | 101100 | 4833441J24Rik | 0.42 | 0.232 | 0.278 | 1.153 | 1.158 | 1.047 |
| A_52_P584874 | NM_152895 | 75605 | Jarid1b | 0.737 | 0.83 | 0.752 | 1.242 | 1.251 | 1.242 |
| A_52_P307801 | NM_007847 | 13222 | Defcr-rs2 | 2.149 | 1.602 | 1.28 | 2.942 | 3.1 | 2.825 |
| A_52_P163148 | AB010370; | 544945 | | 0.704 | 0.687 | 0.76 | 1.098 | 1.144 | 1.325 |
| A_51_P171728 | NM_011926 | 26365 | Ceacam1 | 0.474 | 0.703 | 0.686 | 0.748 | 0.952 | 0.943 |
| A_51_P141660 | AK053665; XM_358382 | 67776; 235380 | Loh11cr2a | 0.43 | 0.62 | 0.908 | 1.844 | 1.422 | 1.976 |
| A_52_P532323 | NM_178410 | 218518 | Marveld2 | 1.824 | 2.102 | 1.904 | 0.871 | 0.884 | 1.027 |
| A_51_P337125 | NM_010566 | 16331 | Inpp5d | 0.938 | 0.873 | 1.036 | 1.261 | 1.272 | 1.297 |
| A_52_P336259 | AK087367; XM_485576 | 319467 | A930002C04Rik | 0.552 | 0.507 | 0.623 | 1.053 | 0.913 | 0.8 |
| A_51_P318577 | AK010729; | 73671 | 2410078J06Rik | 0.605 | 0.863 | 0.641 | 0.897 | 1.028 | 1.15 |
| A_52_P324934 | AK045391; | 16403 | | 0.758 | 0.573 | 0.774 | 1.808 | 1.513 | 1.534 |
| A_52_P826666 | AK007019; | 51799 | Rap2ip | 0.894 | 1.131 | 1.14 | 2.913 | 2.479 | 3.106 |
| A_51_P200223 | XM_619497 | | | 0.65 | 0.646 | 0.76 | 1.392 | 1.367 | 1.332 |
| A_51_P228472 | NM_008343 | 16009 | Igfbp3 | 1.472 | 2.26 | 1.637 | 1.02 | 1.11 | 1.239 |
| A_52_P206573 | NM_027427 | 70439 | Taf15 | 0.525 | 0.7 | 0.806 | 1.685 | 1.521 | 1.76 |
| A_52_P354363 | NM_026574 | 68142 | | 0.545 | 0.546 | 0.579 | 1.246 | 1.113 | 1.072 |
| A_52_P651469 | NM_001012726 | 503692 | | 1.087 | 2.041 | 1.585 | 2.114 | 1.954 | 2.198 |
| A_51_P463789 | BC051947; | 58212 | | 0.924 | 0.962 | 0.995 | 1.518 | 1.581 | 1.46 |
| A_52_P18887 | NM_011468 | 20755 | Sprr2a | 7.008 | 6.235 | 5.038 | 6.207 | 6.689 | 6.331 |
| A_51_P223132 | NM_031183; XM_486227 | 83395; 70549 | Sp6 | 0.649 | 0.542 | 0.552 | 1.095 | 0.998 | 1.125 |
| A_52_P223083 | BC038323; | 72096 | 2010208K18Rik | 0.89 | 0.64 | 1.018 | 2.024 | 1.641 | 2.309 |
| A_52_P523399 | NM_029444 | 75801 | 4930447C04Rik | 1.167 | 0.873 | 1.364 | 1.373 | 1.087 | 1.771 |
| A_52_P282000 | AK020248; | 13800 | Enah | 0.368 | 0.482 | 0.376 | 1.791 | 1.63 | 1.536 |
| A_52_P152983 | AK011739; | 26886 | Cenph | 0.423 | 0.805 | 0.28 | 1.393 | 1.582 | 1.833 |
| A_52_P26416 | AF106279; | 16782 | | 1.436 | 1.802 | 1.779 | 0.723 | 0.806 | 0.697 |
| A_51_P102081 | NM_007847 | 13222 | Defcr-rs2 | 2.408 | 2.238 | 1.498 | 3.38 | 3.255 | 2.763 |
| A_51_P285725 | NM_009309 | 20997 | T | 2.672 | 1.6 | 1.512 | 3.129 | 3.214 | 3.267 |
| A_52_P827911 | AK081941; | | | 0.209 | 0.476 | 0.487 | 1.73 | 1.566 | 1.778 |
| A_51_P231341 | AK087764; | 108160 | D0HXS9928E | 0.131 | 0.284 | 0.334 | 1.584 | 1 | 1.654 |
| A_51_P334093 | NM_026228 | 67547 | Slc39a8 | 2.173 | 2.638 | 2.466 | 1.07 | 1.006 | 1.061 |
| A_52_P196568 | NM_144513 | 17263 | Gtl2 | 0.0697 | 0.0369 | 0.0417 | 0.112 | 0.0801 | 0.115 |
| A_52_P1108139 | AK053079; | | | 0.169 | 0.348 | 0.199 | 1.068 | 1.372 | 1.161 |
| A_52_P636559 | AK053008; | 268469 | 9530033F24Rik | 0.506 | 0.53 | 0.567 | 1.485 | 1.451 | 1.363 |
| A_52_P425734 | NM_145146 | 280662 | Afm | 1.466 | 1.63 | 2.105 | 1.902 | 1.789 | 2.093 |
| A_52_P1011737 | AK039096; | 106258 | AI790442 | 0.224 | 0.44 | 0.438 | 1.367 | 1.308 | 1.503 |
| A_52_P537140 | AK047235; | | | 0.566 | 0.565 | 0.531 | 1.029 | 1.127 | 1.156 |
| A_51_P191990 | AK087548; | 26563 | Ror1 | 0.378 | 0.368 | 0.389 | 1.197 | 1.149 | 1.176 |
| A_51_P478930 | AK080016; | | | 0.64 | 0.623 | 0.816 | 1.097 | 1.039 | 1.17 |
| A_51_P252565 | NM_009660 | 11687 | Alox15 | 1.618 | 1.425 | 1.194 | 1.366 | 1.436 | 1.457 |
| A_52_P81252 | NM_172599 | 218978 | D14Ertd436e | 0.265 | 0.408 | 0.488 | 1.153 | 1.046 | 0.927 |
| A_51_P487004 | NM_027732 | 71241 | Dmrtc2 | 1.918 | 2.991 | 1.94 | 1.77 | 1.537 | 1.901 |
| A_51_P104162 | AK053379; | 328232 | | 0.351 | 0.468 | 0.481 | 1.289 | 1.159 | 1.082 |
| A_52_P555603 | AK032770; | 11787 | Apbb2 | 0.273 | 0.355 | 0.304 | 1.021 | 1.021 | 1.054 |
| A_52_P819356 | AK041684; | | | 0.651 | 0.704 | 0.763 | 1.654 | 1.657 | 1.821 |
| A_52_P1196751 | AK086488; | | | 0.314 | 0.63 | 0.521 | 1.285 | 1.098 | 1.263 |
| A_52_P277415 | AK077313; | 76252 | 0610006O14Rik | 0.0165 | 0.0277 | 0.0223 | 0.0497 | 0.0428 | 0.0552 |
| A_52_P812362 | BC016205; | 408064 | BC064078 | 1.424 | 1.508 | 1.444 | 1.323 | 1.468 | 1.262 |
| A_51_P352572 | NM_019701 | 56365 | Clcnkb | 1.441 | 1.565 | 1.422 | 1.707 | 1.362 | 1.314 |
| A_52_P100042 | AK045143; | 320938 | Tnpo3 | 0.301 | 0.507 | 0.406 | 1.444 | 1.417 | 1.439 |
| A_51_P103397 | AK078521; | 54561 | Nap1l3 | 0.942 | 0.996 | 1.004 | 1.047 | 0.978 | 1.076 |
| A_52_P296244 | BC048734; | | | 2.333 | 2.799 | 1.851 | 2.371 | 2.371 | 2.372 |
| A_51_P305019 | AK020601; | 78423 | 9530049O05Rik | 0.201 | 0.4 | 0.429 | 1.187 | 1.059 | 1.249 |
| A_52_P3304 | NM_080446 | 117599 | Helb | 1.004 | 1.239 | 1.059 | 1.614 | 1.432 | 1.418 |
| A_51_P147651 | AK041797; XM_146705 | 245902 | | 0.511 | 0.729 | 0.963 | 1.47 | 1.214 | 1.502 |
| A_51_P203955 | NM_010260 | 14469 | Gbp2 | 2.194 | 2.061 | 2.214 | 2.45 | 2.681 | 2.345 |
| A_51_P176474 | AK010466; | 76484 | 2410012C07Rik | 0.842 | 0.903 | 1.004 | 0.996 | 1.036 | 1.127 |
| A_52_P165497 | NM_011385 | 20481 | Ski | 0.541 | 0.665 | 0.649 | 1.004 | 0.817 | 0.972 |
| A_51_P358714 | AK031433; | | | 0.23 | 0.294 | 0.418 | 1.005 | 0.775 | 1.214 |

TABLE 4A
100 Gene Subset (Germline Competent)

| | | | | Clone 1 | | | Clone 2 | | |
|---|---|---|---|---|---|---|---|---|---|
| Agilent Probe ID | Genbank | Entrez ID | Gene Symbol | rep. 1 | rep. 2 | rep. 3 | rep. 1 | rep. 2 | rep. 3 |
| A_51_P237040 | NM_008711 | 18121 | Nog | 0.266 | 0.299 | 0.281 | 0.396 | 0.368 | 0.407 |
| A_51_P335460 | NM_009132 | 20259 | Scin | 0.718 | 0.631 | 0.719 | 0.678 | 0.674 | 0.666 |
| A_52_P391505 | NM_144513 | 17263 | Gtl2 | 1.36 | 1.291 | 1.327 | 1.053 | 1.04 | 0.852 |
| A_51_P497100 | NM_010706 | 16855 | Lgals4 | 2.115 | 2.049 | 2.368 | 1.716 | 1.861 | 1.527 |
| A_51_P378371 | | | | 0.454 | 0.542 | 0.39 | 0.468 | 0.484 | 0.468 |
| A_51_P298266 | NM_025681 | 66643 | Lix1 | 0.507 | 0.414 | 0.416 | 0.554 | 0.584 | 0.54 |
| A_52_P269158 | NM_001003948 | 98496 | AL024069 | 0.316 | 0.33 | 0.33 | 0.388 | 0.393 | 0.443 |
| A_51_P311038 | BC009666; XM_134720 | 57267 | Apba3 | 0.334 | 0.341 | 0.339 | 0.446 | 0.472 | 0.482 |
| A_51_P457187 | NM_028075 | 72049 | Tnfrsf13c | 2.82 | 3.275 | 3.026 | 2.654 | 3.432 | 2.546 |
| A_52_P68702 | NM_145148 | 232288 | Frmd4b | 0.545 | 0.476 | 0.538 | 0.501 | 0.43 | 0.424 |
| A_52_P293443 | AK087208; | 13819 | | 1.278 | 1.075 | 1.13 | 1.496 | 1.604 | 1.349 |
| A_51_P157986 | NM_029930 | 77574 | 3321401G04Rik | 0.426 | 0.339 | 0.345 | 0.368 | 0.318 | 0.37 |
| A_52_P1013441 | | | | 3.248 | 3.164 | 3.191 | 4.558 | 5.61 | 4.683 |
| A_51_P418375 | NM_023844 | 67374 | Jam2 | 1.691 | 1.467 | 1.625 | 2.142 | 2.132 | 2.288 |
| A_52_P557940 | AK004302; | 66704 | 4921506I22Rik | 2.114 | 1.863 | 2.221 | 2.17 | 2.013 | 2.051 |
| A_51_P480311 | NM_010168 | 14061 | F2 | 1.991 | 1.809 | 1.969 | 2.465 | 2.4 | 1.874 |
| A_52_P200458 | NM_010242 | 14345 | Fut4 | 0.429 | 0.343 | 0.355 | 0.453 | 0.391 | 0.512 |
| A_51_P463846 | NM_145545 | 229900 | 9830147J24Rik | 0.694 | 0.519 | 0.59 | 0.86 | 0.816 | 0.817 |
| A_51_P162474 | AF285577; | 107815 | Scml2 | 0.539 | 0.413 | 0.405 | 0.643 | 0.645 | 0.71 |
| A_51_P165244 | NM_018734 | 55932 | Gbp4 | 0.585 | 0.512 | 0.474 | 1.008 | 0.981 | 1.006 |
| A_52_P59318 | NM_133832 | 98711 | Rdh10 | 0.306 | 0.273 | 0.275 | 0.373 | 0.395 | 0.445 |
| A_52_P438036 | NM_175271 | 78134 | Gpr23 | 0.376 | 0.334 | 0.36 | 0.455 | 0.441 | 0.489 |
| A_52_P474242 | BE457768; | | | 0.499 | 0.53 | 0.46 | 0.69 | 0.658 | 0.726 |
| A_51_P499854 | NM_010284 | 14600 | Ghr | 0.407 | 0.375 | 0.412 | 0.413 | 0.429 | 0.489 |
| A_51_P455647 | NM_009801 | 12349 | Car2 | 0.335 | 0.288 | 0.301 | 0.568 | 0.516 | 0.638 |
| A_52_P103929 | NM_009306 | 20979 | Syt1 | 0.382 | 0.356 | 0.39 | 0.419 | 0.498 | 0.423 |
| A_51_P149469 | NM_009608 | 11464 | Actc1 | 0.324 | 0.387 | 0.384 | 0.496 | 0.551 | 0.471 |
| A_52_P557293 | NM_015775 | 50528 | Tmprss2 | 0.277 | 0.205 | 0.179 | 0.45 | 0.457 | 0.355 |
| A_52_P50496 | AY048587; XM_619453 | 114640 | Tifp39 | 0.454 | 0.501 | 0.422 | 0.661 | 0.638 | 0.694 |
| A_51_P180413 | NM_176930 | 319504 | C130076O07Rik | 0.359 | 0.393 | 0.415 | 0.471 | 0.514 | 0.494 |
| A_51_P229613 | NM_008792 | 18549 | Pcsk2 | 0.512 | 0.483 | 0.515 | 0.58 | 0.58 | 0.504 |
| A_51_P121635 | NM_030700 | 80884 | Maged2 | 0.378 | 0.371 | 0.409 | 0.492 | 0.475 | 0.481 |
| A_51_P481777 | NM_018756 | 54382 | Tcstv1 | 1.532 | 1.822 | 1.792 | 2.619 | 3.155 | 2.956 |
| A_51_P412817 | NM_177617 | 216851 | D330014H01Rik | 3.151 | 3.039 | 3.326 | 2.136 | 2.194 | 1.865 |
| A_51_P387528 | AK019664; | 78801 | Ak7 | 2.693 | 2.814 | 2.57 | 3.786 | 3.834 | 3.71 |
| A_52_P78168 | AK031518; | | | 2.108 | 1.677 | 1.617 | 1.783 | 1.791 | 1.711 |
| A_52_P274960 | NM_001005508 | 226652 | 6030405P05Rik | 1.83 | 1.82 | 1.948 | 2.07 | 2.181 | 2.076 |
| A_52_P1188270 | AK042362; | | | 3.076 | 1.828 | 1.917 | 2.932 | 3.046 | 2.87 |
| A_51_P291227 | NM_001005508 | 226652 | 6030405P05Rik | 1.876 | 1.826 | 1.86 | 2.054 | 2.185 | 2.004 |
| A_52_P377299 | AK048360; | 271377 | Zbtb11 | 1.927 | 1.892 | 1.923 | 1.678 | 1.79 | 1.546 |
| A_51_P297131 | NM_009502 | 22330 | Vcl | 0.402 | 0.372 | 0.378 | 0.411 | 0.377 | 0.401 |
| A_52_P804224 | | | | 1.522 | 1.688 | 1.828 | 2.31 | 2.487 | 2.241 |
| A_51_P442894 | AK048310; | 621603 | C130048D07Rik | 2.632 | 2.364 | 2.281 | 3.657 | 3.714 | 3.102 |
| A_52_P309718 | AK173099; | 218850 | D14Abb1e | 2.324 | 2.579 | 2.18 | 2.614 | 2.72 | 2.711 |
| A_52_P359010 | AK129478; | 72193 | Sfrs2ip | 2.152 | 2.149 | 2.122 | 2.349 | 2.38 | 2.318 |
| A_51_P410813 | AK079732; | | | 2.732 | 2.716 | 2.559 | 2.898 | 2.605 | 2.3 |
| A_52_P382565 | XM_483943 | 432493 | LOC432493 | 0.976 | 1.222 | 1.221 | 1.492 | 1.439 | 1.198 |
| A_51_P333253 | NM_178440 | 246177 | Myo1g | 1.419 | 1.413 | 1.621 | 1.544 | 1.504 | 1.394 |
| A_52_P113190 | NM_053214 | 17916 | Myo1f | 1.428 | 1.513 | 1.396 | 1.988 | 1.959 | 2.083 |
| A_51_P473953 | XM_358600; XM_355454 | 381498 | 4930592C13Rik | 0.42 | 0.306 | 0.298 | 0.365 | 0.365 | 0.38 |
| A_52_P519870 | AB010340; | | | 2.391 | 3.34 | 2.621 | 3.76 | 3.72 | 1.955 |
| A_51_P266403 | AK009163; | 76730 | 2310005C01Rik | 3.079 | 3.053 | 3.092 | 2.733 | 2.967 | 2.734 |
| A_52_P3698 | NM_001015099 | 217558 | | 1.885 | 1.84 | 1.762 | 2.392 | 2.296 | 2.486 |
| A_52_P581100 | NM_027285 | 70005 | 1700029I01Rik | 2.295 | 3.266 | 2.708 | 3.168 | 3.146 | 2.879 |
| A_51_P276305 | NM_009292 | 20899 | Stra8 | 3.137 | 3.069 | 3.174 | 2.824 | 2.972 | 2.777 |
| A_51_P455997 | Y13832; | 17263 | Gtl2 | 1.505 | 1.455 | 1.44 | 0.998 | 1.082 | 0.998 |
| A_52_P656336 | NM_023733 | 74114 | Crot | 0.407 | 0.351 | 0.381 | 0.755 | 0.647 | 0.8 |
| A_51_P512172 | AK010953; XM_125673 | 52463 | Cxxc6 | 2.864 | 2.384 | 2.216 | 2.184 | 2.11 | 2.068 |
| A_51_P282227 | NM_020577 | 57344 | As3mt | 2.651 | 2.607 | 2.876 | 3.224 | 3.327 | 3.147 |
| A_52_P386544 | NM_026594 | 68172 | 4930517K11Rik | 2.843 | 2.618 | 2.802 | 2.673 | 2.913 | 2.894 |
| A_51_P125679 | AK010725; XM_134948 | 73673 | 2410076I21Rik | 2.867 | 2.332 | 2.464 | 3.518 | 3.661 | 3.727 |
| A_51_P164504 | NM_007469 | 11812 | Apoc1 | 3.245 | 2.908 | 3.052 | 2.863 | 3.059 | 2.591 |
| A_52_P240706 | NM_007543 | 26367 | Ceacam2 | 2.285 | 1.866 | 1.892 | 2.654 | 2.556 | 2.628 |
| A_52_P420369 | NM_029444 | 75801 | 4930447C04Rik | 3.256 | 2.433 | 2.971 | 2.042 | 2.189 | 2.206 |
| A_51_P242399 | NM_031170 | 16691 | Krt2-8 | 0.193 | 0.233 | 0.208 | 0.403 | 0.4 | 0.39 |
| A_51_P158037 | NM_013505 | 13506 | Dsc2 | 0.271 | 0.249 | 0.258 | 0.437 | 0.415 | 0.453 |
| A_52_P258116 | NM_009521 | 22415 | Wnt3 | 0.156 | 0.164 | 0.16 | 0.312 | 0.309 | 0.296 |
| A_52_P286166 | NM_001002927 | 18619 | Penk1 | 0.497 | 0.324 | 0.339 | 0.671 | 0.658 | 0.647 |
| A_52_P399095 | NM_177793 | 327747 | 9030224M15Rik | 0.366 | 0.325 | 0.163 | 0.433 | 0.401 | 0.466 |
| A_52_P470663 | | | | 0.377 | 0.334 | 0.403 | 0.467 | 0.439 | 0.566 |
| A_52_P376169 | NM_177139 | 320343 | E130115E03Rik | 0.314 | 0.285 | 0.295 | 0.539 | 0.508 | 0.528 |
| A_51_P512085 | NM_130449 | 140792 | Colec12 | 0.335 | 0.364 | 0.296 | 0.382 | 0.368 | 0.385 |
| A_52_P676403 | NM_019494 | 56066 | Cxcl11 | 0.525 | 0.596 | 0.597 | 0.761 | 0.809 | 0.704 |
| A_51_P354307 | NM_172469 | 209195 | Clic6 | 0.43 | 0.319 | 0.329 | 0.398 | 0.398 | 0.387 |
| A_52_P569375 | NM_010203 | 14176 | Fgf5 | 0.776 | 0.678 | 0.554 | 0.45 | 0.467 | 0.574 |
| A_51_P162481 | NM_133194 | 107815 | Scml2 | 0.432 | 0.357 | 0.385 | 0.594 | 0.55 | 0.618 |
| A_51_P202911 | NM_144882 | 67198 | 2810022L02Rik | 0.297 | 0.283 | 0.328 | 0.346 | 0.333 | 0.365 |
| A_51_P418029 | NM_175541 | 245631 | Mum1l1 | 0.319 | 0.265 | 0.278 | 0.419 | 0.397 | 0.534 |
| A_52_P93837 | NM_008604 | 17380 | Mme | 0.559 | 0.439 | 0.513 | 0.653 | 0.573 | 0.747 |
| A_51_P275435 | NM_172411 | 71874 | 2310007B03Rik | 0.368 | 0.492 | 0.206 | 0.875 | 0.83 | 0.857 |
| A_51_P199888 | NM_026082 | 67299 | Dock7 | 0.407 | 0.403 | 0.245 | 0.558 | 0.428 | 0.546 |
| A_52_P211956 | NM_025658 | 66607 | Ms4a4d | 0.61 | 0.455 | 0.457 | 0.534 | 0.519 | 0.685 |
| A_51_P485985 | NM_134163 | 171170 | Mbnl3 | 0.223 | 0.181 | 0.161 | 0.625 | 0.543 | 0.697 |
| A_52_P630964 | NM_027533 | 70747 | Tspan2 | 0.361 | 0.283 | 0.324 | 0.499 | 0.473 | 0.525 |
| A_51_P294505 | NM_025658 | 66607 | Ms4a4d | 0.63 | 0.414 | 0.365 | 0.532 | 0.521 | 0.629 |
| A_52_P82488 | NM_021476 | 58861 | Cysltr1 | 0.319 | 0.379 | 0.306 | 0.5 | 0.438 | 0.534 |
| A_52_P634829 | NM_023835 | 76681 | Trim12 | 0.59 | 0.561 | 0.672 | 1.012 | 0.996 | 0.866 |
| A_52_P460882 | XM_130319; XM_130308 | 73668; 14725 | Ttc21b | 0.412 | 0.637 | 0.533 | 0.396 | 0.633 | 0.502 |
| A_52_P197826 | NM_010397 | 15039 | H2-T22 | 0.541 | 0.505 | 0.41 | 0.653 | 0.634 | 0.622 |
| A_51_P240253 | NM_019662 | 56437 | Rrad | 0.316 | 0.322 | 0.314 | 0.392 | 0.337 | 0.342 |
| A_51_P101975 | NM_010205 | 14179 | Fgf8 | 0.412 | 0.39 | 0.413 | 0.415 | 0.454 | 0.385 |
| A_52_P21595 | NM_028238 | 72433 | Rab38 | 0.234 | 0.188 | 0.168 | 0.439 | 0.467 | 0.472 |
| A_52_P402786 | NM_008935 | 19126 | Prom1 | 0.37 | 0.396 | 0.335 | 0.551 | 0.506 | 0.594 |
| A_52_P126158 | NM_008326 | 15944 | Ifi1 | 0.237 | 0.208 | 0.22 | 0.415 | 0.412 | 0.425 |
| A_52_P676510 | NM_011579 | 21822 | Tgtp | 0.225 | 0.162 | 0.155 | 0.382 | 0.378 | 0.362 |
| A_52_P175685 | NM_008390 | 16362 | Irf1 | 0.401 | 0.37 | 0.3 | 0.663 | 0.637 | 0.749 |
| A_52_P384718 | BC052056; XM_283264 | 320873 | | 0.508 | 0.441 | 0.458 | 0.541 | 0.512 | 0.578 |
| A_52_P176333 | NM_001005787 | 12695 | Cipp | 0.372 | 0.357 | 0.365 | 0.452 | 0.392 | 0.42 |
| A_52_P358951 | AK081999; XM_134736 | 271457; 22688 | Rab5a | 0.417 | 0.378 | 0.448 | 0.464 | 0.449 | 0.46 |
| A_51_P168646 | AK039580; | | | 0.442 | 0.434 | 0.509 | 0.537 | 0.548 | 0.598 |

TABLE 4B
100 Gene Subset (Non Germline Competent)

| | | | | Clone 1 | | | Clone 2 | | |
|---|---|---|---|---|---|---|---|---|---|
| Agilent Probe ID | Genbank | Entrez ID | Gene Symbol | rep. 1 | rep. 2 | rep. 3 | rep. 1 | rep. 2 | rep. 3 |
| A_51_P237040 | NM_008711 | 18121 | Nog | 1.152 | 1.407 | 1.101 | 0.859 | 0.96 | 0.924 |
| A_51_P335460 | NM_009132 | 20259 | Scin | 1.658 | 1.913 | 1.519 | 1.101 | 1.225 | 1.107 |
| A_52_P391505 | NM_144513 | 17263 | Gtl2 | 0.0793 | 0.114 | 0.0867 | 0.158 | 0.15 | 0.148 |
| A_51_P497100 | NM_010706 | 16855 | Lgals4 | 0.692 | 0.771 | 0.849 | 1.002 | 0.908 | 0.828 |
| A_51_P378371 | | | | 0.986 | 1.006 | 1.208 | 1.217 | 1.156 | 1.234 |
| A_51_P298266 | NM_025681 | 66643 | Lix1 | 1.123 | 1.145 | 1.109 | 1.143 | 1.15 | 1.141 |
| A_52_P269158 | NM_001003948 | 98496 | AL024069 | 1.775 | 1.419 | 1.506 | 1.494 | 1.391 | 1.254 |
| A_51_P311038 | BC009666; XM_134720 | 57267 | Apba3 | 1.847 | 1.642 | 1.538 | 2.55 | 2.405 | 2.808 |
| A_51_P457187 | NM_028075 | 72049 | Tnfrsf13c | 1.083 | 1.58 | 1.373 | 0.916 | 1.099 | 1.091 |
| A_52_P68702 | NM_145148 | 232288 | Frmd4b | 0.928 | 1.071 | 0.877 | 1.242 | 1.234 | 1.139 |
| A_52_P293443 | AK087208; | 13819 | | 0.179 | 0.222 | 0.219 | 0.543 | 0.565 | 0.552 |
| A_51_P157986 | NM_029930 | 77574 | 3321401G04Rik | 0.909 | 1.196 | 1.088 | 0.563 | 0.621 | 0.598 |
| A_52_P1013441 | | | | 1.255 | 1.032 | 2.041 | 1.173 | 1.203 | 1.341 |
| A_51_P418375 | NM_023844 | 67374 | Jam2 | 1.044 | 0.943 | 0.99 | 0.826 | 0.786 | 0.84 |
| A_52_P557940 | AK004302; | 66704 | 4921506I22Rik | 0.544 | 0.595 | 0.569 | 1.371 | 1.379 | 1.241 |
| A_51_P480311 | NM_010168 | 14061 | F2 | 0.348 | 0.476 | 0.622 | 0.996 | 1.004 | 1.186 |
| A_52_P200458 | NM_010242 | 14345 | Fut4 | 1.003 | 0.997 | 1.017 | 1.108 | 1.203 | 1.09 |
| A_51_P463846 | NM_145545 | 229900 | 9830147J24Rik | 2.28 | 2.199 | 2.49 | 2.068 | 1.958 | 1.72 |
| A_51_P162474 | AF285577; | 107815 | Scml2 | 2.885 | 2.549 | 2.371 | 3.021 | 3.116 | 3.1 |
| A_51_P165244 | NM_018734 | 55932 | Gbp4 | 2.388 | 1.319 | 2.543 | 2.404 | 2.051 | 2.534 |
| A_52_P59318 | NM_133832 | 98711 | Rdh10 | 1.689 | 1.611 | 1.584 | 0.876 | 1.002 | 0.955 |
| A_52_P438036 | NM_175271 | 78134 | Gpr23 | 1.082 | 0.96 | 0.968 | 1.634 | 1.593 | 1.618 |
| A_52_P474242 | BE457768; | | | 1.31 | 1.785 | 1.55 | 1.137 | 1.132 | 0.997 |
| A_51_P499854 | NM_010284 | 14600 | Ghr | 1.55 | 1.19 | 1.486 | 2.502 | 2.537 | 2.196 |
| A_51_P455647 | NM_009801 | 12349 | Car2 | 1.592 | 1.463 | 1.337 | 2.139 | 2.347 | 2.17 |
| A_52_P103929 | NM_009306 | 20979 | Syt1 | 0.898 | 0.938 | 0.967 | 1.626 | 1.464 | 1.422 |
| A_51_P149469 | NM_009608 | 11464 | Actc1 | 1.012 | 1.006 | 0.935 | 0.971 | 0.997 | 1.12 |
| A_52_P557293 | NM_015775 | 50528 | Tmprss2 | 1.142 | 1.091 | 1.111 | 1.208 | 1.127 | 1.225 |
| A_52_P50496 | AY048587; XM_619453 | 114640 | Tifp39 | 1.196 | 1.551 | 1.504 | 1.114 | 1.171 | 1.214 |
| A_51_P180413 | NM_176930 | 319504 | C130076O07Rik | 1.603 | 1.54 | 1.395 | 1.297 | 1.25 | 1.155 |
| A_51_P229613 | NM_008792 | 18549 | Pcsk2 | 1.463 | 1.53 | 1.37 | 1.333 | 1.422 | 1.554 |
| A_51_P121635 | NM_030700 | 80884 | Maged2 | 1.225 | 1.426 | 1.335 | 0.918 | 0.99 | 0.88 |
| A_51_P481777 | NM_018756 | 54382 | Tcstv1 | 0.208 | 0.696 | 0.654 | 0.592 | 0.503 | 0.72 |
| A_51_P412817 | NM_177617 | 216851 | D330014H01Rik | 0.725 | 0.834 | 1.04 | 1.083 | 0.986 | 0.997 |
| A_51_P387528 | AK019664; | 78801 | Ak7 | 0.65 | 0.537 | 0.813 | 0.767 | 0.778 | 1.11 |
| A_52_P78168 | AK031518; | | | 0.277 | 0.385 | 0.299 | 0.704 | 0.652 | 0.544 |
| A_52_P274960 | NM_001005508 | 226652 | 6030405P05Rik | 0.671 | 0.634 | 0.712 | 0.763 | 0.781 | 0.808 |
| A_52_P1188270 | AK042362; | | | 0.341 | 0.594 | 0.564 | 1.758 | 1.462 | 1.658 |
| A_51_P291227 | NM_001005508 | 226652 | 6030405P05Rik | 0.705 | 0.681 | 0.731 | 0.814 | 0.735 | 0.798 |
| A_52_P377299 | AK048360; | 271377 | Zbtb11 | 0.577 | 0.582 | 0.645 | 1.152 | 1.092 | 1.023 |
| A_51_P297131 | NM_009502 | 22330 | Vcl | 1.015 | 1.436 | 0.955 | 0.556 | 0.672 | 0.587 |
| A_52_P804224 | | | | 0.299 | 0.314 | 0.291 | 1.263 | 1.2 | 1.068 |
| A_51_P442894 | AK048310; | 621603 | C130048D07Rik | 1.023 | 0.841 | 1.218 | 0.788 | 0.867 | 0.977 |
| A_52_P309718 | AK173099; | 218850 | D14Abb1e | 1.006 | 0.813 | 1.127 | 1.186 | 1.29 | 1.289 |
| A_52_P359010 | AK129478; | 72193 | Sfrs2ip | 0.54 | 0.706 | 0.574 | 1.484 | 1.398 | 1.403 |
| A_51_P410813 | AK079732; | | | 0.717 | 0.877 | 0.904 | 1.593 | 1.473 | 1.607 |
| A_52_P382565 | XM_483943 | 432493 | LOC432493 | 0.228 | 0.273 | 0.254 | 0.607 | 0.622 | 0.462 |
| A_51_P333253 | NM_178440 | 246177 | Myo1g | 0.535 | 0.491 | 0.538 | 0.474 | 0.481 | 0.37 |
| A_52_P113190 | NM_053214 | 17916 | Myo1f | 0.386 | 0.513 | 0.397 | 0.243 | 0.232 | 0.264 |
| A_51_P473953 | XM_358600; XM_355454 | 381498 | 4930592C13Rik | 2.138 | 1.593 | 1.947 | 2.323 | 2.226 | 2.26 |
| A_52_P519870 | AB010340; | | | 0.758 | 0.675 | 1.128 | 0.989 | 0.863 | 1.393 |
| A_51_P266403 | AK009163; | 76730 | 2310005C01Rik | 0.984 | 0.901 | 1.009 | 1.273 | 1.313 | 1.391 |
| A_52_P3698 | NM_001015099 | 217558 | | 0.658 | 0.653 | 0.676 | 1.487 | 1.564 | 1.537 |
| A_52_P581100 | NM_027285 | 70005 | 1700029I01Rik | 1.164 | 1.1 | 1.141 | 1.525 | 1.383 | 1.305 |
| A_51_P276305 | NM_009292 | 20899 | Stra8 | 0.873 | 0.934 | 0.982 | 0.431 | 0.426 | 0.471 |
| A_51_P455997 | Y13832; | 17263 | Gtl2 | 0.123 | 0.184 | 0.114 | 0.213 | 0.197 | 0.258 |
| A_52_P656336 | NM_023733 | 74114 | Crot | 1.233 | 1.547 | 1.442 | 0.983 | 1.067 | 0.998 |
| A_51_P512172 | AK010953; XM_125673 | 52463 | Cxxc6 | 0.8 | 0.986 | 0.748 | 1.033 | 1.014 | 1.046 |
| A_51_P282227 | NM_020577 | 57344 | As3mt | 0.79 | 0.761 | 0.777 | 1.02 | 0.91 | 0.98 |
| A_52_P386544 | NM_026594 | 68172 | 4930517K11Rik | 0.74 | 0.606 | 0.729 | 1.188 | 1.159 | 1.351 |
| A_51_P125679 | AK010725; XM_134948 | 73673 | 2410076I21Rik | 0.588 | 0.959 | 0.916 | 0.84 | 0.843 | 0.849 |
| A_51_P164504 | NM_007469 | 11812 | Apoc1 | 1.307 | 1.245 | 1.314 | 1.412 | 1.375 | 1.457 |
| A_52_P240706 | NM_007543 | 26367 | Ceacam2 | 0.87 | 0.778 | 0.892 | 0.689 | 0.756 | 0.859 |
| A_52_P420369 | NM_029444 | 75801 | 4930447C04Rik | 1.087 | 1.084 | 1.129 | 1.176 | 0.989 | 1.041 |
| A_51_P242399 | NM_031170 | 16691 | Krt2-8 | 1.013 | 1.199 | 0.939 | 0.962 | 0.977 | 0.946 |
| A_51_P158037 | NM_013505 | 13506 | Dsc2 | 1.238 | 1.562 | 1.304 | 0.743 | 0.819 | 0.875 |
| A_52_P258116 | NM_009521 | 22415 | Wnt3 | 1.304 | 1.249 | 1.083 | 1.811 | 1.943 | 2.006 |
| A_52_P286166 | NM_001002927 | 18619 | Penk1 | 1.649 | 2.509 | 2.439 | 1.542 | 1.453 | 1.374 |
| A_52_P399095 | NM_177793 | 327747 | 9030224M15Rik | 1.11 | 0.976 | 1.056 | 1.583 | 1.692 | 1.596 |
| A_52_P470663 | | | | 2.339 | 2.72 | 2.405 | 1.069 | 1.105 | 1.018 |
| A_52_P376169 | NM_177139 | 320343 | E130115E03Rik | 1.184 | 1.025 | 1.076 | 1.949 | 1.81 | 1.828 |
| A_51_P512085 | NM_130449 | 140792 | Colec12 | 1.574 | 1.25 | 1.471 | 1.463 | 1.651 | 1.693 |
| A_52_P676403 | NM_019494 | 56066 | Cxcl11 | 3.86 | 3.177 | 3.663 | 2.834 | 2.723 | 2.826 |
| A_51_P354307 | NM_172469 | 209195 | Clic6 | 1.31 | 1.578 | 1.57 | 0.719 | 0.748 | 0.659 |
| A_52_P569375 | NM_010203 | 14176 | Fgf5 | 4.338 | 3.206 | 3.958 | 5.889 | 5.927 | 6.75 |
| A_51_P162481 | NM_133194 | 107815 | Scml2 | 2.434 | 2.616 | 2.232 | 2.312 | 2.394 | 2.596 |
| A_51_P202911 | NM_144882 | 67198 | 2810022L02Rik | 1.005 | 0.995 | 0.908 | 0.913 | 0.931 | 0.902 |
| A_51_P418029 | NM_175541 | 245631 | Mum1l1 | 2.026 | 1.803 | 2.159 | 1.492 | 1.654 | 1.714 |
| A_52_P93837 | NM_008604 | 17380 | Mme | 1.516 | 1.805 | 1.761 | 1.442 | 1.369 | 1.329 |
| A_51_P275435 | NM_172411 | 71874 | 2310007B03Rik | 3.355 | 3.105 | 3.53 | 5.636 | 6.155 | 6.288 |
| A_51_P199888 | NM_026082 | 67299 | Dock7 | 1.182 | 1.246 | 1.295 | 0.669 | 0.678 | 1 |
| A_52_P211956 | NM_025658 | 66607 | Ms4a4d | 1.71 | 1.799 | 1.851 | 2.105 | 2.274 | 1.961 |
| A_51_P485985 | NM_134163 | 171170 | Mbnl3 | 1.781 | 1.802 | 1.648 | 0.929 | 1.067 | 1.035 |
| A_52_P630964 | NM_027533 | 70747 | Tspan2 | 1.217 | 1.314 | 1.26 | 0.815 | 0.863 | 0.897 |
| A_51_P294505 | NM_025658 | 66607 | Ms4a4d | 1.546 | 1.551 | 1.883 | 2.127 | 2.066 | 2.183 |
| A_52_P82488 | NM_021476 | 58861 | Cysltr1 | 1.919 | 1.474 | 1.811 | 2.106 | 2.164 | 2.621 |
| A_52_P634829 | NM_023835 | 76681 | Trim12 | 1.52 | 1.676 | 1.775 | 1.472 | 1.69 | 1.577 |
| A_52_P460882 | XM_130319; XM_130308 | 73668; 14725 | Ttc21b | 1.498 | 1.698 | 2.077 | 1.06 | 1.276 | 1.219 |
| A_52_P197826 | NM_010397 | 15039 | H2-T22 | 1.502 | 1.687 | 1.444 | 1.028 | 1.049 | 1.039 |
| A_51_P240253 | NM_019662 | 56437 | Rrad | 1.392 | 1.648 | 1.29 | 0.851 | 0.9 | 0.913 |
| A_51_P101975 | NM_010205 | 14179 | Fgf8 | 2.683 | 2.96 | 2.139 | 3.25 | 3.463 | 3.854 |
| A_52_P21595 | NM_028238 | 72433 | Rab38 | 1.043 | 0.791 | 0.992 | 0.807 | 0.819 | 0.929 |
| A_52_P402786 | NM_008935 | 19126 | Prom1 | 1.498 | 1.71 | 1.749 | 1.028 | 1.023 | 0.92 |
| A_52_P126158 | NM_008326 | 15944 | Ifi1 | 1.157 | 1.034 | 1.006 | 1.254 | 1.3 | 1.232 |
| A_52_P676510 | NM_011579 | 21822 | Tgtp | 1.166 | 1.03 | 1.064 | 1.15 | 1.119 | 1.082 |
| A_52_P175685 | NM_008390 | 16362 | Irf1 | 1.072 | 1.33 | 1.162 | 0.971 | 1.106 | 1.017 |
| A_52_P384718 | BC052056; XM_283264 | 320873 | | 1.488 | 1.451 | 1.435 | 1.051 | 1.032 | 1.026 |
| A_52_P176333 | NM_001005787 | 12695 | Cipp | 1.168 | 1.146 | 1.249 | 0.974 | 1.014 | 1.036 |
| A_52_P358951 | AK081999; XM_134736 | 271457; 22688 | Rab5a | 1.346 | 1.445 | 1.326 | 0.843 | 0.883 | 0.787 |
| A_51_P168646 | AK039580; | | | 1.655 | 1.373 | 1.436 | 2.357 | 2.478 | 2.267 |

TABLE 5A
50 Gene Subset (Germline Competent)

| | | | | Clone 1 | | | Clone 2 | | |
|---|---|---|---|---|---|---|---|---|---|
| Agilent Probe ID | Genbank | Entrez ID | Gene Symbol | rep. 1 | rep. 2 | rep. 3 | rep. 1 | rep. 2 | rep. 3 |
| A_51_P237040 | NM_008711 | 18121 | Nog | 0.266 | 0.299 | 0.281 | 0.396 | 0.368 | 0.407 |
| A_51_P335460 | NM_009132 | 20259 | Scin | 0.718 | 0.631 | 0.719 | 0.678 | 0.674 | 0.666 |
| A_52_P391505 | NM_144513 | 17263 | Gtl2 | 1.36 | 1.291 | 1.327 | 1.053 | 1.04 | 0.852 |
| A_51_P497100 | NM_010706 | 16855 | Lgals4 | 2.115 | 2.049 | 2.368 | 1.716 | 1.861 | 1.527 |
| A_51_P378371 | | | | 0.454 | 0.542 | 0.39 | 0.468 | 0.484 | 0.468 |
| A_51_P298266 | NM_025681 | 66643 | Lix1 | 0.507 | 0.414 | 0.416 | 0.554 | 0.584 | 0.54 |
| A_52_P269158 | NM_001003948 | 98496 | AL024069 | 0.316 | 0.33 | 0.33 | 0.388 | 0.393 | 0.443 |
| A_51_P311038 | BC009666; XM_134720 | 57267 | Apba3 | 0.334 | 0.341 | 0.339 | 0.446 | 0.472 | 0.482 |
| A_51_P457187 | NM_028075 | 72049 | Tnfrsf13c | 2.82 | 3.275 | 3.026 | 2.654 | 3.432 | 2.546 |
| A_52_P68702 | NM_145148 | 232288 | Frmd4b | 0.545 | 0.476 | 0.538 | 0.501 | 0.43 | 0.424 |
| A_52_P293443 | AK087208; | 13819 | | 1.278 | 1.075 | 1.13 | 1.496 | 1.604 | 1.349 |
| A_51_P157986 | NM_029930 | 77574 | 3321401G04Rik | 0.426 | 0.339 | 0.345 | 0.368 | 0.318 | 0.37 |
| A_52_P1013441 | | | | 3.248 | 3.164 | 3.191 | 4.558 | 5.61 | 4.683 |
| A_51_P418375 | NM_023844 | 67374 | Jam2 | 1.691 | 1.467 | 1.625 | 2.142 | 2.132 | 2.288 |
| A_52_P557940 | AK004302; | 66704 | 4921506I22Rik | 2.114 | 1.863 | 2.221 | 2.17 | 2.013 | 2.051 |
| A_51_P480311 | NM_010168 | 14061 | F2 | 1.991 | 1.809 | 1.969 | 2.465 | 2.4 | 1.874 |
| A_52_P200458 | NM_010242 | 14345 | Fut4 | 0.429 | 0.343 | 0.355 | 0.453 | 0.391 | 0.512 |
| A_51_P463846 | NM_145545 | 229900 | 9830147J24Rik | 0.694 | 0.519 | 0.59 | 0.86 | 0.816 | 0.817 |
| A_51_P162474 | AF285577; | 107815 | Scml2 | 0.539 | 0.413 | 0.405 | 0.643 | 0.645 | 0.71 |
| A_51_P165244 | NM_018734 | 55932 | Gbp4 | 0.585 | 0.512 | 0.474 | 1.008 | 0.981 | 1.006 |
| A_52_P59318 | NM_133832 | 98711 | Rdh10 | 0.306 | 0.273 | 0.275 | 0.373 | 0.395 | 0.445 |
| A_52_P438036 | NM_175271 | 78134 | Gpr23 | 0.376 | 0.334 | 0.36 | 0.455 | 0.441 | 0.489 |
| A_52_P474242 | BE457768; | | | 0.499 | 0.53 | 0.46 | 0.69 | 0.658 | 0.726 |
| A_51_P499854 | NM_010284 | 14600 | Ghr | 0.407 | 0.375 | 0.412 | 0.413 | 0.429 | 0.489 |
| A_51_P455647 | NM_009801 | 12349 | Car2 | 0.335 | 0.288 | 0.301 | 0.568 | 0.516 | 0.638 |
| A_52_P103929 | NM_009306 | 20979 | Syt1 | 0.382 | 0.356 | 0.39 | 0.419 | 0.498 | 0.423 |
| A_51_P149469 | NM_009608 | 11464 | Actc1 | 0.324 | 0.387 | 0.384 | 0.496 | 0.551 | 0.471 |
| A_52_P557293 | NM_015775 | 50528 | Tmprss2 | 0.277 | 0.205 | 0.179 | 0.45 | 0.457 | 0.355 |
| A_52_P50496 | AY048587; XM_619453 | 114640 | Tifp39 | 0.454 | 0.501 | 0.422 | 0.661 | 0.638 | 0.694 |
| A_51_P180413 | NM_176930 | 319504 | C130076O07Rik | 0.359 | 0.393 | 0.415 | 0.471 | 0.514 | 0.494 |
| A_51_P229613 | NM_008792 | 18549 | Pcsk2 | 0.512 | 0.483 | 0.515 | 0.58 | 0.58 | 0.504 |
| A_51_P121635 | NM_030700 | 80884 | Maged2 | 0.378 | 0.371 | 0.409 | 0.492 | 0.475 | 0.481 |
| A_51_P481777 | NM_018756 | 54382 | Tcstv1 | 1.532 | 1.822 | 1.792 | 2.619 | 3.155 | 2.956 |
| A_51_P412817 | NM_177617 | 216851 | D330014H01Rik | 3.151 | 3.039 | 3.326 | 2.136 | 2.194 | 1.865 |
| A_51_P387528 | AK019664; | 78801 | Ak7 | 2.693 | 2.814 | 2.57 | 3.786 | 3.834 | 3.71 |
| A_52_P78168 | AK031518; | | | 2.108 | 1.677 | 1.617 | 1.783 | 1.791 | 1.711 |
| A_52_P274960 | NM_001005508 | 226652 | 6030405P05Rik | 1.83 | 1.82 | 1.948 | 2.07 | 2.181 | 2.076 |
| A_52_P1188270 | AK042362; | | | 3.076 | 1.828 | 1.917 | 2.932 | 3.046 | 2.87 |
| A_51_P291227 | NM_001005508 | 226652 | 6030405P05Rik | 1.876 | 1.826 | 1.86 | 2.054 | 2.185 | 2.004 |
| A_52_P377299 | AK048360; | 271377 | Zbtb11 | 1.927 | 1.892 | 1.923 | 1.678 | 1.79 | 1.546 |
| A_51_P297131 | NM_009502 | 22330 | Vcl | 0.402 | 0.372 | 0.378 | 0.411 | 0.377 | 0.401 |
| A_52_P804224 | | | | 1.522 | 1.688 | 1.828 | 2.31 | 2.487 | 2.241 |
| A_51_P442894 | AK048310; | 621603 | C130048D07Rik | 2.632 | 2.364 | 2.281 | 3.657 | 3.714 | 3.102 |
| A_52_P309718 | AK173099; | 218850 | D14Abb1e | 2.324 | 2.579 | 2.18 | 2.614 | 2.72 | 2.711 |
| A_52_P359010 | AK129478; | 72193 | Sfrs2ip | 2.152 | 2.149 | 2.122 | 2.349 | 2.38 | 2.318 |
| A_51_P410813 | AK079732; | | | 2.732 | 2.716 | 2.559 | 2.898 | 2.605 | 2.3 |
| A_52_P382565 | XM_483943 | 432493 | LOC432493 | 0.976 | 1.222 | 1.221 | 1.492 | 1.439 | 1.198 |
| A_51_P333253 | NM_178440 | 246177 | Myo1g | 1.419 | 1.413 | 1.621 | 1.544 | 1.504 | 1.394 |
| A_52_P113190 | NM_053214 | 17916 | Myo1f | 1.428 | 1.513 | 1.396 | 1.988 | 1.959 | 2.083 |
| A_51_P473953 | XM_358600; XM_355454 | 381498 | 4930592C13Rik | 0.42 | 0.306 | 0.298 | 0.365 | 0.365 | 0.38 |

TABLE 5B
50 Gene Subset (Non Germline Competent)

| | |
|---|---|
| Clone 1 | Clone 2 |

| Agilent Probe ID | Genbank | Entrez ID | Gene Symbol | rep. 1 | rep. 2 | rep. 3 | rep. 1 | rep. 2 | rep. 3 |
|---|---|---|---|---|---|---|---|---|---|
| A_51_P237040 | NM_008711 | 18121 | Nog | 1.152 | 1.407 | 1.101 | 0.859 | 0.96 | 0.924 |
| A_51_P335460 | NM_009132 | 20259 | Scin | 1.658 | 1.913 | 1.519 | 1.101 | 1.225 | 1.107 |
| A_52_P391505 | NM_144513 | 17263 | Gtl2 | 0.0793 | 0.114 | 0.0867 | 0.158 | 0.15 | 0.148 |
| A_51_P497100 | NM_010706 | 16855 | Lgals4 | 0.692 | 0.771 | 0.849 | 1.002 | 0.908 | 0.828 |
| A_51_P378371 | | | | 0.986 | 1.006 | 1.208 | 1.217 | 1.156 | 1.234 |
| A_51_P298266 | NM_025681 | 66643 | Lix1 | 1.123 | 1.145 | 1.109 | 1.143 | 1.15 | 1.141 |
| A_52_P269158 | NM_001003948 | 98496 | AL024069 | 1.775 | 1.419 | 1.506 | 1.494 | 1.391 | 1.254 |
| A_51_P311038 | BC009666; XM_134720 | 57267 | Apba3 | 1.847 | 1.642 | 1.538 | 2.55 | 2.405 | 2.808 |
| A_51_P457187 | NM_028075 | 72049 | Tnfrsf13c | 1.083 | 1.58 | 1.373 | 0.916 | 1.099 | 1.091 |
| A_52_P68702 | NM_145148 | 232288 | Frmd4b | 0.928 | 1.071 | 0.877 | 1.242 | 1.234 | 1.139 |
| A_52_P293443 | AK087208; | 13819 | | 0.179 | 0.222 | 0.219 | 0.543 | 0.565 | 0.552 |
| A_51_P157986 | NM_029930 | 77574 | 3321401G04Rik | 0.909 | 1.196 | 1.088 | 0.563 | 0.621 | 0.598 |
| A_52_P1013441 | | | | 1.255 | 1.032 | 2.041 | 1.173 | 1.203 | 1.341 |
| A_51_P418375 | NM_023844 | 67374 | Jam2 | 1.044 | 0.943 | 0.99 | 0.826 | 0.786 | 0.84 |
| A_52_P557940 | AK004302; | 66704 | 4921506I22Rik | 0.544 | 0.595 | 0.569 | 1.371 | 1.379 | 1.241 |
| A_51_P480311 | NM_010168 | 14061 | F2 | 0.348 | 0.476 | 0.622 | 0.996 | 1.004 | 1.186 |
| A_52_P200458 | NM_010242 | 14345 | Fut4 | 1.003 | 0.997 | 1.017 | 1.108 | 1.203 | 1.09 |
| A_51_P463846 | NM_145545 | 229900 | 9830147J24Rik | 2.28 | 2.199 | 2.49 | 2.068 | 1.958 | 1.72 |
| A_51_P162474 | AF285577; | 107815 | Scml2 | 2.885 | 2.549 | 2.371 | 3.021 | 3.116 | 3.1 |
| A_51_P165244 | NM_018734 | 55932 | Gbp4 | 2.388 | 1.319 | 2.543 | 2.404 | 2.051 | 2.534 |
| A_52_P59318 | NM_133832 | 98711 | Rdh10 | 1.689 | 1.611 | 1.584 | 0.876 | 1.002 | 0.955 |
| A_52_P438036 | NM_175271 | 78134 | Gpr23 | 1.082 | 0.96 | 0.968 | 1.634 | 1.593 | 1.618 |
| A_52_P474242 | BE457768; | | | 1.31 | 1.785 | 1.55 | 1.137 | 1.132 | 0.997 |
| A_51_P499854 | NM_010284 | 14600 | Ghr | 1.55 | 1.19 | 1.486 | 2.502 | 2.537 | 2.196 |
| A_51_P455647 | NM_009801 | 12349 | Car2 | 1.592 | 1.463 | 1.337 | 2.139 | 2.347 | 2.17 |
| A_52_P103929 | NM_009306 | 20979 | Syt1 | 0.898 | 0.938 | 0.967 | 1.626 | 1.464 | 1.422 |
| A_51_P149469 | NM_009608 | 11464 | Actc1 | 1.012 | 1.006 | 0.935 | 0.971 | 0.997 | 1.12 |
| A_52_P557293 | NM_015775 | 50528 | Tmprss2 | 1.142 | 1.091 | 1.111 | 1.208 | 1.127 | 1.225 |
| A_52_P50496 | AY048587; XM_619453 | 114640 | Tifp39 | 1.196 | 1.551 | 1.504 | 1.114 | 1.171 | 1.214 |
| A_51_P180413 | NM_176930 | 319504 | C130076O07Rik | 1.603 | 1.54 | 1.395 | 1.297 | 1.25 | 1.155 |
| A_51_P229613 | NM_008792 | 18549 | Pcsk2 | 1.463 | 1.53 | 1.37 | 1.333 | 1.422 | 1.554 |
| A_51_P121635 | NM_030700 | 80884 | Maged2 | 1.225 | 1.426 | 1.335 | 0.918 | 0.99 | 0.88 |
| A_51_P481777 | NM_018756 | 54382 | Tcstv1 | 0.208 | 0.696 | 0.654 | 0.592 | 0.503 | 0.72 |
| A_51_P412817 | NM_177617 | 216851 | D330014H01Rik | 0.725 | 0.834 | 1.04 | 1.083 | 0.986 | 0.997 |
| A_51_P387528 | AK019664; | 78801 | Ak7 | 0.65 | 0.537 | 0.813 | 0.767 | 0.778 | 1.11 |
| A_52_P78168 | AK031518; | | | 0.277 | 0.385 | 0.299 | 0.704 | 0.652 | 0.544 |
| A_52_P274960 | NM_001005508 | 226652 | 6030405P05Rik | 0.671 | 0.634 | 0.712 | 0.763 | 0.781 | 0.808 |
| A_52_P1188270 | AK042362; | | | 0.341 | 0.594 | 0.564 | 1.758 | 1.462 | 1.658 |
| A_51_P291227 | NM_001005508 | 226652 | 6030405P05Rik | 0.705 | 0.681 | 0.731 | 0.814 | 0.735 | 0.798 |
| A_52_P377299 | AK048360; | 271377 | Zbtb11 | 0.577 | 0.582 | 0.645 | 1.152 | 1.092 | 1.023 |
| A_51_P297131 | NM_009502 | 22330 | Vcl | 1.015 | 1.436 | 0.955 | 0.556 | 0.672 | 0.587 |
| A_52_P804224 | | | | 0.299 | 0.314 | 0.291 | 1.263 | 1.2 | 1.068 |
| A_51_P442894 | AK048310; | 621603 | C130048D07Rik | 1.023 | 0.841 | 1.218 | 0.788 | 0.867 | 0.977 |
| A_52_P309718 | AK173099; | 218850 | D14Abb1e | 1.006 | 0.813 | 1.127 | 1.186 | 1.29 | 1.289 |
| A_52_P359010 | AK129478; | 72193 | Sfrs2ip | 0.54 | 0.706 | 0.574 | 1.484 | 1.398 | 1.403 |
| A_51_P410813 | AK079732; | | | 0.717 | 0.877 | 0.904 | 1.593 | 1.473 | 1.607 |
| A_52_P382565 | XM_483943 | 432493 | LOC432493 | 0.228 | 0.273 | 0.254 | 0.607 | 0.622 | 0.462 |
| A_51_P333253 | NM_178440 | 246177 | Myo1g | 0.535 | 0.491 | 0.538 | 0.474 | 0.481 | 0.37 |
| A_52_P113190 | NM_053214 | 17916 | Myo1f | 0.386 | 0.513 | 0.397 | 0.243 | 0.232 | 0.264 |
| A_51_P473953 | XM_358600; XM_355454 | 381498 | 4930592C13Rik | 2.138 | 1.593 | 1.947 | 2.323 | 2.226 | 2.26 |

TABLE 6A
25 Gene Subset (Germline Competent)

| | | | | Clone 1 | | | Clone 2 | | |
|---|---|---|---|---|---|---|---|---|---|
| Agilent Probe ID | Genbank | Entrez ID | Gene Symbol | rep. 1 | rep. 2 | rep. 3 | rep. 1 | rep. 2 | rep. 3 |
| A_51_P237040 | NM_008711 | 18121 | Nog | 0.266 | 0.299 | 0.281 | 0.396 | 0.368 | 0.407 |
| A_51_P335460 | NM_009132 | 20259 | Scin | 0.718 | 0.631 | 0.719 | 0.678 | 0.674 | 0.666 |
| A_52_P391505 | NM_144513 | 17263 | Gtl2 | 1.36 | 1.291 | 1.327 | 1.053 | 1.04 | 0.852 |
| A_51_P497100 | NM_010706 | 16855 | Lgals4 | 2.115 | 2.049 | 2.368 | 1.716 | 1.861 | 1.527 |
| A_51_P378371 | | | | 0.454 | 0.542 | 0.39 | 0.468 | 0.484 | 0.468 |
| A_51_P298266 | NM_025681 | 66643 | Lix1 | 0.507 | 0.414 | 0.416 | 0.554 | 0.584 | 0.54 |
| A_52_P269158 | NM_001003948 | 98496 | AL024069 | 0.316 | 0.33 | 0.33 | 0.388 | 0.393 | 0.443 |
| A_51_P311038 | BC009666; XM_134720 | 57267 | Apba3 | 0.334 | 0.341 | 0.339 | 0.446 | 0.472 | 0.482 |
| A_51_P457187 | NM_028075 | 72049 | Tnfrsf13c | 2.82 | 3.275 | 3.026 | 2.654 | 3.432 | 2.546 |
| A_52_P68702 | NM_145148 | 232288 | Frmd4b | 0.545 | 0.476 | 0.538 | 0.501 | 0.43 | 0.424 |
| A_52_P293443 | AK087208; | 13819 | | 1.278 | 1.075 | 1.13 | 1.496 | 1.604 | 1.349 |
| A_51_P157986 | NM_029930 | 77574 | 3321401G04Rik | 0.426 | 0.339 | 0.345 | 0.368 | 0.318 | 0.37 |
| A_52_P1013441 | | | | 3.248 | 3.164 | 3.191 | 4.558 | 5.61 | 4.683 |
| A_51_P418375 | NM_023844 | 67374 | Jam2 | 1.691 | 1.467 | 1.625 | 2.142 | 2.132 | 2.288 |
| A_52_P557940 | AK004302; | 66704 | 4921506I22Rik | 2.114 | 1.863 | 2.221 | 2.17 | 2.013 | 2.051 |
| A_51_P480311 | NM_010168 | 14061 | F2 | 1.991 | 1.809 | 1.969 | 2.465 | 2.4 | 1.874 |
| A_52_P200458 | NM_010242 | 14345 | Fut4 | 0.429 | 0.343 | 0.355 | 0.453 | 0.391 | 0.512 |
| A_51_P463846 | NM_145545 | 229900 | 9830147J24Rik | 0.694 | 0.519 | 0.59 | 0.86 | 0.816 | 0.817 |
| A_51_P162474 | AF285577; | 107815 | Scml2 | 0.539 | 0.413 | 0.405 | 0.643 | 0.645 | 0.71 |
| A_51_P165244 | NM_018734 | 55932 | Gbp4 | 0.585 | 0.512 | 0.474 | 1.008 | 0.981 | 1.006 |
| A_52_P59318 | NM_133832 | 98711 | Rdh10 | 0.306 | 0.273 | 0.275 | 0.373 | 0.395 | 0.445 |
| A_52_P438036 | NM_175271 | 78134 | Gpr23 | 0.376 | 0.334 | 0.36 | 0.455 | 0.441 | 0.489 |
| A_52_P474242 | BE457768; | | | 0.499 | 0.53 | 0.46 | 0.69 | 0.658 | 0.726 |
| A_51_P499854 | NM_010284 | 14600 | Ghr | 0.407 | 0.375 | 0.412 | 0.413 | 0.429 | 0.489 |
| A_51_P455647 | NM_009801 | 12349 | Car2 | 0.335 | 0.288 | 0.301 | 0.568 | 0.516 | 0.638 |

TABLE 6B
25 Gene Subset (Non Germline Competent)

| | | | | Clone 1 | | | Clone 2 | | |
|---|---|---|---|---|---|---|---|---|---|
| Agilent Probe ID | Genbank | Entrez ID | Gene Symbol | rep. 1 | rep. 2 | rep. 3 | rep. 1 | rep. 2 | rep. 3 |
| A_51_P237040 | NM_008711 | 18121 | Nog | 1.152 | 1.407 | 1.101 | 0.859 | 0.96 | 0.924 |
| A_51_P335460 | NM_009132 | 20259 | Scin | 1.658 | 1.913 | 1.519 | 1.101 | 1.225 | 1.107 |
| A_52_P391505 | NM_144513 | 17263 | Gtl2 | 0.0793 | 0.114 | 0.0867 | 0.158 | 0.15 | 0.148 |
| A_51_P497100 | NM_010706 | 16855 | Lgals4 | 0.692 | 0.771 | 0.849 | 1.002 | 0.908 | 0.828 |
| A_51_P378371 | | | | 0.986 | 1.006 | 1.208 | 1.217 | 1.156 | 1.234 |
| A_51_P298266 | NM_025681 | 66643 | Lix1 | 1.123 | 1.145 | 1.109 | 1.143 | 1.15 | 1.141 |
| A_52_P269158 | NM_001003948 | 98496 | AL024069 | 1.775 | 1.419 | 1.506 | 1.494 | 1.391 | 1.254 |
| A_51_P311038 | BC009666; XM_134720 | 57267 | Apba3 | 1.847 | 1.642 | 1.538 | 2.55 | 2.405 | 2.808 |
| A_51_P457187 | NM_028075 | 72049 | Tnfrsf13c | 1.083 | 1.58 | 1.373 | 0.916 | 1.099 | 1.091 |
| A_52_P68702 | NM_145148 | 232288 | Frmd4b | 0.928 | 1.071 | 0.877 | 1.242 | 1.234 | 1.139 |
| A_52_P293443 | AK087208; | 13819 | | 0.179 | 0.222 | 0.219 | 0.543 | 0.565 | 0.552 |
| A_51_P157986 | NM_029930 | 77574 | 3321401G04Rik | 0.909 | 1.196 | 1.088 | 0.563 | 0.621 | 0.598 |
| A_52_P1013441 | | | | 1.255 | 1.032 | 2.041 | 1.173 | 1.203 | 1.341 |
| A_51_P418375 | NM_023844 | 67374 | Jam2 | 1.044 | 0.943 | 0.99 | 0.826 | 0.786 | 0.84 |
| A_52_P557940 | AK004302; | 66704 | 4921506I22Rik | 0.544 | 0.595 | 0.569 | 1.371 | 1.379 | 1.241 |
| A_51_P480311 | NM_010168 | 14061 | F2 | 0.348 | 0.476 | 0.622 | 0.996 | 1.004 | 1.186 |
| A_52_P200458 | NM_010242 | 14345 | Fut4 | 1.003 | 0.997 | 1.017 | 1.108 | 1.203 | 1.09 |
| A_51_P463846 | NM_145545 | 229900 | 9830147J24Rik | 2.28 | 2.199 | 2.49 | 2.068 | 1.958 | 1.72 |
| A_51_P162474 | AF285577; | 107815 | Scml2 | 2.885 | 2.549 | 2.371 | 3.021 | 3.116 | 3.1 |
| A_51_P165244 | NM_018734 | 55932 | Gbp4 | 2.388 | 1.319 | 2.543 | 2.404 | 2.051 | 2.534 |
| A_52_P59318 | NM_133832 | 98711 | Rdh10 | 1.689 | 1.611 | 1.584 | 0.876 | 1.002 | 0.955 |
| A_52_P438036 | NM_175271 | 78134 | Gpr23 | 1.082 | 0.96 | 0.968 | 1.634 | 1.593 | 1.618 |
| A_52_P474242 | BE457768; | | | 1.31 | 1.785 | 1.55 | 1.137 | 1.132 | 0.997 |
| A_51_P499854 | NM_010284 | 14600 | Ghr | 1.55 | 1.19 | 1.486 | 2.502 | 2.537 | 2.196 |
| A_51_P455647 | NM_009801 | 12349 | Car2 | 1.592 | 1.463 | 1.337 | 2.139 | 2.347 | 2.17 |

TABLE 7A
10 Gene Subset (Germline Competent)

| | | | | Clone 1 | | | Clone 2 | | |
|---|---|---|---|---|---|---|---|---|---|
| Agilent Probe ID | Genbank | Entrez ID | Gene Symbol | rep. 1 | rep. 2 | rep. 3 | rep. 1 | rep. 2 | rep. 3 |
| A_51_P237040 | NM_008711 | 18121 | Nog | 0.266 | 0.299 | 0.281 | 0.396 | 0.368 | 0.407 |
| A_51_P335460 | NM_009132 | 20259 | Scin | 0.718 | 0.631 | 0.719 | 0.678 | 0.674 | 0.666 |
| A_52_P391505 | NM_144513 | 17263 | Gtl2 | 1.36 | 1.291 | 1.327 | 1.053 | 1.04 | 0.852 |
| A_51_P497100 | NM_010706 | 16855 | Lgals4 | 2.115 | 2.049 | 2.368 | 1.716 | 1.861 | 1.527 |
| A_51_P378371 | | | | 0.454 | 0.542 | 0.39 | 0.468 | 0.484 | 0.468 |
| A_51_P298266 | NM_025681 | 66643 | Lix1 | 0.507 | 0.414 | 0.416 | 0.554 | 0.584 | 0.54 |
| A_52_P269158 | NM_001003948 | 98496 | AL024069 | 0.316 | 0.33 | 0.33 | 0.388 | 0.393 | 0.443 |
| A_51_P311038 | BC009666; XM_134720 | 57267 | Apba3 | 0.334 | 0.341 | 0.339 | 0.446 | 0.472 | 0.482 |
| A_51_P457187 | NM_028075 | 72049 | Tnfrsf13c | 2.82 | 3.275 | 3.026 | 2.654 | 3.432 | 2.546 |
| A_52_P68702 | NM_145148 | 232288 | Frmd4b | 0.545 | 0.476 | 0.538 | 0.501 | 0.43 | 0.424 |

TABLE 7B
10 Gene Subset (Non Germline Competent)

| | | | | Clone 1 | | | Clone 2 | | |
|---|---|---|---|---|---|---|---|---|---|
| Agilent Probe ID | Genbank | Entrez ID | Gene Symbol | rep. 1 | rep. 2 | rep. 3 | rep. 1 | rep. 2 | rep. 3 |
| A_51_P237040 | NM_008711 | 18121 | Nog | 1.152 | 1.407 | 1.101 | 0.859 | 0.96 | 0.924 |
| A_51_P335460 | NM_009132 | 20259 | Scin | 1.658 | 1.913 | 1.519 | 1.101 | 1.225 | 1.107 |
| A_52_P391505 | NM_144513 | 17263 | Gtl2 | 0.0793 | 0.114 | 0.0867 | 0.158 | 0.15 | 0.148 |
| A_51_P497100 | NM_010706 | 16855 | Lgals4 | 0.692 | 0.771 | 0.849 | 1.002 | 0.908 | 0.828 |
| A_51_P378371 | | | | 0.986 | 1.006 | 1.208 | 1.217 | 1.156 | 1.234 |
| A_51_P298266 | NM_025681 | 66643 | Lix1 | 1.123 | 1.145 | 1.109 | 1.143 | 1.15 | 1.141 |
| A_52_P269158 | NM_001003948 | 98496 | AL024069 | 1.775 | 1.419 | 1.506 | 1.494 | 1.391 | 1.254 |
| A_51_P311038 | BC009666; XM_134720 | 57267 | Apba3 | 1.847 | 1.642 | 1.538 | 2.55 | 2.405 | 2.808 |
| A_51_P457187 | NM_028075 | 72049 | Tnfrsf13c | 1.083 | 1.58 | 1.373 | 0.916 | 1.099 | 1.091 |
| A_52_P68702 | NM_145148 | 232288 | Frmd4b | 0.928 | 1.071 | 0.877 | 1.242 | 1.234 | 1.139 |

TABLE 8A
5 Gene Subset (Germline Competent)

| | | | | Clone 1 | | | Clone 2 | | |
|---|---|---|---|---|---|---|---|---|---|
| Agilent Probe ID | Genbank | Entrez ID | Gene Symbol | rep. 1 | rep. 2 | rep. 3 | rep. 1 | rep. 2 | rep. 3 |
| A_51_P237040 | NM_008711 | 18121 | Nog | 0.266 | 0.299 | 0.281 | 0.396 | 0.368 | 0.407 |
| A_51_P335460 | NM_009132 | 20259 | Scin | 0.718 | 0.631 | 0.719 | 0.678 | 0.674 | 0.666 |
| A_52_P391505 | NM_144513 | 17263 | Gtl2 | 1.36 | 1.291 | 1.327 | 1.053 | 1.04 | 0.852 |
| A_51_P497100 | NM_010706 | 16855 | Lgals4 | 2.115 | 2.049 | 2.368 | 1.716 | 1.861 | 1.527 |
| A_51_P378371 | | | | 0.454 | 0.542 | 0.39 | 0.468 | 0.484 | 0.468 |

TABLE 8B
5 Gene Subset (Non Germline Competent)

| | | | | Clone 1 | | | Clone 2 | | |
|---|---|---|---|---|---|---|---|---|---|
| Agilent Probe ID | Genbank | Entrez ID | Gene Symbol | rep. 1 | rep. 2 | rep. 3 | rep. 1 | rep. 2 | rep. 3 |
| A_51_P237040 | NM_008711 | 18121 | Nog | 1.152 | 1.407 | 1.101 | 0.859 | 0.96 | 0.924 |
| A_51_P335460 | NM_009132 | 20259 | Scin | 1.658 | 1.913 | 1.519 | 1.101 | 1.225 | 1.107 |
| A_52_P391505 | NM_144513 | 17263 | Gtl2 | 0.0793 | 0.114 | 0.0867 | 0.158 | 0.15 | 0.148 |
| A_51_P497100 | NM_010706 | 16855 | Lgals4 | 0.692 | 0.771 | 0.849 | 1.002 | 0.908 | 0.828 |
| A_51_P378371 | | | | 0.986 | 1.006 | 1.208 | 1.217 | 1.156 | 1.234 |

Although the foregoing invention has been described in some detail by way of illustration and examples, it will be readily apparent to those of ordinary skill in the art that certain changes and modifications may be made to the teachings of the invention without departing from the spirit or scope of the appended claims.

1. A method for selecting a genetically modified ES cell that is germline competent, comprising providing a first expression profile of a subset of ES cell genes from a genetically modified ES cell and comparing the first expression profile of the subset of ES cell genes from the genetically modified ES cell with a second expression profile of a subset of ES cell genes from a germline competent ES cell, wherein the first subset of ES cell genes and the second subset of ES cell genes are selected from the subset of genes listed in Table 8 in conjunction with one or more different genes selected from the genes listed in Table 5.

2. The method of claim 1, wherein the first and the second expression profile is generated on an expression array.

3. The method of claim 1, wherein the first and the second expression profile is generated by an ELISA method or a protein expression assay.

4. The method of claim 1, wherein the first and the second expression profile is for a single gene.

5. The method of claim 1, wherein the first and the second expression profile is a profile of expressed and secreted genes.

6. The method of claim 1, wherein the first and the second expression profile is generated using an ELISA assay.

7. The method of claim 1, wherein the first and the second expression profile is generated using an affinity assay using microspheres in the liquid phase.

8. The method according to claim 1, wherein the subset of genes is selected from the group consisting of 50 genes, 25 genes, 10 genes, 5 genes, and 2 genes.

9. A method for selecting a genetically modified ES cell that is germline competent, comprising ascertaining the level of expression of a single gene of the genetically modified ES cell, wherein the level of expression of the single gene differs at least two-fold between a germline competent ES cell and a non-germline competent ES cell, and the single gene is selected from the genes listed in Table 5.

10. The method of claim 9, wherein the expression is up-regulated at least two-fold in the germline competent ES cell.

11. The method of claim 9, wherein the expression is down-regulated at least two-fold in the germline competent ES cell.

12. The method of claim 9, wherein the single gene is an imprinted, maternally expressed gene which is expresses untranslated mRNA.

13. The method of claim 12, wherein the single gene is gtl2.
